(19) United States
(12) Patent Application Publication
(10) Pub. No.: US 2021/0317535 A1
(43) Pub. Date: Oct. 14, 2021

(54) METHODS OF DETERMINING TREATMENT CONSISTING OF RADIATION THERAPY AND/OR ALKYLATING CHEMOTHERAPY IN PATIENTS SUFFERING FROM CANCER

(71) Applicants: THE REGENTS OF THE UNIVERSITY OF MICHIGAN, Ann Arbor, MI (US); MAYO FOUNDATION FOR MEDICAL EDUCATION AND RESEARCH, Rochester, MN (US)

(72) Inventors: Daniel Wahl, Ann Arbor, MI (US); Shuang Zhao, Ann Arbor, MI (US); Jann Sarkaria, Rochester, MN (US)

(21) Appl. No.: 17/271,981

(22) PCT Filed: Aug. 29, 2019
(86) PCT No.: PCT/US2019/048818

§ 371 Date: Feb. 26, 2021

Related U.S. Application Data
(60) Provisional application No. 62/724,337, filed on Aug. 29, 2018.

(51) Int. Cl. C12Q 1/6886; A61P 35/00; A61K 31/495; A61K 31/17; G16B 40/00; A61N 5/10

(52) U.S. Cl. CPC C12Q 1/6886; A61P 35/00; A61K 31/495; A61K 31/17; C12Q 2600/158; A61N 5/10; C12Q 2600/112; C12Q 2600/118; G16B 40/00

(57) ABSTRACT

Methods of treating a tumor in a subject are provided herein. In exemplary embodiments, the method comprises measuring the expression level of MGMT, GPRASP1, CHGA, MAPK8, ATP6V0A2, or FGF7, or any combination thereof, in a sample comprising a cell or cells from the tumor, and administering to the subject an alkylating chemotherapy, radiation therapy, or a combination comprising alkylating chemotherapy and radiation therapy, depending on the expression level(s). Related methods, kits, systems, computer-readable storage media, and methods implemented by a processor in a computer are further provided herein.

### CROSS-REFERENCE TO RELATED APPLICATIONS

This application claims priority to U.S. Provisional Patent Application No. 62/724,337, filed Aug. 29, 2018, the entire contents of which is incorporated by reference.

### FEDERAL GRANT STATEMENT

This invention was made with government support under CA108961 awarded by the National Institutes of Health. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 180,933 byte ASCII (Text) file named “52817A_Seqlisting.txt”; created on Aug. 29, 2019.

### BACKGROUND

Genomic biomarkers are promising tools to personalize therapy for patients with cancer¹. Prognostic biomarkers provide insight into disease natural history but do not necessarily predict the benefit derived by a particular therapy. Predictive biomarkers provide insight into the benefit a patient might receive from a specific therapy and therefore are useful to medical professionals during selection of an appropriate treatment for a given patient. Unlike prognostic biomarkers, which are manifold, predictive biomarkers are few in number—only a few prospectively-validated predictive biomarkers exist in oncology and none exist for radiotherapy (RT) or chemotherapy and radiotherapy (ChemoRT).

Following surgery, most patients with glioblastoma (GBM) are treated with a combination of temozolomide (TMZ) and RT, while some receive only one of these treatments. Molecular information to guide the selection of these treatment options is lacking. Among the commonly performed molecular analyses in GBM, MGMT promoter methylation may predict for treatment benefit from TMZ. However, this analysis fails to provide information regarding response to RT, or multi-modality therapy versus single-modality or no treatment³⁻⁶. Mutations of isocitrate dehydrogenase 1 (IDH1) are prognostic in GBM. However, such biomarkers do not predict for response to standard treatments⁷. Thus, a set of biomarkers that can predict expected benefit from all treatment modalities is needed.

### SUMMARY

Presented here for the first time are data demonstrating the validity of gene signatures which predict treatment response in a patient. In a study first of its kind, RNAseq was performed on a large cohort of GBM patient-derived xenografts (PDXs) at baseline. These PDXs were treated with RT, TMZ or the combination of RT and TMZ (RT+TMZ) and gene signatures (GS) predicting treatment response (termed RT-GS, Chemo-GS, and ChemoRT-GS) were developed. The gene signatures were then independently validated in The Cancer Genome Atlas (TCGA) GBM cohort to assess the clinical performance of the GS as predictive biomarkers, and compared our results to MGMT promoter methylation and gene expression.

Accordingly, the present disclosure provides methods of treating a tumor in a subject. In exemplary embodiments, the subject is one from whom a sample comprising a cell or cells from the tumor was obtained, and the expression level of MGMT, GPRASP1, CHGA, MAPK8, ATP6V0A2, FGF7, or any combination thereof, of the sample was measured, and the method comprises administering to the subject (i) an alkylating chemotherapy, (ii) radiation therapy, or (iii) a combination of alkylating chemotherapy and radiation therapy in an amount effective to treat the tumor, based on the expression level.

In exemplary embodiments of the presently disclosed method of treating a tumor, the subject has a decreased expression level of MGMT or GPRASP1, or both, relative to a reference level. In exemplary instances, the method comprises administering to the subject an alkylating chemotherapy in an amount effective to treat the tumor.

In exemplary embodiments of the presently disclosed method of treating a tumor, the subject has an increased expression level of CHGA or MAPK8, or both, relative to a reference level. In exemplary instances, the method comprises administering to the subject a radiation therapy in an amount effective to treat the tumor.

In exemplary embodiments of the presently disclosed method of treating a tumor, the subject has (A) a decreased expression level of MGMT, GPRASP1, ATP6V0A2, FGF7, or any combination thereof, relative to a reference level, or (B) an increased expression level of CHGA or MAPK8, or both, relative to a reference level, or (C) both (A) and (B). In exemplary aspects, the method comprises administering to the subject a combination comprising an alkylating chemotherapy and a radiation therapy in an amount effective to treat the tumor.

In exemplary embodiments of the presently disclosed method of treating a tumor, the method comprises (a) measuring the expression level of MGMT, GPRASP1, CHGA, MAPK8, ATP6V0A2, FGF7, or any combination thereof, in a sample comprising a cell or cells from the tumor, and (b) administering to the subject: (i) an alkylating chemotherapy, (ii) radiation therapy, or (iii) a combination comprising an alkylating chemotherapy and a radiation therapy, depending on the expression level(s) measured in (a). In some aspects, an alkylating chemotherapy is administered when the expression level of MGMT, GPRASP1, or both, in the sample is decreased relative to a reference level. In other aspects, a radiation therapy is administered, when the expression level or CHGA, MAPK8, or both, in the sample is increased relative to a reference level. In yet other aspects, a combination comprising an alkylating chemotherapy and a radiation therapy is administered, when (A) the expression level of MGMT, GPRASP1, ATP6V0A2, or FGF7, or any combination thereof, in the sample is decreased, relative to a reference level or (B) the expression level of CHGA, MAPK8, or both, in the sample is increased relative to a reference level or (C) a both (A) and (B).

The present disclosure also provides methods of identifying a tumor in a subject as treatable with a particular type of therapy. Without being bound to any particular theory, the methods provided herein allow for a medical professional to predict the expected benefit from a specific treatment. In exemplary embodiments, the treatment comprises an alkylating chemotherapy and the method is a method of identifying a tumor in a subject as treatable with and/or responsive to an alkylating chemotherapy. In exemplary instances, the method comprises (a) measuring the level of expression of MGMT or GPRASP1, or both, in a sample comprising a cell or cells from the tumor, and (b) identifying the tumor as treatable with and/or responsive to an alkylating chemotherapy, when the level of MGMT, GPRASP1, or both, in the sample is decreased relative to a reference level. In exemplary embodiments, the treatment comprises radiation therapy and the method is a method of identifying a tumor in a subject as treatable with and/or responsive to a radiation therapy. In exemplary aspects, the method comprises (a) measuring the level of expression of CHGA or MAPK8, or both, in a sample comprising a cell or cells from the tumor, and (b) identifying the tumor as treatable with and/or responsive to radiation therapy, when the level of CHGA, MAPK8, or both, in the sample is increased relative to a reference level. In exemplary embodiments, the treatment comprises a combination comprising alkylating chemotherapy and radiation therapy and the method is a method of identifying a tumor in a subject as treatable with and/or responsive to a combination comprising alkylating chemotherapy and radiation therapy. In exemplary aspects, the method comprises (a) measuring the level of expression of MGMT, GPRASP1, CHGA, MAPK8, ATP6V0A2, FGF7, or any combination thereof, in a sample comprising a cell or cells from the tumor, and (b) identifying the subject with a tumor treatable with and/or responsive to the combination, when (i) the expression level of MGMT, GPRASP1, ATP6V0A2, or FGF7, or any combination thereof, in the sample is decreased relative to a reference level, (ii) the expression level of CHGA or MAPK8, or both, in the sample is increased relative to a reference level, or (iii) both (i) and (ii).

Further provided are methods of determining treatment for a subject with a tumor. In exemplary embodiments, the subject is one from whom a sample comprising a cell or cells from the tumor was obtained and the expression level of MGMT, GPRASP1, CHGA, MAPK8, ATP6V0A2, FGF7, or any combination thereof, of the sample was measured, and the method comprises selecting for the subject (i) an alkylating chemotherapy, (ii) radiation therapy, or (iii) a combination of alkylating chemotherapy and radiation therapy, depending on the measured expression level(s). In exemplary embodiments, the method comprises (a) measuring the level of expression of MGMT, GPRASP1, CHGA, MAPK8, ATP6V0A2, FGF7, or any combination thereof, in a sample comprising a cell or cells from the tumor, and (b) selecting for the subject a treatment comprising (i) an alkylating chemotherapy, (ii) radiation therapy, or (iii) a combination comprising an alkylating chemotherapy and a radiation therapy, depending on the expression level(s) measured in (a). In some aspects, an alkylating chemotherapy is selected for the subject when the expression level of MGMT, GPRASP1, or both, in the sample is decreased relative to a reference level. In other aspects, a radiation therapy is selected for the subject, when the expression level or CHGA, MAPK8, or both, in the sample is increased relative to a reference level. In yet other aspects, a combination comprising an alkylating chemotherapy and a radiation therapy is selected for the subject, when (A) the expression level of MGMT, GPRASP1, ATP6V0A2, or FGF7, or any combination thereof, in the sample is decreased, relative to a reference level or (B) the expression level of CHGA, MAPK8, or both, in the sample is increased relative to a reference level or (C) a both (A) and (B).

The present disclosure also provides kits comprising at least two nucleic acid probes specific for at least two of MGMT, GPRASP1, CHGA, MAPK8, ATP6V0A2, and FGF7.

Related systems, computer-readable storage media and methods implemented by a processor in a computer are additionally provided.

### BRIEF DESCRIPTION OF THE DRAWINGS

FIG. 1 is a flow diagram detailing the analysis pipeline.

FIGS. 2A-2C depicts an assessment of performance of Chemo-GS in TCGA. Chemotherapy is defined as alkylating chemotherapy with or without RT. No chemotherapy is defined as RT alone or no treatment. (FIG. 2A) Kaplan-meier curves showing that Chemo-GS is not prognostic in patients who did not receive chemotherapy. (FIG. 2B) Kaplan-meier curves showing that Chemo-GS is prognostic in patients who received chemotherapy. (FIG. 2C) Logistic regression interaction plot showing that patients with a higher Chemo-GS score derived more benefit from chemotherapy.

FIGS. 3A-3C depicts an assessment of performance of RT-GS in TCGA. RT is defined as RT with or without chemotherapy. No RT is defined as chemotherapy alone or no treatment. (FIG. 3A) Kaplan-meier curves showing that RT-GS is not prognostic in patients who did not receive RT. (FIG. 3B) Kaplan-meier curves showing that RT-GS is prognostic in patients who received RT. (FIG. 3C) Logistic regression interaction plot showing that patients with a higher RT-GS score derived more benefit from RT.

FIGS. 4A-4C depicts an assessment of performance of ChemoRT-GS in TCGA. ChemoRT is defined as alkylating chemotherapy with RT. No ChemoRT is defined as chemo alone, RT alone, or no treatment. (FIG. 4A) Kaplan-meier curves showing that ChemoRT-GS is not prognostic in patients who did not receive ChemoRT. (FIG. 4B) Kaplan-meier curves showing that ChemoRT-GS is prognostic in patients who received ChemoRT. (FIG. 4C) Logistic regression interaction plot showing that patients with a higher ChemoRT-GS score derived more benefit from ChemoRT.

FIGS. 5A-5B depicts (FIG. 5A) Boxplots showing range of survival ratios between treated and untreated PDXs for RT, TMZ, and RT+TMZ. (FIG. 5B) Kaplan Meier curves showing survival of the PDXs with no treatment, RT, TMZ, and RT+TMZ.

FIG. 6 depicts overlap between the top 100 most positively and negatively correlated genes with treatment response

FIG. 7 is a GSEA performed on gene lists ranked by correlation to treatment response in the PDX. Red=EMT; Orange=ECM; Green=RAS signaling; Blue=DNA replication.

FIG. 8 is a set of Kaplan Meier curves showing survival in TCGA with no treatment, RT, chemotherapy, and ChemoRT

FIG. 9 depicts an assessment of performance of MGMT promoter methylation and expression in TCGA. Chemotherapy is defined as alkylating chemotherapy with or without RT. No chemotherapy is defined as RT alone or no treatment. Kaplan-meier curves showing that both MGMT promoter methylation and expression are not prognostic in patients who did not receive chemotherapy, and borderline prognostic in patients who did receive chemotherapy.

FIG. 10 depicts an assessment of performance of MGMT promoter methylation in TCGA. ChemoRT is defined as alkylating chemotherapy with RT. No ChemoRT is defined as chemo alone, RT alone, or no treatment. Kaplan-meier curves showing that both MGMT promoter methylation and expression are not prognostic in patients who did not receive ChemoRT, and prognostic or borderline prognostic in patients who did receive ChemoRT

FIG. 11 is a system diagram of a processing system for performing the techniques described herein, including, assessing a subject's metastatic potential, in accordance with an example.

FIG. 12 is a table referred to in the Examples as Table S1.

FIGS. 13A-13C are a set of tables referred to in the Examples as Table S2. FIG. 13A is a table listing the top 100 genes correlated with TMZ benefit. FIG. 13B is a table listing the top 100 genes correlated with RT benefit. FIG. 13C is a table listing the top 100 genes correlated with TMZ+RT benefit.

FIG. 14 is a table referred to in the Examples as Table S3.

FIG. 15 is a table referred to in the Examples as Table S4.

FIG. 16 is a table referred to in the Examples as Table S5.

### DETAILED DESCRIPTION

Randomized control trials provide the ideal study design to develop and validate predictive biomarkers, but costs and sample scarcity limit feasibility. In vitro experimentation on cancer cell lines is more feasible but has significant biologic limitations^{8,9}. Orthotopic patient-derived xenografts (PDXs), in which tumor tissue directly from patients is implanted into the relevant body site in mice, represent an improved model system that recapitulates much of the biology of human tumors including the microenvironment, intratumoral heterogeneity, and, in GBM, the blood brain barrier¹⁰. Orthotopic PDXs typically recapitulate the treatment-responsiveness of their founder tumors¹¹ and can be used to assess individual biomarkers¹²⁻¹⁴. However, to our knowledge, there are no reported studies utilizing large numbers of PDXs combined with high-throughput gene expression profiling as a strategy to identify predictive biomarkers for treatment response. Using the gene expression and treatment response data from the PDXs gene signatures for RT, chemotherapy, and ChemoRT response were developed and then independently validated in TCGA.

Methods of Determining a Treatment and Tumor Identification

The present disclosure provides methods of determining treatment for a subject with a tumor. In exemplary embodiments, the subject is one from whom a sample comprising a cell or cells from the tumor was obtained and the expression level of MGMT, GPRASP1, CHGA, MAPK8, ATP6V0A2, FGF7, or any combination thereof, of the sample was measured, and the method comprises selecting for the subject (i) an alkylating chemotherapy, (ii) radiation therapy, or (iii) a combination of alkylating chemotherapy and radiation therapy, depending on the measured expression level(s). In exemplary aspects, the expression levels of (A) MGMT and GPRASP1 or (B) CHGA and MAPK8 were measured, and, optionally, the method comprises selecting for the subject (A) an alkylating chemotherapy, when the measured expression levels of MGMT and GPRASP1 in the sample were decreased relative to a reference level, or (B) radiation therapy, when the measured expression levels of CHGA and MAPK8 in the sample were increased relative to a reference level. In additional or alternative exemplary instances, the expression levels of MGMT, GPRASP1, CHGA, MAPK8, ATP6V0A2, and FGF7 were measured, and, optionally, the method comprises selecting for the subject a combination of alkylating chemotherapy and radiation therapy, when the measured expression levels of MGMT, GPRASP1, ATP6V0A2, and FGF7 in the sample were decreased, relative to a reference level, and/or the measured expression levels of CHGA and MAPK8 in the sample were increased, relative to a reference level.

In exemplary embodiments, the method comprises (a) measuring the level of expression of MGMT, GPRASP1, CHGA, MAPK8, ATP6V0A2, FGF7, or any combination thereof, in a sample comprising a cell or cells from the tumor, and (b) selecting for the subject a treatment comprising (i) an alkylating chemotherapy, (ii) radiation therapy, or (iii) a combination comprising an alkylating chemotherapy and a radiation therapy, depending on the expression level(s) measured in (a). In exemplary aspects, the method comprises measuring the expression levels of (A) MGMT and GPRASP1 or (B) CHGA and MAPK8, and, optionally, the method comprises selecting for the subject a treatment comprising: (1) an alkylating chemotherapy, when the measured expression levels of MGMT and GPRASP1 in the sample are decreased relative to a reference level, or (2) radiation therapy, when the measured expression levels of CHGA and MAPK8 in the sample are increased relative to a reference level. In alternative or additional aspects, the method comprises measuring the expression levels of MGMT, GPRASP1, CHGA, MAPK8, ATP6V0A2, and FGF7, and, optionally, the method comprises selecting for the subject a treatment comprising a combination of alkylating chemotherapy and radiation therapy, when the measured expression levels of MGMT, GPRASP1, ATP6V0A2, and FGF7 in the sample are decreased, relative to a reference level, and/or the measured expression levels of CHGA and MAPK8 in the sample are increased, relative to a reference level

The present disclosure also provides methods of identifying a tumor as treatable with and/or responsive to a particular type of therapy. Without being bound to any particular theory, the methods provided herein allow for a medical professional to predict the expected benefit from a specific treatment. In exemplary embodiments, the treatment comprises an alkylating chemotherapy and the method is a method of identifying a tumor as treatable with and/or responsive to an alkylating chemotherapy. In exemplary instances, the method comprises (a) measuring the level of expression of MGMT or GPRASP1, or both, in a sample comprising a cell or cells from the tumor, and (b) identifying the tumor as treatable with and/or responsive to an alkylating chemotherapy, when the level of MGMT, GPRASP1, or both, in the sample is decreased relative to a reference level. In exemplary embodiments, the treatment comprises radiation therapy and the method is a method of identifying a tumor as treatable with and/or responsive to a radiation therapy. In exemplary aspects, the method comprises (a) measuring the level of expression of CHGA or MAPK8, or both, in a sample comprising a cell or cells from the tumor, and (b) identifying the tumor as treatable with and/or responsive to radiation therapy, when the level of CHGA, MAPK8, or both, in the sample is increased relative to a reference level. In exemplary embodiments, the treatment comprises a combination comprising alkylating chemotherapy and radiation therapy and the method is a method of identifying a tumor as treatable with and/or responsive to a combination comprising alkylating chemotherapy and radiation therapy. In exemplary aspects, the method comprises (a) measuring the level of expression of MGMT, GPRASP1, CHGA, MAPK8, ATP6V0A2, FGF7, or any combination thereof, in a sample comprising a cell or cells from the tumor, and (b) identifying the subject with a tumor treatable with and/or responsive to the combination, when (i) the expression level of MGMT, GPRASP1, ATP6V0A2, or FGF7, or any combination thereof, in the sample is decreased relative to a reference level, (ii) the expression level of CHGA or MAPK8, or both, in the sample is increased relative to a reference level, or (iii) both (i) and (ii).

Genes and Measuring Expression Levels

The methods of the present disclosure relate to measuring a level of expression of a gene, an RNA, e.g., a messenger RNA (mRNA), or a protein, in a sample obtained from a subject. In exemplary aspects of the presently disclosed methods, the method comprises measuring the level of expression of MGMT, GPRASP1, CHGA, MAPK8, ATP6V0A2, or FGF7, or any combination thereof. In exemplary aspects, the method comprises measuring the level of gene expression of MGMT, GPRASP1, CHGA, MAPK8, ATP6V0A2, or FGF7, measuring the level of an RNA of MGMT, GPRASP1, CHGA, MAPK8, ATP6V0A2, or FGF7, or measuring the level of a protein of MGMT, GPRASP1, CHGA, MAPK8, ATP6V0A2, or FGF7, or any combination thereof. In exemplary aspects, the method comprises measuring the level of a cDNA based on the RNA of MGMT, GPRASP1, CHGA, MAPK8, ATP6V0A2, or FGF7. In exemplary aspects of the presently disclosed methods, the method comprises measuring the level of expression of one of MGMT, GPRASP1, CHGA, MAPK8, ATP6V0A2, or FGF7. In exemplary aspects, the method comprises measuring the level of expression of at least two or three of MGMT, GPRASP1, CHGA, MAPK8, ATP6V0A2, and FGF7. For example, in some aspects, the method comprises measuring the level of MGMT and GPRASP1 or CHGA and MAPK8. In exemplary aspects, the method comprises measuring the level of expression of at least four or five of MGMT, GPRASP1, CHGA, MAPK8, ATP6V0A2, and FGF7. In exemplary aspects, the method comprises measuring the level of expression of all six of MGMT, GPRASP1, CHGA, MAPK8, ATP6V0A2, and FGF7. Such genes, as well as the RNA and proteins encoded by the genes, are known in the art. The sequences of each are available at the website for the National Center for Biotechnology Information (see Table A), some sequences of which are provided in the sequence listing submitted herewith.

TABLE A

| Gene name | NCBI | | | | |
|---|---|---|---|---|---|
| (abbreviation, | Gene | | | | |
| full) | ID No. | mRNA Accession | SEQ ID NO: | Protein Accession | SEQ ID NO: |
| MGMT | 4255 | XM_005252682.2 | 1 | XP_005252739.1 | 2 |
| Isoform X1 | | | | | |
| MGMT | 4255 | XM_017016275.1 | 3 | XP_016871764.1 | 4 |
| Isoform X1 | | | | | |
| GPRASP1 | 9737 | NM_014710.4 | 5 | NP_055525.3 | 6 |
| Transcript | | | | | |
| Variant 1 | | | | | |
| GPRASP1 | 9737 | NM_001099410.1 | 7 | NP_001092880.1 | 8 |
| Transcript | | | | | |
| Variant 2 | | | | | |
| GPRASP1 | 9737 | NM_001099411.1 | 9 | NP_001092881.1 | 10 |
| Transcript | | | | | |
| Variant 3 | | | | | |
| GPRASP1 | 9737 | NM_001184727.1 | 11 | NP_001171656.1 | 12 |
| Transcript | | | | | |
| Variant 4 | | | | | |
| CHGA | 1113 | NM_001275.3 | 13 | NP_001266.1 | 14 |
| Transcript | | | | | |
| Variant 1 | | | | | |
| CHGA | 1113 | NM_001301690.1 | 15 | NP_001288619.0 | 16 |
| Transcript | | | | | |
| Variant 2 | | | | | |
| MAPK8 | 5599 | NM_001278547.1 | 17 | NP_001265476.1 | 18 |
| Isoform Beta2 | | | | | |
| MAPK8 | 5599 | NM_001278548.1 | 19 | NP_001265477.1 | 20 |
| Isoform 5 | | | | | |
| MAPK8 | 5599 | NM_001323302.1 | 21 | NP_001310231.1 | 22 |
| Isoform Alpha1 | | | | | |
| MAPK8 | 5599 | NM_001323320.1 | 23 | NP_001310249.1 | 24 |
| Isoform 6 | | | | | |
| MAPK8 | 5599 | NM_001323321.1 | 25 | NP_001310250.1 | 26 |
| Isoform Beta1 | | | | | |
| ATP6V0A2 | 23545 | NM_012463.3 | 27 | NP_036595.2 | 28 |
| FGF7 | 2252 | NM_002009.3 | 29 | NP_002000.1 | 30 |

In exemplary embodiments, the methods comprise measuring additional genes, RNA, and/or proteins not listed in Table A. In exemplary embodiments, the methods comprise measuring the expression level of at least one additional gene, RNA, or protein. In exemplary instances, the methods comprise measuring the expression level of at least 2, 3, 4, 5 or more additional genes, at least 2, 3, 4, 5 or more additional RNA, and/or at least 2, 3, 4, 5 or more additional proteins in the sample. In exemplary instances, the methods comprise measuring the expression level of at least 10, 15, 20 or more additional genes, at least 10, 15, 20 or more additional RNA, and/or at least 10, 15, 20 or more additional proteins in the sample. In exemplary instances, the methods comprise measuring the expression level of at least 50, 100, 200 or more additional genes, at least 50, 100, 200 or more additional RNA, and/or at least 50, 100, 200 or more additional proteins in the sample. In exemplary instances, the methods comprise measuring the expression level of a plurality of different genes, a plurality of RNA, and/or a plurality of proteins, in addition to one or more listed in Table A.

Suitable methods of determining expression levels of nucleic acids (e.g., genes, RNA, mRNA) are known in the art and include but not limited to, quantitative polymerase chain reaction (qPCR) (e.g., quantitative real-time PCR (qRT-PCR)), RNAseq, and Northern blotting. Techniques for measuring gene expression include, for example, gene expression assays with or without the use of gene chips or gene expression microarrays are described in Onken et al., J Molec Diag 12(4): 461-468 (2010); and Kirby et al., Adv Clin Chem 44: 247-292 (2007). Affymetrix gene chips and RNA chips and gene expression assay kits (e.g., Applied Biosystems™ TaqMan® Gene Expression Assays) are also commercially available from companies, such as ThermoFisher Scientific (Waltham, Mass.). Suitable methods of determining expression levels of proteins are known in the art and include immunoassays (e.g., Western blotting, an enzyme-linked immunosorbent assay (ELISA), a radioimmunoassay (RIA), and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLoS ONE 3(7): e2535. Proteomic analysis which is the systematic identification and quantification of proteins of a particular biological system are known. Mass spectrometry is typically the technique used for this purposes. Suitable methods of measuring expression are described herein. See the section entitled EXAMPLES. In exemplary aspects, the methods of the present disclosure comprises extracting or isolating RNA from the sample (e.g., from the tumor cell(s) of the sample) and synthesizing cDNA based on RNA isolated from tumor cells of the sample. Accordingly, in some aspects, measuring the expression level comprises isolating RNA from the sample and quantifying the RNA by RNA-Seq. Alternatively or additionally, in some aspects, measuring the expression level comprises isolating RNA from the sample, producing complementary DNA (cDNA) from the RNA, amplifying the cDNA and hybridizing the cDNA to a gene expression microarray. Such arrays are known in the art, some of which are described herein in the Examples.

In alternative or additional aspects, the level of expression is determined via immunohistochemical assays. In exemplary aspects, measuring the expression level comprises contacting the sample with a combination of binding agents to MGMT, GPRASP1, CHGA, MAPK8, ATP6V0A2, FGF7, or a combination thereof. In some aspects, the binding agent is an antibody, or antigen-binding fragment thereof. In some aspects, the binding agent is a nucleic acid probe specific for MGMT, GPRASP1, CHGA, MAPK8, ATP6V0A2, or FGF7, or a complement thereof.

Once the expression level of MGMT, GPRASP1, CHGA, MAPK8, ATP6V0A2, or FGF7 is measured from the sample obtained from the subject, the measured expression level may be compared to a reference level, normalized to a housekeeping gene, mathematically transformed. In exemplary instances, the measured expression level of MGMT, GPRASP1, CHGA, MAPK8, ATP6V0A2, or FGF7 is centered and scaled. Suitable techniques of centering and scaling biological data are known in the art. See, e.g., van den Berg et al., BMC Genomics 7: 142 (2006). In exemplary aspects, the expression of one or more of MGMT, GPRASP1, CHGA, MAPK8, ATP6V0A2, and FGF7 are measured and each level is centered and scaled relative to a reference level. In some, aspects, the expression level of at least two or three (if not, four or five) of MGMT, GPRASP1, CHGA, MAPK8, ATP6V0A2, and FGF7 are measured and each level is centered and scaled relative to a reference level. In some instances, the expression level of each of MGMT, GPRASP1, CHGA, MAPK8, ATP6V0A2, and FGF7 is measured and each measured expression level is centered and scaled relative to a reference level. In exemplary aspects, the reference level is the corresponding expression level of MGMT, GPRASP1, CHGA, MAPK8, ATP6V0A2, or FGF7 in a population untreated subjects. For instance, the measured expression level of MGMT is centered and scaled relative to a reference level, e.g., the expression level of MGMT of a population of untreated subjects with tumors. In exemplary aspects, the measured expression level of GPRASP1 is centered and scaled relative to a reference level, e.g., the expression level of GPRASP1 of a population of untreated subjects with tumors. In some aspects, the measured expression level of CHGA is centered and scaled relative to a reference level, e.g., the expression level of CHGA of a population of untreated subjects with tumors, or the measured expression level of MAPK8 is centered and scaled relative to a reference level, e.g., the expression level of MAPK8 of a population of untreated subjects with tumors, or the measured expression level of ATP6V0A2 is centered and scaled relative to a reference level, e.g., the expression level of ATP6V0A2 of a population of untreated subjects with tumors, or the measured expression level of FGF7 is centered and scaled relative to a reference level, e.g., the expression level of FGF7 of a population of untreated subjects with tumors. In exemplary instances, the centering and scaling of each measured expression level comprises (A) determining the mean of the corresponding expression level of MGMT, GPRASP1, CHGA, MAPK8, ATP6V0A2, or FGF7 of a population of subjects, (B) calculating the standard deviation of the corresponding expression level of MGMT, GPRASP1, CHGA, MAPK8, ATP6V0A2, or FGF7 of the population; (C) subtracting the mean determined in step (A) from the measured expression level to obtain a mean-adjusted expression level, and (D) dividing the mean-adjusted expression level calculated in step (C) by the standard deviation calculated in (B) to obtain a centered and scaled expression level.

In exemplary aspects, the expression levels, e.g., the centered and scaled expression levels, are further processed through a scoring system to obtain a single metric or single score of gene expression, RNA expression, or protein expression. In exemplary aspects, the method comprises calculating:

(A) an alkylating chemotherapy score using Equation 1,

$$\frac{( - 1 ) ( GPRASP 1 ) + ( - 1 ) ( MGMT )}{2} \qquad ( Equation 1 )$$

(B) a radiation therapy score using Equation 2:

$$\frac{( CHGA ) + ( MAPK 8 )}{2} ; \qquad ( Equation 2 )$$

and/or

(C) a combination alkylating chemotherapy/radiation therapy score by using Equation 3:

$$\frac{\begin{matrix}( - 1 ) ( GPRASP 1 ) + ( - 1 ) ( MGMT ) + C H GA + \\ MAPK 8 + ( - 1 ) ( ATP 6 V 0 A 2 ) + ( - 1 ) ( FGF 7 )\end{matrix}}{6} \qquad ( Equation 3 )$$

wherein the centered and scaled expression level for MGMT, GPRASP1, CHGA, MAPK8, ATP6V0A2, and/or FGF7 is used in the above equations.
In exemplary aspects, an alkylating chemotherapy score, a radiation therapy score, and a combination alkylating chemotherapy/radiation therapy score is calculated. In some instances, the method further comprises using each of the above scores to determine a percent chance of overall survival upon treatment with alkylating chemotherapy, a percent chance of overall survival upon treatment with radiation therapy, and/or a percent chance of overall survival upon treatment with a combination of alkylating chemotherapy and radiation therapy. In some aspects, the percent chance of survival with a particular treatment is determined by referencing a table, graph, or database comprising data that associate the predicted or expected percent chance of survival upon a given treatment with a score. For instance, in some instances, the percent chance of survival upon treatment with an alkylating chemotherapy is determined by referencing a table, graph, or database comprising data that associate the percent chance of survival upon alkylating chemotherapy with an alkylating chemotherapy score, or the percent chance of survival upon treatment with a radiation therapy is determined by referencing a table, graph, or database comprising data that associate the percent chance of survival upon radiation therapy with a radiation therapy score, or the percent chance of survival upon treatment with a combination of alkylating chemotherapy and radiation therapy is determined by referencing a table, graph, or database comprising data that associate the percent chance of survival upon the combination therapy with a combination alkylating chemotherapy/radiation therapy score. See, the Examples for a further description of how such graphs may be created. The data may be data of populations of subjects with a known outcome, e.g., subjects having a tumor treatable with and/or responsive to a particular treatment, e.g., alkylating chemotherapy, radiation therapy, combination therapy. In some instances, the method further comprises comparing the percent chance of overall survival upon treatment with alkylating chemotherapy, the percent chance of overall survival upon treatment with radiation therapy and the percent chance of overall survival upon treatment with a combination of alkylating chemotherapy and radiation therapy, and selecting treatment for the subject with the tumor based on the highest percent chance. Example 4 describes an exemplary method of obtaining scores and determining treatment based on the scores. In exemplary aspects, an alkylating chemotherapy score, a radiation therapy score, and a combination alkylating chemotherapy/radiation therapy score are calculated and the score associated with the highest % chance of survival upon a particular treatment is the treatment determined for that subject. In alternative or additional exemplary aspects, the % chance of survival upon a particular treatment is compared to the % chance of survival without the treatment. In exemplary aspects, the decision to have the subject take a particular treatment vs. not take the particular treatment depends on the difference between the % chance of survival upon taking the treatment and the % chance of survival not taking the treatment. If the difference is not substantially large, then a physician and/or the subject may decide not to take the treatment. In exemplary aspects, the method further comprises comparing (A) the difference in the percent chance of overall survival for treatment with alkylating chemotherapy to percent chance of overall survival for treatment without alkylating chemotherapy to (B) the difference in the percent chance of overall survival for treatment with radiation therapy to percent chance of overall survival for treatment without radiation therapy to (C) the difference in the percent chance of overall survival for treatment with a combination of alkylating chemotherapy and radiation therapy to the percent chance of overall survival for treatment without the combination of alkylating chemotherapy and radiation therapy; and selecting treatment having the greatest difference. Accordingly, in some aspects, the method further comprises calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination, and selecting the treatment with the greatest difference in percent chance of overall survival with the treatment vs. without the treatment. Therefore, once a set of scores is determined and correlated with a % chance of survival upon a particular treatment, a physician may have sufficient information to decide the best course of treatment for the subject.

Treatment Type

As used herein, the term “treatment” is meant “therapeutic treatment” or “therapy” or “treatment regimen” or “treatment modality”. As used herein, the term “alkylating chemotherapy” refers to an anti-cancer treatment comprising use of any compound or molecule that adds an alkyl group to a base of DNA (e.g., guanine, adenine, cytosine, thymidine) to cause cancer cell DNA damage and ultimately cancer cell death. In exemplary aspects, the alkylating chemotherapy comprises a nitrogen mustard, such as, but not limited to, a cyclophosphamide, chlormethine, uramustine, melphalan, chlorambucil, ifosfamide, and bendamustine. In exemplary aspects, the alkylating chemotherapy comprises a nitrosourea, such as, for example, carmustine, lomustine, streptozocin. In exemplary aspects, the alkylating chemotherapy comprises an alkyl suofonate, e.g., busulfan. In exemplary aspects, the alkylating chemotherapy comprises a platinum-based compound, such as, e.g., cisplatin, carboplatin, dicycloplatin, eptaplatin, lobaplatin, miriplatin, nedaplatin, oxaliplatin, picoplatin, straplatin, triplatin tetranitrate. In exemplary aspects, the alkylating chemotherapy comprises procarbazine or altretamine. In exemplary aspects, the alkylating chemotherapy comprises temozolomide (TMZ), lomustine (CCNU), carmustine (BCNU), or any combination thereof.

As used herein, the term “radiation therapy” refers to an anti-cancer treatment comprising beams of intense energy, such as X-rays or protons, to kill cancer cells. In exemplary aspects, the radiation therapy comprises external beam radiation. In exemplary aspects, the radiation therapy comprises brachytherapy or systemic radiation. In exemplary aspects, the radiation therapy comprises three-dimensional conformal radiation, intensity-modulated radiation therapy, high-dose/low-dose rate brachytherapy, stereotactic body radiation therapy, or intensity-modulated proton therapy.

Reference Levels

In some aspects of the methods described herein, the expression level that is measured may be the same as a reference level, e.g., a control level or a cut off level or a threshold level, or may be increased or decreased relative to a reference level, e.g., control level or a cut off level or a threshold level. In some aspects, the reference level is that of a reference subject which may be a matched control of the same species, gender, ethnicity, age group, smoking status, BMI, current therapeutic regimen status, medical history, or a combination thereof, but differs from the subject being diagnosed or from whom a sample was obtained in that the reference does not suffer from the disease in question or is not at risk for the disease. Thus, in exemplary aspects, the reference expression level(s) of the gene(s), RNA, or protein(s) is/are level(s) of a subject known to not have a tumor. In alternative aspects, the reference expression level(s) of the gene(s), RNA, or protein(s) is/are level(s) of a subject known to have a tumor. In exemplary aspects, as further described herein, the measured level is compared to both a reference level of a subject known to not have a tumor and a reference level of a subject known to have a tumor. In exemplary aspects, the reference level is the mean of a population of expression levels for the corresponding gene. For example, the reference level for an MGMT expression level of the sample is the mean MGMT expression level among a population, the reference level for an CHGA expression level of the sample is the mean CHGA expression level among a population, the reference level for an MAPK8 expression level of the sample is the mean MAPK8 expression level among a population, the reference level for an GPRASP1 expression level of the sample is the mean GPRASP1 expression level among a population, the reference level for an FGF7 expression level of the sample is the mean FGF7 expression level among a population, or the reference level for an ATP6V0A2 expression level of the sample is the mean ATP6V0A2 expression level among a population. In some aspects, the reference level is the expression level of the corresponding gene (e.g., MGMT, GPRASP1, CHGA, MAPK8, ATP6V0A2, or FGF7) in a population of subjects having a tumor and untreated for the tumor (e.g., a population of untreated subjects with a tumor).

Relative to a reference level, the level that is measured or determined may be increased. As used herein, the term “increased” with respect to level (e.g., expression level) refers to any % increase above a reference level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a reference level.

Relative to a reference level, the level that is determined may be decreased. As used herein, the term “decreased” with respect to level (e.g., expression level) refers to any % decrease below a reference level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a reference level.

Samples

The samples of the methods of the present disclosure are samples obtained from a subject. In some embodiments, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In exemplary aspects, the sample is a tissue sample obtained by a biopsy or by surgical resection.

In exemplary aspects, the sample comprises a cell or cells of a tumor in the subject. In exemplary aspects, the tumor is glioblastoma. In some aspects, the cell of the tumor comprises mutations in EGFR, PTEN, and p53. In exemplary aspects, the present disclosure methods further comprise obtaining the sample from the subject.

Tumors

For purposes herein, the tumor may be any type of tumor, or group of abnormal cells that form lumps or growths, known in the art. The tumor may be cancerous (malignant) or non-cancerous (benign) or precancerous. The tumor may be a carcinoma, a sarcoma, a myeloma, a leukemia, a lymphoma, or a mixed type tumor. The carcinoma may be, for example, an adenocarcinoma or a squamous cell carcinoma. The sarcoma, in some aspects, is an osteosarcoma or osteogenic sarcoma, a chondrosarcoma, a leiomyosarcoma, a rhabdomyosarcoma, a mesothelial sarcoma or mesothelioma, a fibrosarcoma, an angiosarcoma or hemangioendothelioma, a liposarcoma, a glioma or astrocytoma, a myxosarcoma, a mesenchymous or mixed mesodermal tumor. The leukemia may be Myelogenous or granulocytic leukemia, Lymphatic, lymphocytic, or lymphoblastic leukemia, or Polycythemia vera or erythremia. The mixed type may be an adenosquamous carcinoma, mixed mesodermal tumor, carcinosarcoma, or teratocarcinoma. The tumor in certain instances is selected from the group consisting of: fibrosarcoma, myxosarcoma, chondrosarcoma, osteosarcoma, chordoma, malignant fibrous histiocytoma, hemangiosarcoma, angiosarcoma, lymphangiosarcoma, mesothelioma, plasmacytoma, multiple myeloma, Hodgkin lymphoma, Non-Hodgkin lymphoma, squamous cell carcinoma, epidermoid carcinoma, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma, neuroblastoma, medulloblastoma, malignant meningioma, malignant schwannoma, neurofibrosarcoma, parathyroid carcinoma, medullary carcinoma of thyroid, brochinam carcinooid, oat cell carcinoma, malignant pheochromocytoma, islet cell carcinoma, malignant carcinoid, malignant paraganglioma, melanoma, malignant schwannoma, Merkel cell neoplasm, cystosarcoma, Wilms tumor, and gonadal tumor. The tumor in some aspects is a tumor of any of: acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, uterine cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In exemplary aspects, the metastatic cancer is triple-negative breast cancer, pancreatic cancer, prostate cancer, or melanoma.

Subjects

In exemplary aspects, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less. In exemplary aspects, the subject has a tumor or cancer. The tumor or cancer may be any of those known in the art or described herein.

Additional Steps

With regard to the methods of the invention, the methods may include additional steps. For example, the method may include repeating one or more of the recited step(s) of the method. Accordingly, in exemplary aspects, the method comprises measuring a level of expression of a gene, an RNA, or a protein, in a sample obtained from a subject and re-measuring the level, e.g., at a different time point, for accuracy. In exemplary aspects, the method comprises obtaining the sample from the subject. In exemplary embodiments, more than one sample is obtained from the subject. In exemplary embodiments, 2, 3, 4, 5, 6, 7, 8, 9, 10, or more samples are obtained from the subject, each sample obtained at a different point in time. In exemplary aspects, a sample is obtained from the subject once a week, once a month, 2× per month, 3× per month, 4× per month or more frequently. In exemplary aspects, a sample is obtained from the subject once a year, once a quarter, 2× per year, 3× per year, 4× per year or more frequently. In exemplary aspects, a sample is obtained on a regular basis based on the analysis of a first sample. In exemplary aspects, a sample is obtained on a regular basis until a pre-determined goal is met. In exemplary aspects, the pre-determined goal is the determination of the subject as exhibiting a complete therapeutic response to a treatment, e.g., alkylating chemotherapy, radiation therapy, combination thereof. In exemplary instances, the methods comprises monitoring the subject during treatment.

In exemplary aspects, the method comprises measuring an expression level for every sample obtained. In exemplary aspects, the expression level is measured within 1, 4, 6, 8, 12, 16, or 24 hours of obtaining the sample. In exemplary aspects, the sample is cryopreserved and expression of the sample is determined at a later time. In exemplary instances, the sample is formalin fixed and paraffin-embedded and expression of the sample is determined at a later time.

In exemplary aspects, the methods comprise processing the sample for measurement of expression. For example, the methods may comprise RNA isolation from cells of the scaffold. The methods may comprises homogenizing in a Trizol reagent for RNA isolation or in a detergent for protein isolation. In exemplary aspects, the method comprises synthesizing cDNA based on the isolated RNA.

In exemplary aspects, the method comprises measuring an expression level of the sample in more than one way. In exemplary instances, the methods comprise measuring expression using a gene chip and an ELISA or other immunoassay. In exemplary instances, the methods comprise measuring expression levels of one or more housekeeping genes and comparing the measured levels of genes to housekeeping genes. In exemplary aspects, the method comprises normalizing the expression level data to expression levels of one or more housekeeping genes.

In exemplary aspects, the methods comprise administering treatment to a subject. Thus, methods of treatment are provided, as described below.

Methods of Treatment

The present disclosure provides methods of treating a tumor in a subject. In exemplary embodiments, the subject is one from whom a sample comprising a cell or cells from the tumor was obtained and the expression level of MGMT, GPRASP1, CHGA, MAPK8, ATP6V0A2, FGF7, or any combination thereof, of the sample was measured, and the method comprises administering to the subject (i) an alkylating chemotherapy, (ii) a radiation therapy, or (iii) a combination of alkylating chemotherapy and radiation therapy, in an amount effective to treat the tumor, depending on the measured expression level. In exemplary embodiments, the expression levels of (A) MGMT and GPRASP1 or (B) CHGA and MAPK8 were measured, and, optionally, the method comprises administering to the subject: (A) an alkylating chemotherapy, when the measured expression levels of MGMT and GPRASP1 in the sample were decreased relative to a reference level, or (B) (B) radiation therapy, when the measured expression levels of CHGA and MAPK8 in the sample were increased relative to a reference level. In alternative or additional embodiments, the expression levels of MGMT, GPRASP1, CHGA, MAPK8, ATP6V0A2, and FGF7 were measured, and, optionally, the method comprises administering to the subject a combination comprising alkylating chemotherapy and radiation therapy, when the measured expression levels of MGMT, GPRASP1, ATP6V0A2, and FGF7 in the sample were decreased, relative to a reference level, and/or the measured expression levels of CHGA and MAPK8 in the sample were increased, relative to a reference level.

In exemplary embodiments of the presently disclosed method of treating a tumor, the subject has a decreased expression level of MGMT or GPRASP1, or both, relative to a reference level. In exemplary instances, the method comprises administering to the subject an alkylating chemotherapy in an amount effective to treat the tumor.

In exemplary embodiments of the presently disclosed method of treating a tumor, the subject has an increased expression level of CHGA or MAPK8, or both, relative to a reference level. In exemplary instances, the method comprises administering to the subject a radiation therapy in an amount effective to treat the tumor.

In exemplary embodiments of the presently disclosed method of treating a tumor, the subject has (A) a decreased expression level of MGMT, GPRASP1, ATP6V0A2, FGF7, or any combination thereof, relative to a reference level, or (B) an increased expression level of CHGA or MAPK8, or both, relative to a reference level, or (C) both (A) and (B). In exemplary aspects, the method comprises administering to the subject a combination comprising an alkylating chemotherapy and a radiation therapy in an amount effective to treat the tumor.

In exemplary aspects, the method of treating a tumor in a subject comprises the step of measuring the expression level. Accordingly, in exemplary embodiments, the method comprises (a) measuring the expression level of MGMT, GPRASP1, CHGA, MAPK8, ATP6V0A2, FGF7, or any combination thereof, in a sample comprising a cell or cells from the tumor, and (b) administering to the subject: (i) an alkylating chemotherapy, (ii) a radiation therapy, or (iii) a combination of alkylating chemotherapy and radiation therapy, in an amount effective to treat the tumor, depending on the measured expression level. In some embodiments, the method comprises measuring the expression levels of (A) MGMT and GPRASP1 or (B) CHGA and MAPK8, and, optionally, the method comprises administering to the subject: (A) an alkylating chemotherapy, when the measured expression levels of MGMT and GPRASP1 in the sample are decreased relative to a reference level, or (B) radiation therapy, when the measured expression levels of CHGA and MAPK8 in the sample are increased relative to a reference level. In alternative or additional aspects, the method comprises measuring the expression levels of MGMT, GPRASP1, CHGA, MAPK8, ATP6V0A2, and FGF7, and optionally, the method comprises administering to the subject a combination comprising alkylating chemotherapy and radiation therapy, when the measured expression levels of MGMT, GPRASP1, ATP6V0A2, and FGF7 in the sample are decreased, relative to a reference level, and/or the measured expression levels of CHGA and MAPK8 in the sample are increased, relative to a reference level.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a tumor of the present disclosure can provide any amount or any level of treatment. Furthermore, the treatment provided by the method of the present disclosure can include treatment of one or more conditions or symptoms or signs of the tumor being treated. Also, the treatment provided by the methods of the present disclosure can encompass slowing the progression of tumor growth or preventing the growth of new tumors. For example, the methods can treat the tumor by virtue of preventing or slowing tumor metastasis, enhancing an immune response against the tumor, reducing metastasis of tumor cells, increasing cell death of tumor cells, and the like. In exemplary aspects, the methods treat by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In exemplary aspects, the methods treat by way increasing the survival of the subject. In some aspects, the method of treating encompasses a method of prophylactically treating (i.e., preventing) or delaying the onset of a disease. In aspects, the disease is cancer, e.g., metastatic cancer. Because cancer is lethal due to its nature of becoming metastatic cancer, delaying the onset of metastatic disease may effectively increase the survival of the subject. Accordingly, the presently disclosed methods of treatment in some aspects encompass methods of increasing the survival of a subject with a tumor.

Kits

The present disclosure also provides kits comprising one or more binding agents specific for MGMT, GPRASP1, CHGA, MAPK8, ATP6V0A2, or FGF7. With regard to the foregoing, the binding agent in some aspects is an antibody, antigen binding fragment, an aptamer, a protein or peptide substrate, or a nucleic acid probe. In exemplary aspects, the binding agent is an antibody, or antigen-binding fragment thereof. In exemplary aspects, the binding agent is a nucleic acid probe specific for MGMT, GPRASP1, CHGA, MAPK8, ATP6V0A2, or FGF7, or a complement thereof. Such binding agents are known in the art. In some aspects, the kit comprises a collection of binding agents, e.g., a collection of antibodies, a collection of nucleic acid probes, each binding agent of which specifically binds to genes or nucleic acids encoding the marker. In some aspects, the collection of nucleic acid probes is formatted in an array on a solid support, e.g., a gene chip. In some aspects, the kit comprises a collection of antibodies which specifically bind to a marker. In some aspects, the kit comprises a multi-well microtiter plate, wherein each well comprises an antibody having a specificity which is unique to the antibodies of the other wells. In some aspects, the kit comprises a collection of substrates which specifically react with a marker. In some aspects, the kit comprises a multi-well microtiter plate, wherein each well comprises a substrate having a specificity which is unique to the substrates of the other wells.

In some aspects, the kits further comprises instructions for use. In some aspects, the instructions are provided as a paper copy of instructions, an electronic copy of instructions, e.g., a compact disc, a flash drive, or other electronic medium. In some aspects, the instructions are provided by way of providing directions to an internet site at which the instructions may be accessed by the user.

In some aspects, the kits further comprise a unit for a collecting a biological sample, e.g., any of the samples described herein, of the subject. In some aspects, the unit for collecting a sample is a vial, a beaker, a tube, a microtiter plate, a petri dish, and the like.

Systems, Computer-Readable Storage Media, and Methods Implemented by a Computer Processor

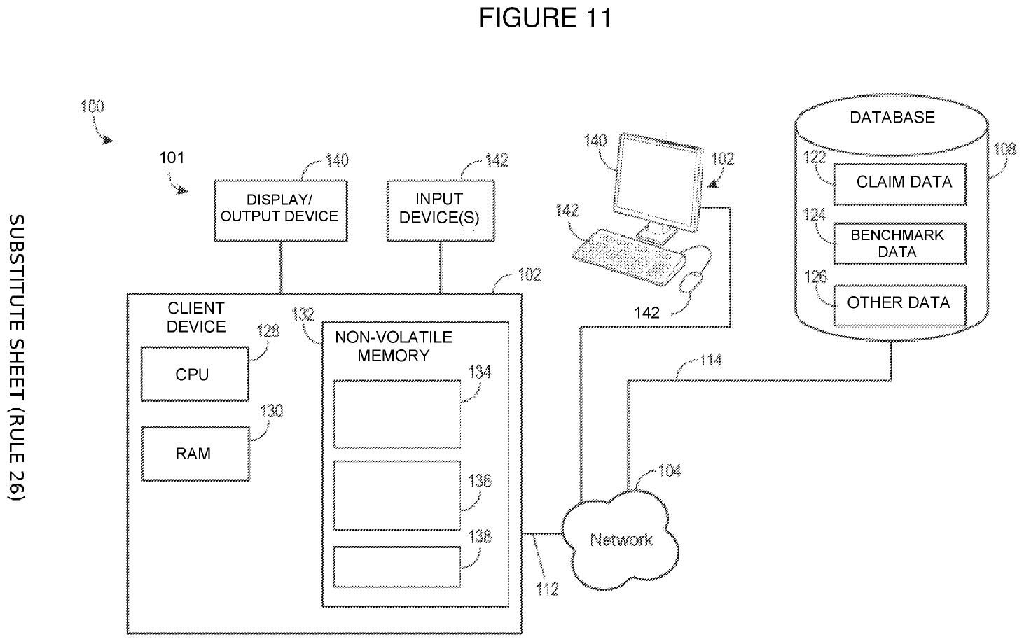

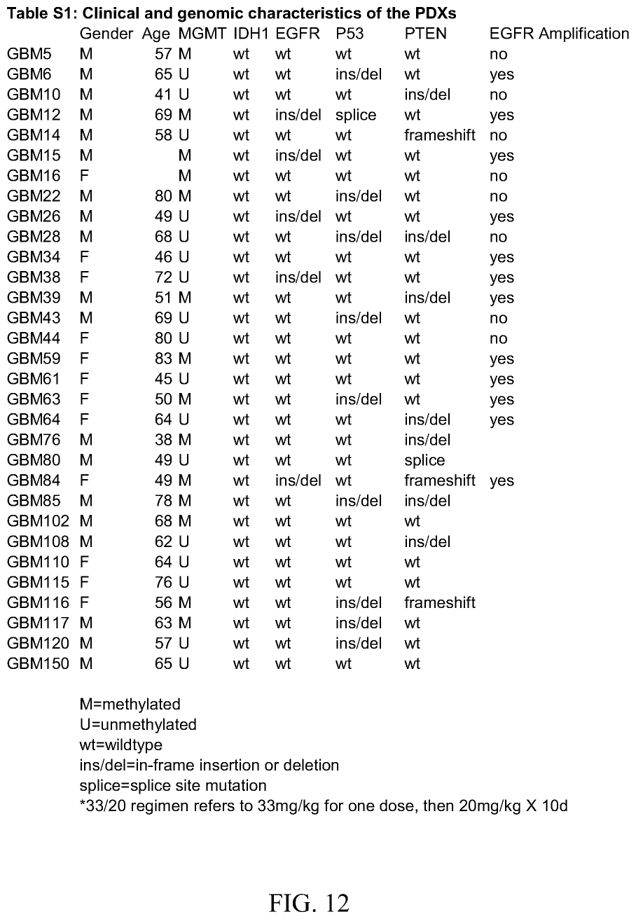

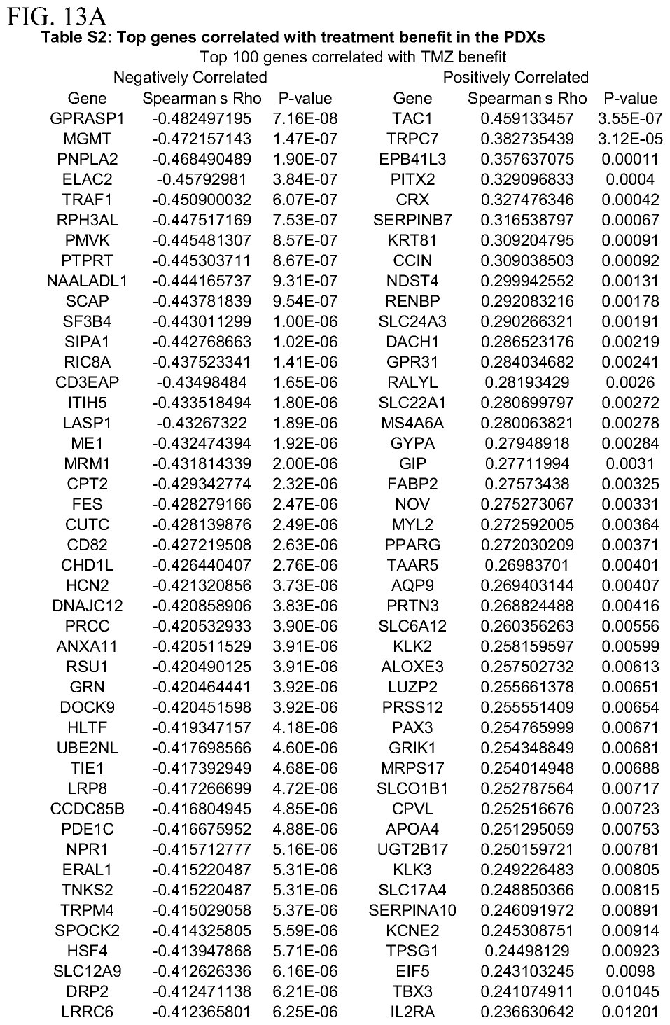

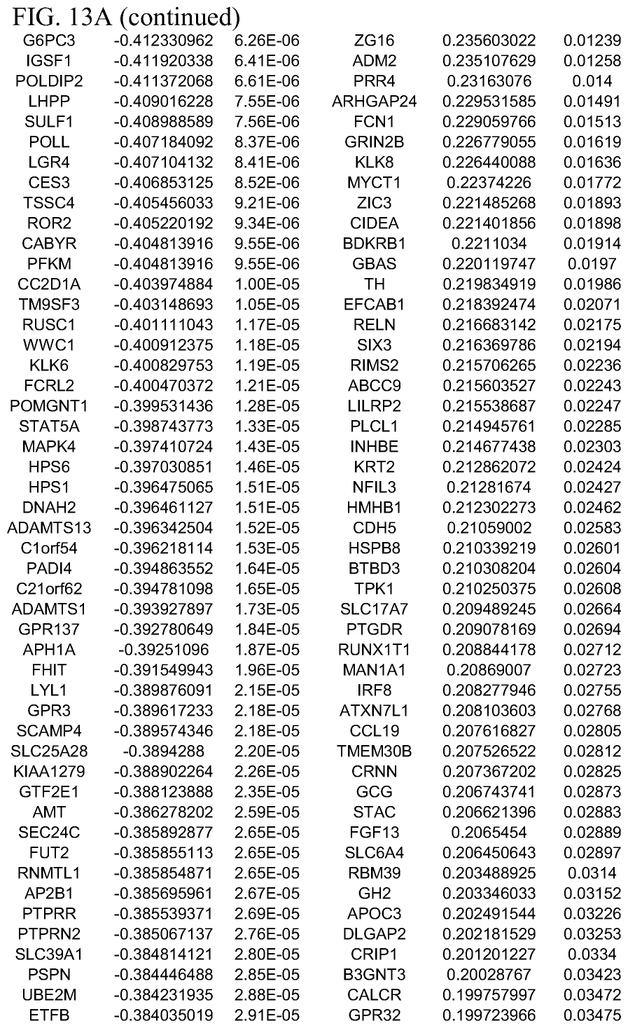

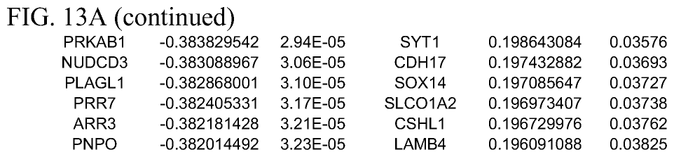

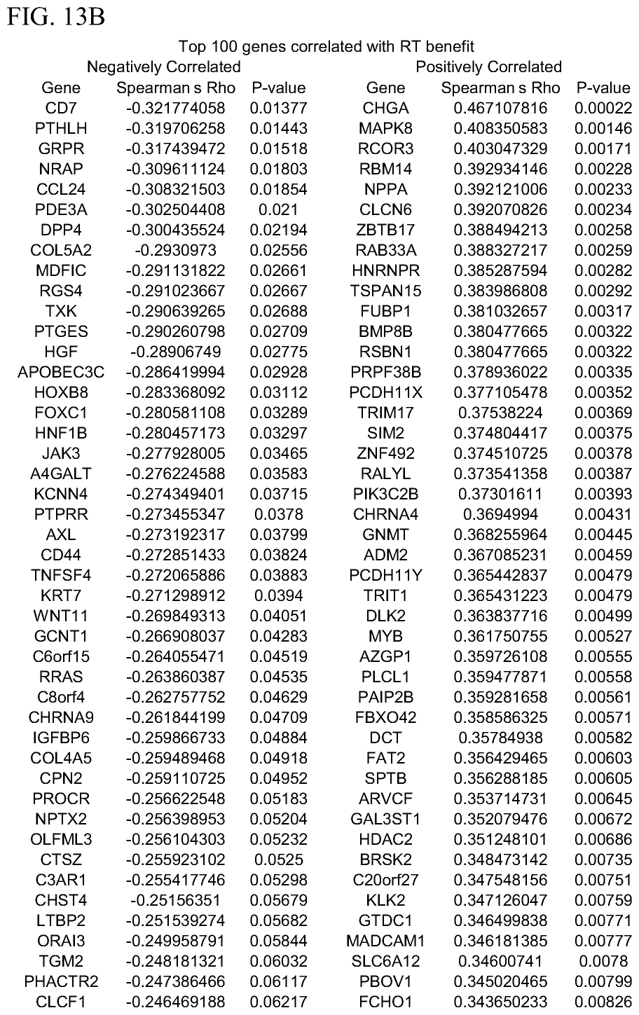

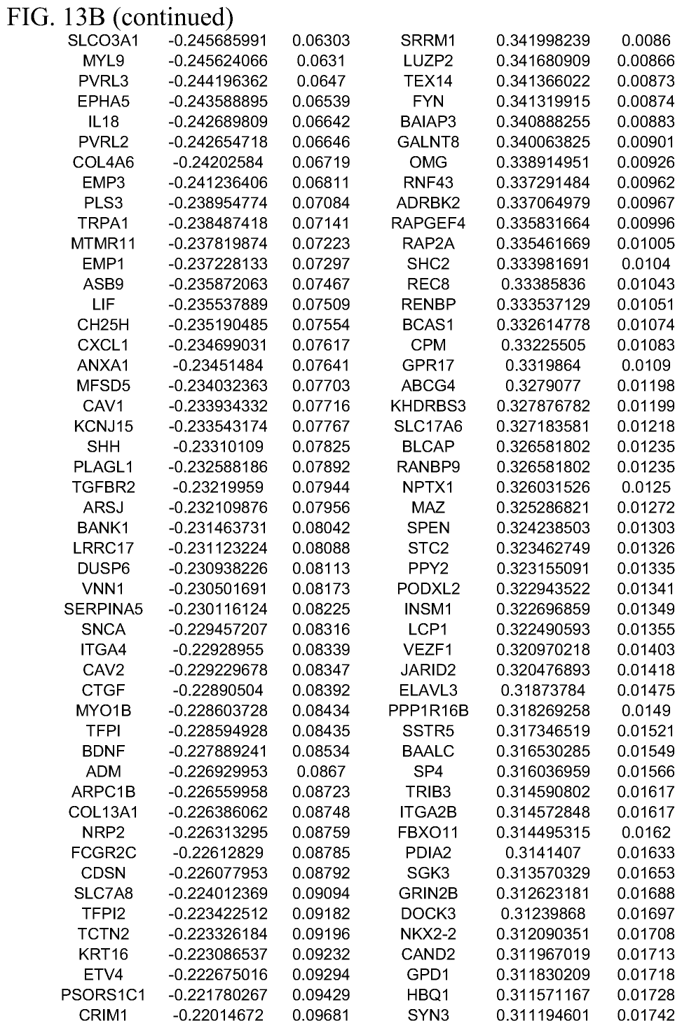

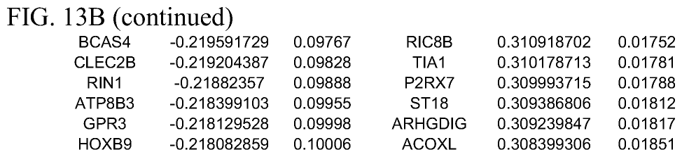

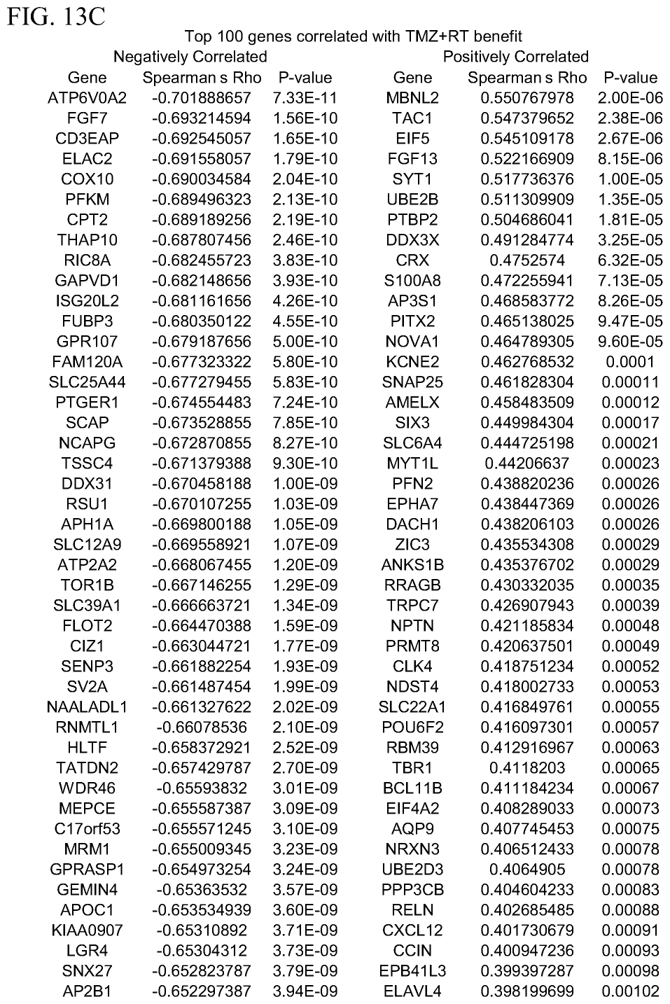

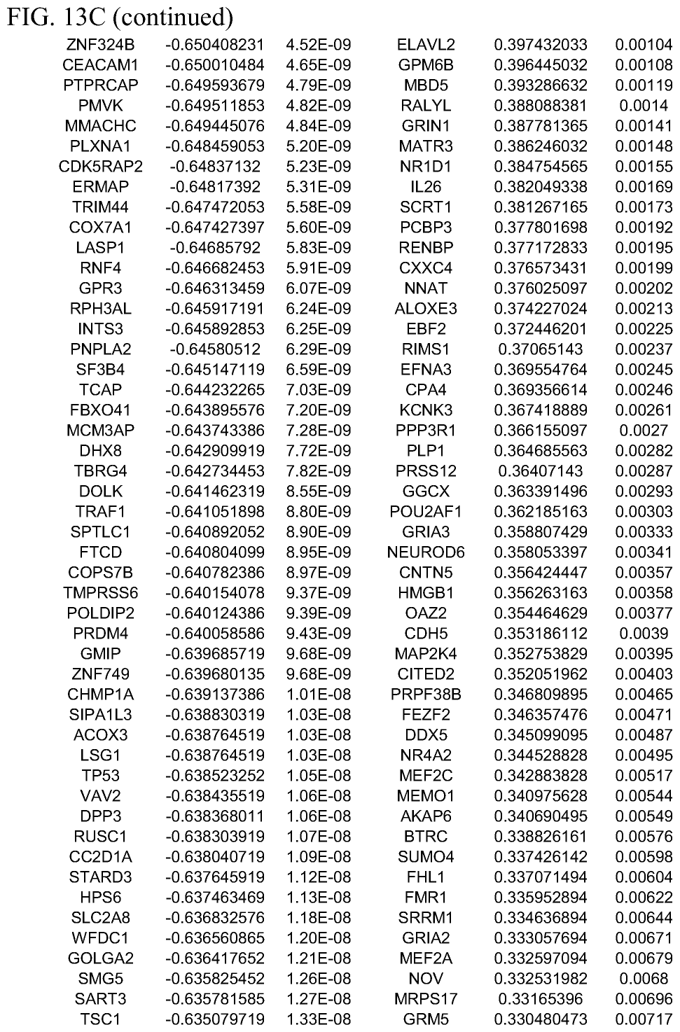

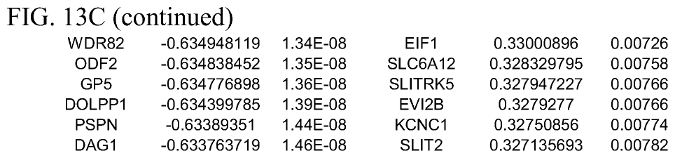

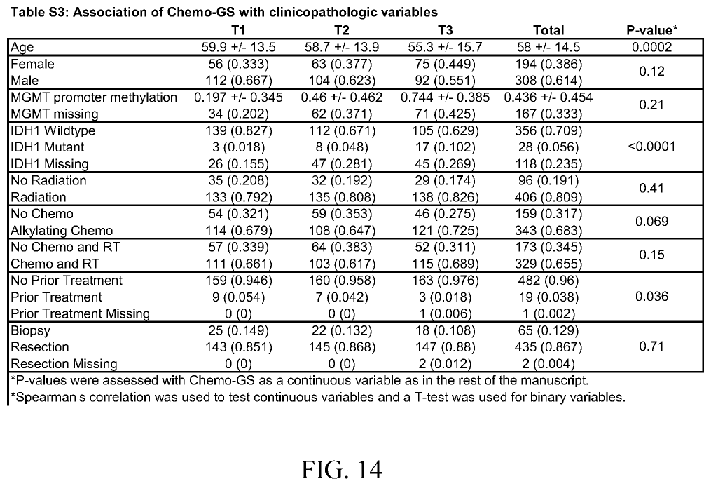

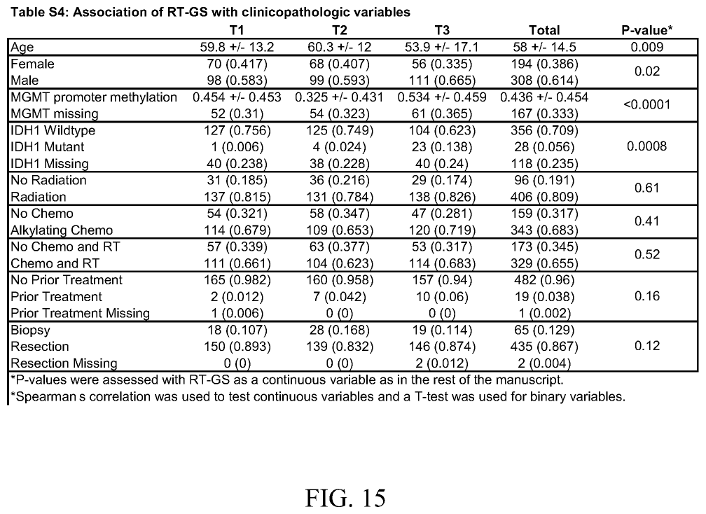

FIG. 11 illustrates an exemplary embodiment 101 of a system 100 for assessing a subject's metastatic potential. Generally, the system 100 may include one or more client devices 102, a network 104, and a database 108. Each client device 102 may be communicatively coupled to the network 104 by one or more wired or wireless network connections 112, which may be, for example, a connection complying with a standard such as one of the IEEE 802.11 standards (“Wi-Fi”), the Ethernet standard, or any other appropriate network connection. Similarly, the database 108 may be communicatively coupled to the network 104 via one or more connections 114. (Of course, the database could alternatively be internal to one or more of the client devices 102.) The database 108 may store data related to the expression profiles for a variety of subjects, including, but not limited to, data of a sample obtained from a subject, data of a reference or control population, etc.

As will be understood, the network 104 may be a local area network (LAN) or a wide-area network (WAN). That is, network 104 may include only local (e.g., intra-organization) connections or, alternatively, the network 104 may include connections extending beyond the organization and onto one or more public networks (e.g., the Internet). In some embodiments, for example, the client device 102 and the database 108 may be within the network operated by a single company (Company A). In other embodiments, for example, the client device(s) 102 may be on a network operated by Company A, while the database 108 may be on a network operated by a second company (Company B), and the networks of Company A and Company B may be coupled by a third network such as, for example, the Internet.

Referring still to FIG. 11, the client device 102 includes a processor 128 (CPU), a RAM 130, and a non-volatile memory 132. The non-volatile memory 132 may be any appropriate memory device including, by way of example and not limitation, a magnetic disk (e.g., a hard disk drive), a solid state drive (e.g., a flash memory), etc. Additionally, it will be understood that, at least with regard to FIG. 11, the database 108 need not be separate from the client device 102. Instead, in some embodiments, the database 108 is part of the non-volatile memory 132 and the data 122, 124, 126 may be stored as data within the memory 132. For example, the data 122 may be included as data in a spreadsheet file stored in the memory 132, instead of as data in the database 108. In addition to storing the records of the database 108 (in some embodiments), the memory 132 stores program data and other data necessary to analyze data of one or more sample and/or control populations, etc. For example, in an embodiment, the memory 132 stores a first routine 134, a second routine 136, and a third routine 138. The first routine 134 may receive data values related to a measured expression level of a gene, RNA, or protein of a sample obtained from a scaffold implanted in a test subject, and may process the data values received by the routine 134 through an algorithm to obtain a score. The second routine 136 may computer one or more statistical parameters of the data collected by the first routine 134, such as determining a mean value, a standard deviation value, etc. Additionally and/or alternatively, the second routine 136 may plot a score on a graphical or numerical output. Regardless, each of the routines is executable by the processor 128 and comprises a series of compiled or compilable machine-readable instructions stored in the memory 132. Additionally, the memory 132 may store generated reports or records of data output by one of the routines 134 or 136. Alternatively, the reports or records may be output to the database 108. One or more display/output devices 140 (e.g., printer, display, etc.) and one or more input devices 142 (e.g., mouse, keyboard, tablet, touch-sensitive interface, etc.) may also be coupled to the client device 102, as is generally known.

As will be understood, although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

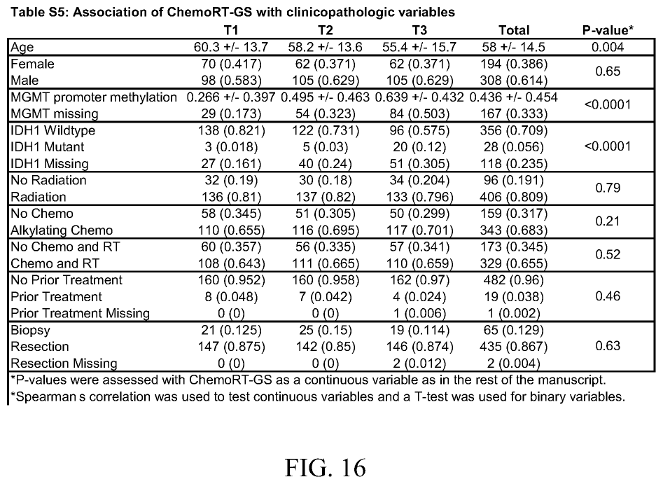

For example, the network 104 may include but is not limited to any combination of a LAN, a MAN, a WAN, a mobile, a wired or wireless network, a private network, or a virtual private network. Moreover, while only two clients 102 are illustrated in FIG. 16 to simplify and clarify the description, it is understood that any number of client computers are supported and can be in communication with one or more servers (not shown).

Additionally, certain embodiments are described herein as including logic or a number of routines. Routines may constitute either software routines (e.g., code embodied on a machine-readable medium or in a transmission signal) or hardware routines. A hardware routine is tangible unit capable of performing certain operations and may be configured or arranged in a certain manner. In example embodiments, one or more computer systems (e.g., a standalone, client or server computer system) or one or more hardware routines of a computer system (e.g., a processor or a group of processors) may be configured by software (e.g., an application or application portion) as a hardware routine that operates to perform certain operations as described herein.

Similarly, the methods or routines described herein may be at least partially processor-implemented. For example, at least some of the operations of a method may be performed by one or processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description should be read to include one or at least one and the singular also includes the plural unless it is obvious that it is meant otherwise.

Upon reading this disclosure, those of skill in the art will appreciate still additional alternative structural and functional designs for a system and a process for identifying terminal road segments through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended listing of exemplary embodiments.

The present disclosure provides systems comprising: a processor; a memory device coupled to the processor, and machine readable instructions stored on the memory device. In exemplary embodiments, the machine readable instructions that, when executed by the processor, cause the processor to center and scale measured expression levels of MGMT, GPRASP1, CHGA, MAPK8, ATP6V0A2, or FGF7 relative to a reference level, and optionally, assign a score using an equation, and subsequently determining a percent chance overall survival. In exemplary aspects, the machine readable instructions that, when executed by the processor, cause the processor to
(i) receive a measured expression level of a sample obtained from a subject with a tumor for each of MGMT, GPRASP1, CHGA, MAPK8, ATP6V0A2, or FGF7;
(ii) receive a plurality of data values, each data value is a measured expression level of MGMT, GPRASP1, CHGA, MAPK8, ATP6V0A2, or FGF7 among a population of subjects;
(iii) for each of MGMT, GPRASP1, CHGA, MAPK8, ATP6V0A2, and FGF7, calculate a mean and a standard deviation of the data values received in step (ii);
(iv) subtract the mean from the corresponding measured expression level to obtain a mean-adjusted expression level for each of MGMT, GPRASP1, CHGA, MAPK8, ATP6V0A2, and FGF7,
(v) divide each mean-adjusted expression level by the corresponding standard deviation to obtain a centered and scaled expression level;
(vi) calculate an alkylating chemotherapy score using Equation 1:

$$\frac{( - 1 ) ( GPRASP 1 ) + ( - 1 ) ( MGMT )}{2} \qquad ( Equation 1 )$$

(B) a radiation therapy score using Equation 2:

$$\frac{( CHGA ) + ( MAPK 8 )}{2} ; \qquad ( Equation 2 )$$

and/or
(C) a combination alkylating chemotherapy/radiation therapy score by using Equation 3:

$$\frac{\begin{matrix}( - 1 ) ( GPRASP 1 ) + ( - 1 ) ( MGMT ) + C H GA + \\ MAPK 8 + ( - 1 ) ( ATP 6 V 0 A 2 ) + ( - 1 ) ( FGF 7 )\end{matrix}}{6} \qquad ( Equation 3 )$$

wherein “MGMT”, “GPRASP1”, “CHGA”, “MAPK8”, “ATP6V0A2”, and “FGF7” is the centered and scaled expression level as determined in (v);
(vii) use the alkylating chemotherapy score to determine a percent chance of overall survival for treatment with alkylating chemotherapy, the radiation therapy score to determine percent chance of overall survival for treatment with radiation therapy, and combination alkylating chemotherapy/radiation therapy score to determine percent chance of overall survival for treatment with a combination of alkylating chemotherapy and radiation therapy; and calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination,
(viii) select the treatment with the greatest difference in percent chance of overall survival with the treatment vs. without the treatment.

Also provided herein are computer-readable storage media having stored thereon machine-readable instructions executable by a processor. In exemplary aspects, the machine-readable instructions comprise instructions for centering and scaling measured expression levels of MGMT, GPRASP1, CHGA, MAPK8, ATP6V0A2, or FGF7 relative to a reference level, and optionally, instructions for assigning a score using an equation, and instructions for subsequently determining a percent chance overall survival. In exemplary embodiments, the instructions comprise:
(i) instructions for receiving a measured expression level of a sample obtained from a subject with a tumor for each of MGMT, GPRASP1, CHGA, MAPK8, ATP6V0A2, or FGF7;
(ii) instructions for receiving a plurality of data values, each data value is a measured expression level of MGMT, GPRASP1, CHGA, MAPK8, ATP6V0A2, or FGF7 among a population of subjects;
(iii) for each of MGMT, GPRASP1, CHGA, MAPK8, ATP6V0A2, and FGF7, instructions for calculating a mean and a standard deviation of the data values received in step (ii);
(iv) instructions for subtracting the mean from the corresponding measured expression level to obtain a mean-adjusted expression level for each of MGMT, GPRASP1, CHGA, MAPK8, ATP6V0A2, and FGF7,
(v) instructions for dividing each mean-adjusted expression level by the corresponding standard deviation to obtain a centered and scaled expression level;
(vi) instructions for calculating
   (A) an alkylating chemotherapy score using Equation 1:

$$\frac{( - 1 ) ( GPRASP 1 ) + ( - 1 ) ( MGMT )}{2} \qquad ( Equation 1 )$$

(B) a radiation therapy score using Equation 2:

$$\frac{( CHGA ) + ( MAPK 8 )}{2} ; \qquad ( Equation 2 )$$

and/or
(C) a combination alkylating chemotherapy/radiation therapy score by using Equation 3:

$$\frac{\begin{matrix}( - 1 ) ( GPRASP 1 ) + ( - 1 ) ( MGMT ) + C H GA + \\ MAPK 8 + ( - 1 ) ( ATP 6 V 0 A 2 ) + ( - 1 ) ( FGF 7 )\end{matrix}}{6} \qquad ( Equation 3 )$$

wherein “MGMT”, “GPRASP1”, “CHGA”, “MAPK8”, “ATP6V0A2”, and “FGF7” is the centered and scaled expression level as determined in (v);
(vii) instructions for using the alkylating chemotherapy score to determine a percent chance of overall survival for treatment with alkylating chemotherapy, the radiation therapy score to determine percent chance of overall survival for treatment with radiation therapy, and combination alkylating chemotherapy/radiation therapy score to determine percent chance of overall survival for treatment with a combination of alkylating chemotherapy and radiation therapy; calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination, and
(viii) instructions for selecting the treatment with the greatest difference in percent chance of overall survival with the treatment vs. without the treatment.

Further provided herein are methods implemented by a processor in a computer. In exemplary embodiments, the method comprises the steps of centering and scaling measured expression levels of MGMT, GPRASP1, CHGA, MAPK8, ATP6V0A2, or FGF7 relative to a reference level, and optionally, assigning a score using an equation, and subsequently determining a percent chance overall survival. In exemplary embodiments, the method comprises the steps of:
(i) receiving a measured expression level of a sample obtained from a subject with a tumor for each of MGMT, GPRASP1, CHGA, MAPK8, ATP6V0A2, or FGF7;
(ii) receiving a plurality of data values, each data value is a measured expression level of MGMT, GPRASP1, CHGA, MAPK8, ATP6V0A2, or FGF7 among a population of subjects;
(iii) for each of MGMT, GPRASP1, CHGA, MAPK8, ATP6V0A2, and FGF7, calculating a mean and a standard deviation of the data values received in step (ii);
(iv) subtracting the mean from the corresponding measured expression level to obtain a mean-adjusted expression level for each of MGMT, GPRASP1, CHGA, MAPK8, ATP6V0A2, and FGF7,
(v) dividing each mean-adjusted expression level by the corresponding standard deviation to obtain a centered and scaled expression level;
(vi) calculating an alkylating chemotherapy score using Equation 1:

$$\frac{( - 1 ) ( GPRASP 1 ) + ( - 1 ) ( MGMT )}{2} \qquad ( Equation 1 )$$

(B) a radiation therapy score using Equation 2:

$$\frac{( CHGA ) + ( MAPK 8 )}{2} ; \qquad ( Equation 2 )$$

and/or
(C) a combination alkylating chemotherapy/radiation therapy score by using Equation 3:

$$\frac{\begin{matrix}( - 1 ) ( GPRASP 1 ) + ( - 1 ) ( MGMT ) + C H GA + \\ MAPK 8 + ( - 1 ) [ A T P 6 V 0 A 2 ) + ( - 1 ) ( FGF 7 )\end{matrix}}{6} \qquad ( Equation 3 )$$

wherein “MGMT”, “GPRASP1”, “CHGA”, “MAPK8”, “ATP6V0A2”, and “FGF7” is the centered and scaled expression level as determined in (v);
(vii) using the alkylating chemotherapy score to determine a percent chance of overall survival for treatment with alkylating chemotherapy, the radiation therapy score to determine percent chance of overall survival for treatment with radiation therapy, and combination alkylating chemotherapy/radiation therapy score to determine percent chance of overall survival for treatment with a combination of alkylating chemotherapy and radiation therapy; and calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination,
(viii) selecting the treatment with the greatest difference in percent chance of overall survival with the treatment vs. without the treatment.

With regard to the presently disclosed systems, media, and methods, in some embodiments, the machine readable instructions that, when executed by the processor, cause the processor to determine a score for at least two or three of MGMT, GPRASP1, CHGA, MAPK8, ATP6V0A2, and FGF7 for the subject. In some instances, the machine readable instructions that, when executed by the processor, cause the processor to determine a score for at least four or five of MGMT, GPRASP1, CHGA, MAPK8, ATP6V0A2, and FGF7 for the subject. In other aspects, the machine readable instructions that, when executed by the processor, cause the processor to determine a score for all six of MGMT, GPRASP1, CHGA, MAPK8, ATP6V0A2, and FGF7 for the subject. Also, in some aspects, each subject of the population of (i) is a subject with a tumor which has been untreated for the tumor. Alternatively, each subject of the population of (i) is a subject with a tumor treated with an alkylating chemotherapy. Alternatively, each subject of the population of (i) is a subject with a tumor treated with radiation. Alternatively, each subject of the population of (i) is a subject with a tumor treated with a combination comprising an alkylating chemotherapy and radiation therapy.

The following examples are given merely to illustrate the present invention and not in any way to limit its scope.

### EXAMPLES

### Example 1

This example describes methods used in Example 2.

Patient Derived Xenografts

Data on 31 orthotopic GBM PDXs with baseline RNAseq data were obtained from the Mayo Clinic PDX National Resource. Clinical characteristics and genomic data on these PDXs are summarized in Table S1 and publicly available at www.mayo.edu/research/labs/translational-neuro-oncology/mayo-clinic-brain-tumor-patient-derived-xenograft-national-resource. These PDX models have been used to evaluate various treatments, including radiation (RT, n=67 experiments on 31 PDXs), temozolomide (TMZ, n=137 experiments on 31 PDXs), and radiation with TMZ (RT+TMZ, n=79 experiments on 29 PDXs), as previously described^{15, 16}. Gene expression was log₂ transformed, centered, and scaled¹⁷ using the “scale” function in R. Additional details on the analyses in the PDXs can be found in the supplemental methods.

TCGA

TCGA¹⁸ gene expression data for GBM was downloaded from the UCSC cancer browser¹⁹. Affymetrix U133A microarray data was selected for analysis, rather than RNAseq data, as microarray data was available on more samples. Expression, treatment, and outcomes were available for 502 patients. Gene expression was centered and scaled as above. Patients were classified as having received chemotherapy if they had received temozolomide or other alkylating chemotherapy during their treatment course and were classified as having received ChemoRT if they had received both modalities of chemotherapy and radiation. Additional details on the analyses in TCGA can be found in the supplemental methods.

Gene Signature Development

The primary endpoint for each PDX experiment was the ratio of survival time with treatment relative to survival time without treatment. Spearman's correlation was calculated for each gene to this ratio. Gene expression signatures for treatment response were developed using the genes with the highest absolute correlation coefficients. A score was created from the top genes by averaging^{20,21} their expression. For any gene selected for signature development with a negative correlation coefficient, the expression was multiplied by −1 such that a higher value always corresponded with increased benefit from treatment. All model development was performed exclusively in the PDXs. To identify biological pathways associated with treatment response, we used Gene Set Enrichment Analysis²² (GSEA). Additional details on the PDXs, RNAseq, and GSEA can be found in the supplemental methods.

Clinical Validation of Gene Signatures

The primary endpoint in TCGA was overall survival. Once signatures were defined in the PDX data, they were independently validated in TCGA without further modification. To assess for predictive potential, Cox regression was performed to test the interaction between the signatures and treatment²³. Multivariate interaction analysis (MVA) was used to adjust for treatment selection bias as previously described²⁴. Gene signatures, MGMT promoter methylation, and gene expression were treated as continuous variables in Cox regression. This allowed the results to be comparable to each other and is also suggested by Janes et al²³ for treatment selection biomarker evaluation. Therefore, all statistical inference was performed using gene signatures as continuous variables. Continuous variables are categorized into tertiles within Kaplan-Meier curves only for the purposes of visualization within the main text. The pre-specified analyses were the assessments of the three treatment signatures, MGMT promoter methylation, and MGMT expression for treatment benefit. P-values<0.05 were considered significant.

### Example 2

This example demonstrates the development and validation of xenograft-based platform-independent gene signatures that predict response to alkylating chemotherapy, radiation, and combination therapy in patients with glioblastoma.

In the first study of its kind, we performed RNAseq on a large cohort of GBM PDXs at baseline. We treated these PDXs with RT, TMZ or RT+TMZ and developed gene signatures (GS) predicting treatment response (termed RT-GS, Chemo-GS, and ChemoRT-GS). We then independently validated the gene signatures in The Cancer Genome Atlas (TCGA) GBM cohort to assess the clinical performance of the GS as predictive biomarkers, and compared our results to MGMT promoter methylation and gene expression.

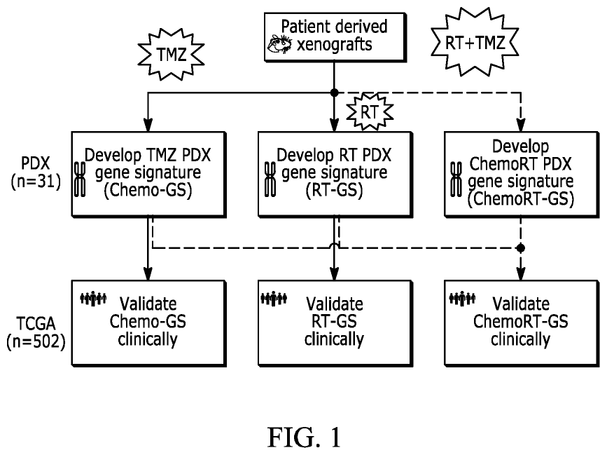

The overall study schema is depicted in FIG. 1. We utilized the gene expression and treatment response data from the PDXs to develop gene signatures for RT, chemotherapy, and ChemoRT response, which were then independently validated in TCGA.

Patient Derived Xenografts

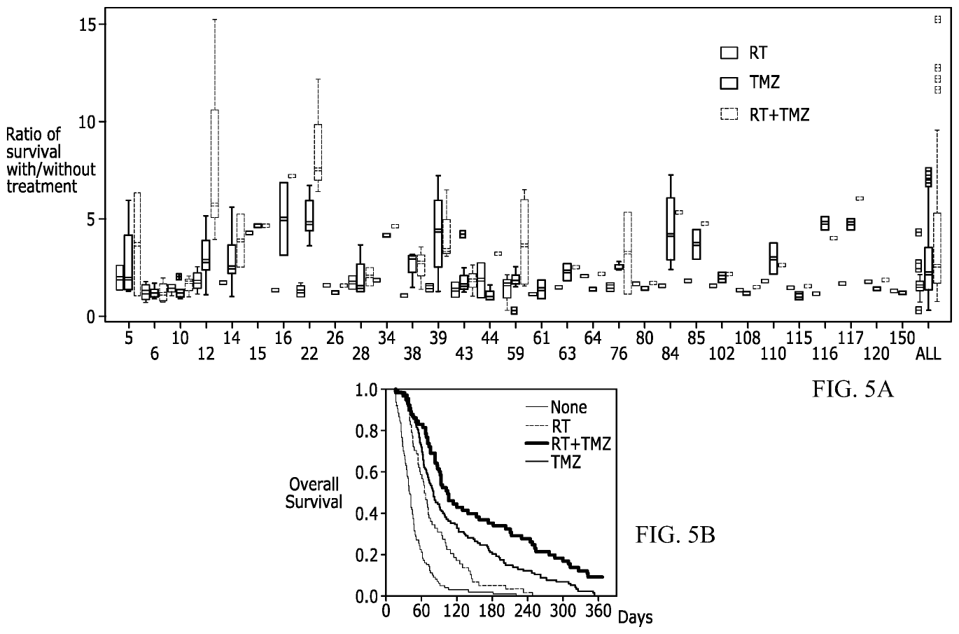

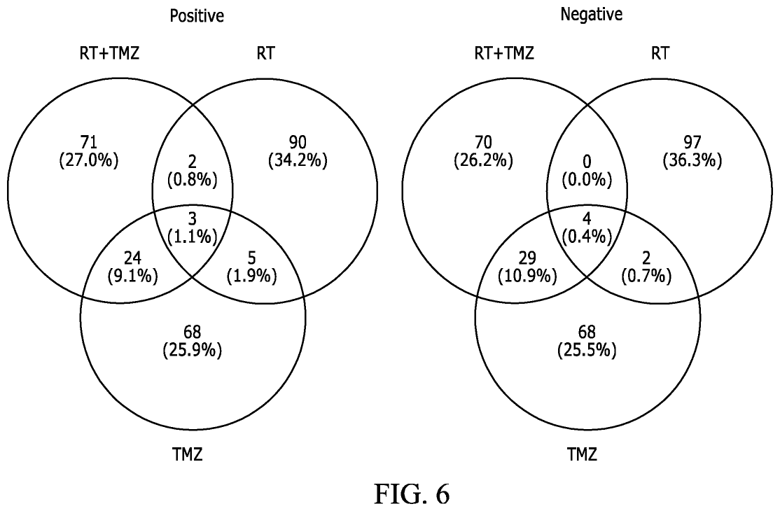

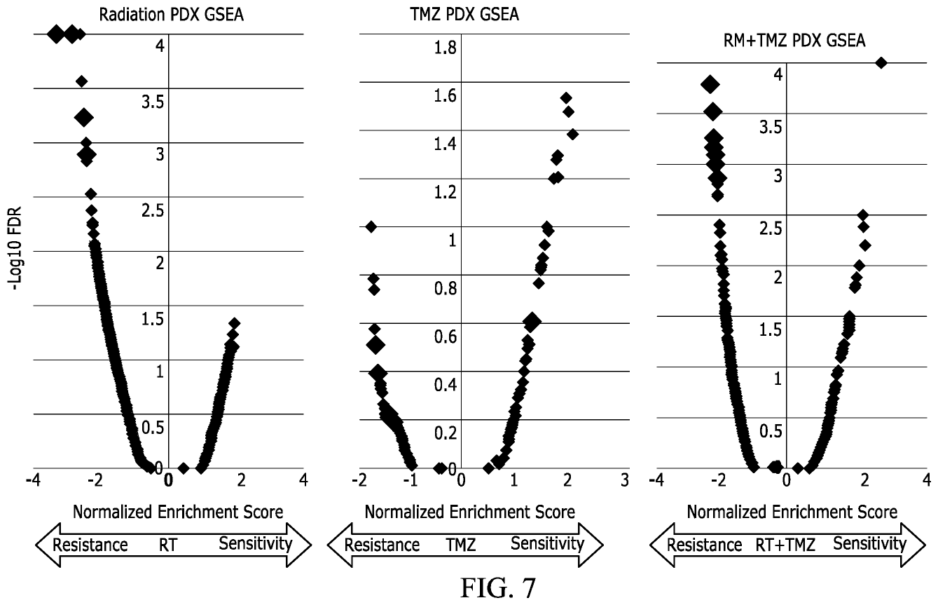

The PDXs recapitulated the heterogeneity of human GBMs. 61% were from male patients and 39% from females. The median age at diagnosis was 63 years, with a range from 38 to 83 years. MGMT promoter methylation occurred in 45% of samples³. All PDXs were IDH1 wild-type, but mutations in EGFR, PTEN, and P53 were all present¹⁸. Clinical and molecular characteristics are further summarized in Table S1, with all data publicly available online at the Mayo Clinic PDX National Resource website. Treatment benefit was greatest with temozolomide and RT combined, followed by temozolomide, and then RT (FIG. 5). There was limited overlap between the top 100 genes that were positively and negatively correlated with response to RT, TMZ, and RT+TMZ (FIG. 6, Table S2). Within the top 10 pathways correlated with resistance to RT, GSEA revealed that several were related to the epithelial-mesenchymal transition and extracellular matrix interactions (FIG. 7A). RAS signaling pathways were represented in the top pathways correlated with TMZ resistance (FIG. 7B). For pathways correlated with resistance to RT+TMZ, 9 out of the top 10 pathways were associated with DNA replication (FIG. 7C).

TCGA

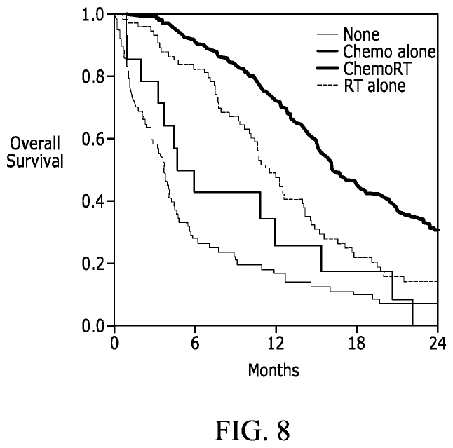

TCGA GBM (N=502) was utilized as the clinical validation cohort. Patients in this cohort were treated with combined ChemoRT (65%), RT alone (16%), chemotherapy alone (3%), or received no treatment (16%). Patients treated with ChemoRT had the best outcomes, followed by single modality treatment (RT or chemotherapy) and patients receiving no treatment had the worst outcomes (FIG. 8). MGMT promoter methylation was highly inversely correlated with MGMT gene expression (Spearman's rho=−0.54, p<0.0001), as expected, since promoter methylation silences MGMT, and is consistent with the literature²⁵.

Alkylating Chemotherapy Response Signature

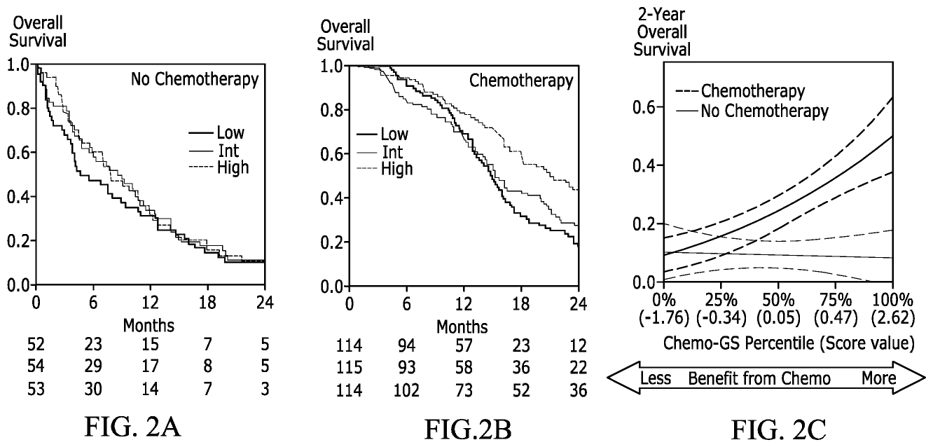

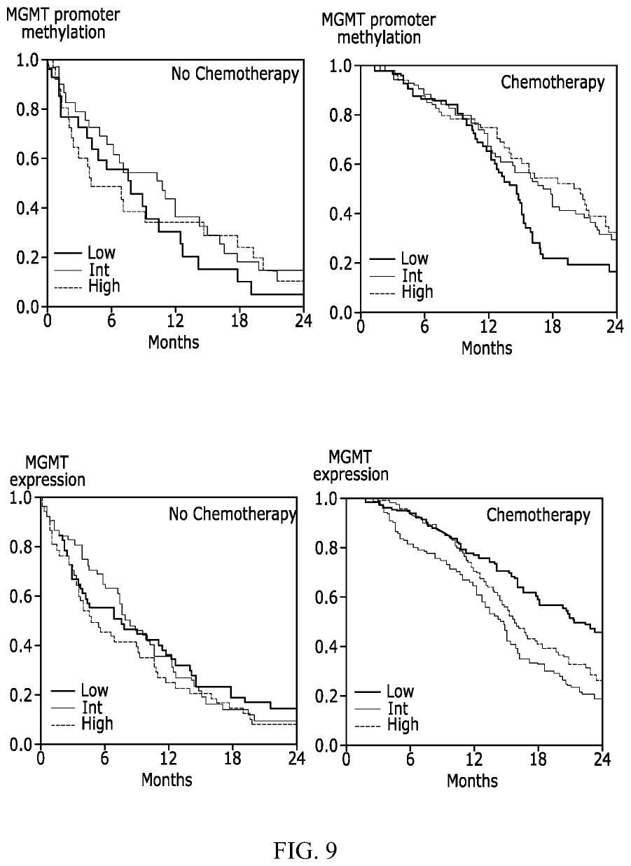

We ranked genes for correlation to TMZ response in the PDX models and found that MGMT had the second-highest ranked absolute correlation coefficient (Spearman's rho: −0.47). Because of the known biology of MGMT promoter methylation and increased sensitivity to alkylating chemotherapy²⁶, we reasoned that this finding served as a biologic positive control supporting our methodology and should be included in any genomic signature. Therefore, we hypothesized that a gene signature consisting of the average of MGMT and the only gene ranked higher (GPRASP1, Spearman's rho=−0.48) would predict response to chemotherapy. The absolute correlation of the two-gene Chemo-GS was higher than MGMT alone (Spearman's rho: −0.53) supporting the addition of GPRASP1. To validate in TCGA, we compared patients who received chemotherapy (with or without RT) to patients who did not (RT alone or no treatment). The Chemo-GS was associated with improved survival only in patients that received chemotherapy (p<0.0001, HR=0.66 [0.55-0.8]), but not in those that did not (p=0.14, HR=0.81 [0.62-1.07]; FIGS. 2A-2B). Higher Chemo-GS indicated an increased benefit from chemotherapy (FIG. 2C). MGMT promoter methylation was borderline associated with survival in patients who received chemotherapy (p=0.065, HR=0.86 [0.74-1.01]) and not associated in patients who did not receive chemotherapy (p=0.96, HR=1.01 [0.78-1.30]; FIGS. 9A-9B). MGMT gene expression was borderline associated with survival in the chemotherapy treated patients (p=0.085, HR=1.1 [0.99-1.23]) and not associated in patients who did not receive chemotherapy (p=0.48, HR=1.07 [0.89-1.28]; FIGS. 9C-9D). The MVA interactions were not significant for Chemo-GS (Table 1), MGMT promoter methylation (p=0.64) or MGMT expression (p=0.25).

TABLE 1

| | Chemo-GS¹ | | | RT-GS² | | | ChemoRT-GS³ | | |
|---|---|---|---|---|---|---|---|---|---|
| | P-value | HR | | P-value | HR | | P-value | HR | |
| Chemo-GS:Chemo | 0.8934 | 0.98 | [0.72-1.33] | Not included | | | Not included | | |
| RT-GS:RT | Not included | | | 0.0009 | 0.4 | [0.23-0.69] | Not included | | |
| ChemoRT- | Not included | | | Not included | | | 0.0204 | 0.56 | [0.34-0.91] |
| GS:ChemoRT | | | | | | | | | |
| GS | 0.0284 | 0.76 | [0.6-0.97] | 0.0019 | 2.26 | [1.35-3.77] | 0.5467 | 1.13 | [0.76-1.68] |
| Chemo | 0.2085 | 0.68 | [0.37-1.24] | 0.0608 | 0.56 | [0.3-1.03] | 0.1876 | 0.67 | [0.36-1.22] |
| RT | <0.0001 | 0.41 | [0.29-0.57] | <0.0001 | 0.36 | [0.26-0.51] | <0.0001 | 0.43 | [0.3-0.6] |
| ChemoRT | 0.8923 | 0.96 | [0.49-1.85] | 0.7482 | 1.12 | [0.57-2.18] | 0.8708 | 0.95 | [0.49-1.84] |
| Prior treatment | 0.1815 | 0.71 | [0.42-1.18] | 0.3877 | 0.8 | [0.48-1.33] | 0.2474 | 0.74 | [0.45-1.23] |
| Resection | 0.2021 | 1.21 | [0.9-1.63] | 0.3255 | 1.16 | [0.87-1.55] | 0.2664 | 1.18 | [0.88-1.58] |
| Male vs. Female | 0.2974 | 1.12 | [0.91-1.37] | 0.2673 | 1.12 | [0.91-1.38] | 0.2486 | 1.13 | [0.92-1.4] |
| Age | <0.0001 | 1.03 | [1.02-1.04] | <0.0001 | 1.03 | [1.02-1.04] | <0.0001 | 1.03 | [1.02-1.04] |

¹Left: interaction MVA for the Chemo-GS (as a continuous variable) with alkylating chemotherapy.
²Middle: interaction MVA for the RT-GS (as a continuous variable) with RT.
³Right: interaction MVA for the ChemoRT-GS (as a continuous variable) with alkylating chemotherapy and RT.

Radiation Response Signature

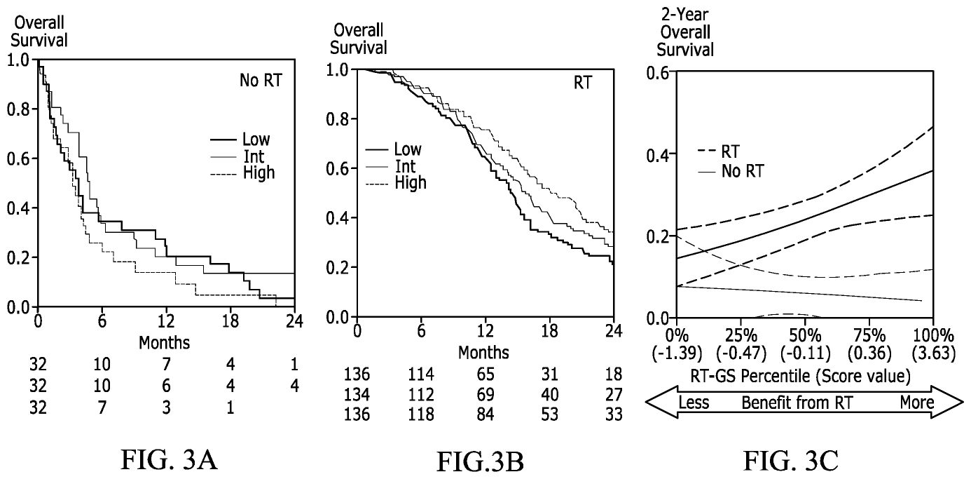

We next examined RT response, for which there are no clinically utilized predictive markers in GBM. Applying the exact same methodology used to generate Chemo-GS, we integrated the top two most correlated genes from the PDXs into RT-GS (average of CHGA, MAPK8, Spearman's rho=0.47, 0.41 respectively). In TCGA, we compared patients who received RT (with or without chemotherapy) to those that did not (chemotherapy alone or no treatment). The two-gene RT-GS was associated with improved survival only in the patients who received RT (p=0.0031, HR=0.78 [0.66-0.92]) and not in patients who did not receive RT (p=0.28, HR=1.28 [0.82-2.0]; FIGS. 3A-3B). Higher RT-GS scores indicated more of a benefit from RT (FIG. 3C). On interaction MVA, the RT-GS:RT treatment interaction term was highly significant (p=0.0009, Table 1) indicating that RT-GS is a predictive biomarker for response to radiation.

Chemotherapy and Radiation Response Signature

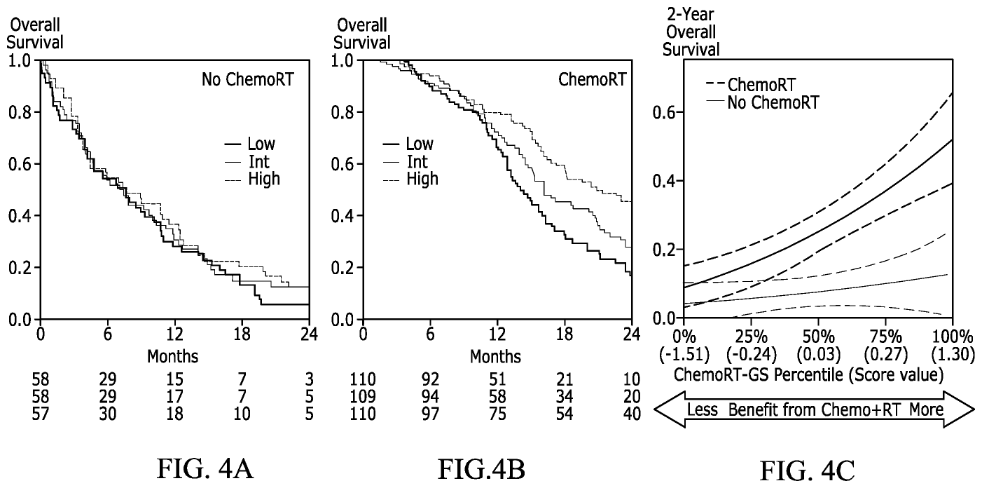

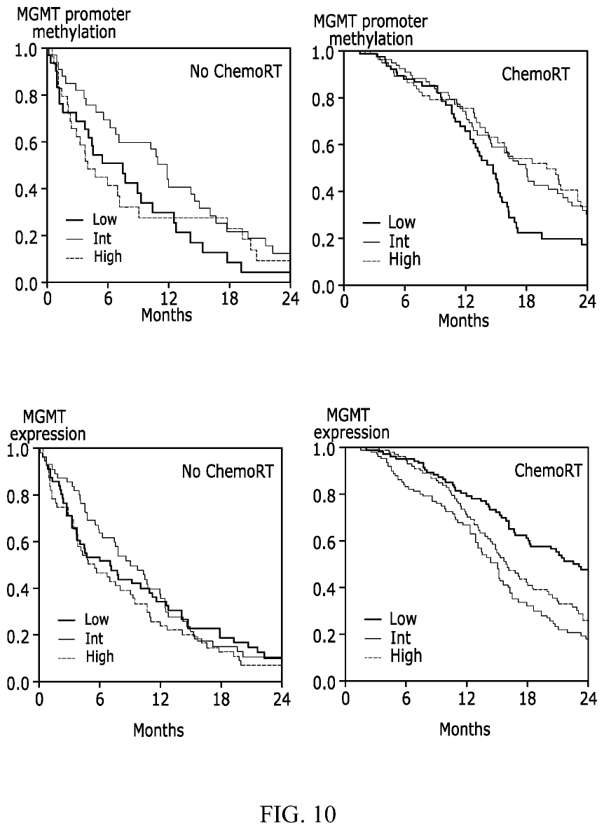

We next examined response to combined modality therapy, for which there is also no clinically utilized predictive marker. Since chemotherapy and RT response may independently contribute to ChemoRT response, we utilized the chemotherapy and RT response signatures from above, as well as the top two genes specifically correlated with RT+TMZ treatment in the PDXs (ATP6V0A2, FGF7, Spearman's rho=−0.7, −0.69 respectively) to develop a six-gene ChemoRT-GS. We then compared patients who received ChemoRT with those who had received single modality treatment or no treatment. As with the other two signatures, ChemoRT-GS was associated with improved survival only in patients treated with ChemoRT (p=0.0001, HR=0.54 [0.4-0.74]) and not in those not treated with ChemoRT (p=0.26, HR=0.8 [0.54-1.18]; FIG. 4A-C). The multivariate interaction term was significant (p=0.02, Table 1) indicating that ChemoRT-GS is a predictive biomarker for response to dual therapy with ChemoRT. MGMT promoter methylation was associated with survival in patients who received ChemoRT (p=0.033, HR=0.84 [0.71-0.99]) and not associated in patients who did not receive ChemoRT (p=0.79, HR=1.03 [0.82-1.31]; FIGS. 10A-10B), with a non-significant MVA interaction (p=0.55). Similarly, MGMT gene expression was borderline associated with survival in patients who received ChemoRT (p=0.057, HR=1.11 [1.00-0.25]) and not associated in patients who did not receive ChemoRT (p=0.45, HR=1.07 [0.9-1.27]; FIGS. 10C-10D), with a non-significant MVA interaction (p=0.77).

Clinical and Molecular Associations

Associations between the three signatures and clinical and molecular variables are presented in Tables S3-5. Of note, Chemo-GS was associated with MGMT promoter methylation as expected since MGMT gene expression is part of the signature. Chemo-GS was also associated with age at diagnosis. Higher RT-GS scores were also associated with younger age at diagnosis, consistent with the observation that younger patients may benefit more from RT²⁷. Similarly, ChemoRT-GS was associated with both MGMT promoter methylation and age, which is expected as both Chemo-GS and RT-GS are components of ChemoRT-GS. Scores of all three signatures were higher in IDH1-mutant tumors, suggesting that patients whose tumors harbor the IDH1 mutation may derive increased benefit from multiple therapies. When we include IDH1 mutation as a covariate in the MVA interaction analysis, the signature:treatment interactions remain significant for both RT-GS (p=0.025) and ChemoRT-GS (p=0.042), suggesting that the IDH1 mutation is not exclusively responsible for the predictive nature of these signatures.

### DISCUSSION

In the first study of its kind, we have successfully utilized a PDX-based approach to develop three different gene signatures to predict GBM responsiveness to chemotherapy, radiation and the combination. We independently validated these signatures in a clinical cohort of GBM patients. Each signature was prognostic only in patients receiving the signature-associated treatment. RT-GS and ChemoRT-GS represent the first molecular predictors of RT and ChemoRT response in GBM. The significant interaction between signatures and treatments indicate that they predict response to therapy rather than simply being prognostic.

The pathways associated with treatment resistance are consistent with known biology. MGMT, which predicts for temozolomide (TMZ) resistance in patients and laboratory models of GBM²⁶, was the second most highly correlated gene with TMZ resistance in our PDX model system. GSEA also revealed biologically relevant pathways associated with therapy resistance. Pathways involved with epithelial-to-mesenchymal transition were associated with GBM PDX radioresistance. This finding is in agreement with literature reports in GBM and other cancers and suggests that therapeutic approaches targeting this phenotype should be explored in combination with radiotherapy in GBM²⁸⁻³⁰. Increased expression of RAS signaling pathways was associated with TMZ resistance, which could be due to the role of RAS/MAPK signaling in cell survival³¹. Numerous pathways related to DNA elongation and replication were associated with resistance to combined TMZ and radiation treatment, perhaps indicating that this machinery allows GBMs to detoxify the complex DNA damage that forms when radiation is combined with alkylating chemotherapy³².

There is clinical utility of these gene signatures. Patients with a high ChemoRT-GS score should strongly consider standard combination therapy, whereas patients with a low score could be offered trials with novel therapy strategies. Patients with high RT-GS scores but low RT-Chemo and ChemoRT-GS scores may be excellent candidates for trials involving standard radiation but novel systemic therapy and/or novel radiosensitizing strategies. In patients only able to tolerate single modality treatment, the RT-GS and Chemo-GS scores could be used to select RT or TMZ with more precision than the currently-used MGMT promoter methylation assay⁵⁶.

In this study, PDX RNAseq data was obtained at a single time point, while treatment response experiments were performed numerous times over several years, during which mouse-specific evolution could have occured³³. It is contemplated that for future work PDX RNAseq data are obtained at multiple time points. Despite dertain limitations of this study, MGMT was the second-most correlated gene with temozolomide response, which underscores the validity of this model. Further work with additional cohorts, preferably with randomized trials, would increase the validity of the gene signature. Fortunately, validation is simplified by the platform-independent nature of these gene signatures, which were developed on RNAseq but validated on microarrays.

This xenograft-driven approach is versatile and generated biomarkers of response for three distinct treatments. Ideally, predictive biomarkers could be developed in randomized trials, but given the expense of both running the trial and profiling the tumors, PDXs may be a more feasible alternative for hypothesis generation. Another advantage is that placebo-treated but genomically identical PDXs serve as their own controls, which allows for cleaner comparisons of gene-level effects compared to genomically heterogenous clinical controls. High-throughput drug screening is possible using xenograft platforms' which could allow for potential biomarkers of response to be developed in-vivo prior to clinical trials. This would allow for initial trials to be biomarker-selected and potentially improve response rates for therapies that may only work in subset of patients.

As oncology moves towards the molecular classification of tumors, there is a strong need for molecular signatures that not only risk-stratify patients (prognostic biomarkers) but can also guide treatment decisions (predictive biomarkers). The gene signatures presented herein represent a promising initial step. If these signatures are validated in additional datasets, they could be used in the next generation of biomarker-stratified clinical trials and bring us closer to being able to truly personalize therapies for patients with GBM.

### Example 3

This example describes the supplemental methods carried out.

Patient Derived Xenografts

Between one and 15 experiments were performed per xenograft. The radiation dose was 20Gy in 2Gy/fraction either daily or BID in almost all of experiments. The TMZ doses were more varied. However, since no relationship was seen between TMZ dose and survival benefit in either the TMZ alone or TMZ+RT cohorts, all dose levels were included and treated equally. Full treatment data and PDX experimental results will be made available online at the Mayo Clinic PDX National Resource website: www.mayo.edu/research/labs/translational-neuro-oncology/mayo-clinic-brain-tumor-patient-derived-xenograft-national-resource

RNAseq library preparation was performed using the Illumina TruSeq RNA Sample Prep Kit V2. Mouse RNAseq reads were filtered out using Xenome 1.0.1 (Conway et al., Xenome—a tool for classifying reads from xenograft samples. Bioinformatics 2012; 28:i172-8), and mouse-only reads were excluded. RNAseq data were processed using a comprehensive bioinformatics pipeline from the Mayo Clinic: Map-RSeq (Kalari et al., BMC Bioinformatics 2014; 15:224). Gene expression was quantified using RPKM. The full dataset is in the process of being deposited in cBioPortal (http://www.cbioportal.org).

TCGA

Available clinical and molecular variables such as age, sex, treatment, prior treatment, surgery, IDH1, and MGMT were used. However, not all clinical variables had complete and well annotated data (e.g., performance status). MGMT promotor methylation was assessed using the same method as in TCGA utilizing the Illumina Infinium HumanMethylation27 and 450 BeadChip arrays (Bady et al., Acta Neruopathol 2012; 124:547-60; Brennan et al., Cell 2013; 155: 462-77). In the 5 samples where both 27 k and 450 k data were available for the same probes, the values were averaged. MGMT promoter methylation was scaled by the standard deviation to make hazard ratios comparable with gene expression and the signatures.

Gene Signature Development

Some PDXs had treatment responses assessed in multiple experiments and we treated each experiment, rather than each PDX, as an individual data point. Only genes that were available in both the PDX and TCGA samples were retained for analysis. Genes were ranked by the absolute value of the correlation coefficient in order to identify potential biomarkers for predicting treatment response. We did not utilize a more complex statistical model due to the significant technical differences between RNAseq (PDXs) and microarrays (TCGA) to prevent overfitting. Furthermore, since the correlation coefficients in the top genes were very similar, genes were weighted equally by utilizing a simple average. Genes with negative correlations were multiplied by −1 to invert their signs. Thus, increasing values are associated with increased treatment sensitivity, and decreasing values are associated with treatment resistance, allowing for combining of genes that were positively and negatively correlated to treatment resistance.

The formulas for the three gene signatures are below (assuming centered and scaled gene expression data):

Chemo-GS=(−GPRASP1+−MGMT)/2

RT-GS=(CHGA+MAPK8)/2

ChemoRT-GS=(−GPRASP1+−MGMT+CHGA+MAPK8+−ATP6V0A2+−FGF7)/6

Gene Set Enrichment Analysis

The ranked gene list by Spearman's correlation coefficient from above was also utilized to run GSEA pre-ranked. The default parameters were used, and the following gene sets were included for assessment: H1:Hallmarks, C2:Canonical Pathways, and C5:GO Biological

Processes. Volcano plots were created by plotting the normalized enrichment scores versus the −log of the p-values from GSEA.

### Example 4

This example describes an exemplary method of the present disclosure.

A sample is obtained from a human subject diagnosed with glioblastoma by biopsy or surgical resection. Total RNA is isolated from the sample using a commercial RNA extraction kit (e.g. AllPrep DNA/RNA mini kit).

Gene expression microarrays are then used to quantify the expression of the RNA isolated from the sample. Briefly, the RNA is first reverse transcribed, amplified, fragmented and labeled and then hybridized to a microarray (e.g. Affymetrix Human U133A microarray).

The expression levels for the six genes MGMT, GPRASP1, CHGA, MAPK8, ATP6V0A2 and FGF7 is centered and scaled using software such as the R scale function. A score is then calculated (using the centered and scaled expression level for each gene) using Equations 1-3, wherein Equation 1 is for determining a Chemo-GS score, Equation 2 is for determining a RT-GS score and Equation 3 is for determining a ChemoRT-GS score:

| | |
|---|---|
| Equation 1 | (−GPRASP1 + −MGMT)/2 |
| Equation 2 | (CHGA + MAPK8)/2 |
| Equation 3 | (−GPRASP1 + −MGMT + CHGA + |
| | MAPK8 + −ATP6V0A2 + −FGF7)/6 |

Each score is used to determine the % of 2-year overall survival upon treatment with and without alkylating chemotherapy, radiation therapy, or the combination of alkylating chemotherapy and radiation therapy using the graphs of FIGS. 2C, 3C, and 4C. For example, if Equation 1 yielded a Chemo-GS score of 0.1, Equation 2 yielded a RT-GS score of 0.72, and Equation 3 yielded a ChemoRT-GS score of 7, the % chance for a 2-year overall survival, according to FIGS. 2C, 3C, and 4C, are 50% (FIG. 2C), a 75% (FIG. 3C) and ^{˜}90% (FIG. 4C) compared to ^{˜}10% without treatment. The recommended treatment for the subject is the combination of alkylating chemotherapy and radiation therapy, because this treatment yielded the highest improvement in % chance of 2-year overall survival (^{˜}90% vs. 50% for alkylating chemotherapy alone, 75% for radiation therapy alone, ^{˜}10% with no treatment.

### Example 5

This example describes an exemplary method of the present disclosure.

In Example 4, the graphs of FIGS. 2C, 3C, and 4C were created based on population data from patients diagnosed with glioblastoma from the TCGA cohort, as essentially described in Examples 1-3. Similar graphs can be made using expression data from patients diagnosed with glioblastoma of different cohorts. Briefly, expression data for the six genes (MGMT, GPRASP1, CHGA, MAPK8, ATP6V0A2 and FGF7) from the Michigan Brain Tumor Bank can be analyzed as essentially described in Examples 1-3. In exemplary alternative or additional instances, expression data for the six genes (MGMT, GPRASP1, CHGA, MAPK8, ATP6V0A2 and FGF7) of a population of patients diagnosed with glioblastoma who have not yet received any glioblastoma treatment is analyzed as essentially described in Examples 1-3. Validation of the particular genes for the chemotherapy, radiation and combination gene signatures is accomplished in this manner.

Using the data from these additional cohorts/tissue banks, graphs similar to those in FIGS. 2C, 3C, and 4C are made and used to determine the % of 2-year overall survival upon treatment with alkylating chemotherapy, radiation therapy, or the combination of alkylating chemotherapy and radiation therapy. Selection of the best treatment modality for a given patient may be made based on these graphs.

### Example 6

This example describes an alternative methodology using a known and commonly used regression model.

RNA-Seq was conducted to quantify the expression of the RNA isolated from the sample. Briefly, RNAseq library preparation was performed using the Illumina TruSeq RNA Sample Prep Kit V2. Mouse RNAseq reads were filtered out using Xenome 1.0.11, and mouse-only reads were excluded. RNAseq data were processed using a comprehensive bioinformatics pipeline from the Mayo Clinic: Map-RSeq.

The expression levels for the top 10, top 25, top 50, and top 100 genes most correlated to radiation, chemotherapy, and chemoRT response in patient derived xenografts were processed through an elastic net regression model as essentially described in Zhao et al., Lancet Oncology (2016) Volume 17, No. 11, p1612-1620, November 2016, using the R glmnet package, instead of using the formulas as essentially described above in Examples 1-4. This approach relies on the glmnet algorithm to select the important genes and weight them appropriately.

In contrast to using the six genes in the formulas described above, it was observed that using glmnet on a wide range of genes resulted in models which were not statistically significant only in the treated patients, whereas, as described above in Examples 1-3, centering and scaling the expression levels of the six genes and calculating scores as above resulted in models which were statistically significant only in the patients receiving radiation, chemotherapy, or both (p<0.05).

### REFERENCES

The following references are cited throughout according to the number below.
1. Byron et al., Clin Cancer Res 2018; 24(2): 295-305.
2. Colman et al., Neuro Oncol 2010; 12(1): 49-57.
3. Hegi et al., N Engl J Med 2005; 352(10): 997-1003.
4. Perry et al., N Engl J Med 2017; 376(11): 1027-37.
5. Wick et al., Lancet Oncol 2012; 13(7): 707-15.
6. Malmstrom et al., Lancet Oncol 2012; 13(9): 916-26.
7. Combs et al., Radiat Oncol 2011; 6: 115.
8. Ahmed et al., Oncotarget 2015; 6(33): 34414-22.
9. Speers et al., Clin Cancer Res 2015; 21(16): 3667-77.
10. Lee et al., Oncotarget 2015; 6(28): 25619-30.
11. Stewart et al., Journal of clinical oncology: official journal of the American Society of Clinical Oncology 2015; 33(22): 2472-80.
12. Rubio-Viqueira et al., Clin Cancer Res 2006; 12(15): 4652-61.
13. Bertotti et al., Cancer Discov 2011; 1(6): 508-23.
14. Gao et al., Nat Med 2015; 21(11): 1318-25.
15. Carlson et al., Intl Radiat Oncol Biol Phys 2009; 75(1): 212-9.
16. Kitange et al., Cell Rep 2016; 14(11): 2587-98.
17. Ma et al., Clin Cancer Res 2008; 14(9): 2601-8.
18. Brennan et al., Cell 2013; 155(2): 462-77.
19. Goldman et al., Nucleic Acids Res 2015; 43 (Database issue): D812-7.
20. Yau et al., Breast Cancer Res 2013; 15 (5): R103.
21. Tutt et al., BMC Cancer 2008; 8: 339.
22. Subramanian et al., Proc Natl Acad Sci USA 2005; 102(43): 15545-50.
23. Janes et al., Ann Intern Med 2011; 154(4): 253-9.
24. White et al., Eur Urol 2017; 71(2): 257-66.
25. Everhard et al., Neuro Oncol 2009; 11(4): 348-56.
26. Weller et al., Nat Rev Neurol 2010; 6(1): 39-51.
27. Barker et al., Neurosurgery 2001; 49(6): 1288-97; discussion 97-8.
28. Bhat Krishna et al., Cancer Cell 2013; 24(3): 331-46.
29. Chang et al., Cell Death & Amp; Disease 2013; 4: e875.
30. Davis et al., Trends in Pharmacological Sciences; 35(9): 479-88.
31. Bonni et al., Science 1999; 286(5443): 1358-62.
32. Morgan et al., Cancer discovery 2014; 4(3): 280-91.
33. Ben-David et al., Nature genetics 2017; 49(11): 1567-75.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A treatment regimen for use in a method of treating a tumor in a subject from whom a sample comprising a cell or cells from the tumor was obtained and the expression level of MGMT, GPRASP1, CHGA, MAPK8, ATP6V0A2, FGF7, or any combination thereof, of the sample was measured, wherein the treatment regimen comprises (i) an alkylating chemotherapy, (ii) radiation therapy, or (iii) a combination of alkylating chemotherapy and radiation therapy.

2. The treatment regimen of claim 1, wherein the expression levels of (A) MGMT and GPRASP1 or (B) CHGA and MAPK8, of the sample were measured.

3. The treatment regimen of claim 2, which is (A) an alkylating chemotherapy, when the measured expression levels of MGMT and GPRASP1 in the sample were decreased relative to a reference level, or (B) radiation therapy, when the measured expression levels of CHGA and MAPK8 in the sample were increased relative to a reference level.

4. The treatment regimen of any one of claims 1 to 3, wherein the expression levels of MGMT, GPRASP1, CHGA, MAPK8, ATP6V0A2, and FGF7 were measured.

5. The treatment regimen of claim 4, which is a combination comprising alkylating chemotherapy and radiation therapy, when the measured expression levels of MGMT, GPRASP1, ATP6V0A2, and FGF7 in the sample were decreased, relative to a reference level, and/or the measured expression levels of CHGA and MAPK8 in the sample were increased, relative to a reference level.

6. A treatment regimen for use in a method of treating a tumor in a subject having a decreased expression level of MGMT or GPRASP1, or both, relative to a reference level, said treatment regimen comprising an alkylating chemotherapy in an amount effective to treat the tumor.

7. A treatment regimen for use in a method of treating a tumor in a subject having an increased expression level of CHGA or MAPK8, or both, relative to a reference level, said treatment regimen comprising a radiation therapy in an amount effective to treat the tumor.

8. A treatment regimen for use in a method of treating a tumor in a subject having (A) a decreased expression level of MGMT, GPRASP1, ATP6V0A2, FGF7, or any combination thereof, relative to a reference level, or (B) an increased expression level of CHGA or MAPK8, or both, relative to a reference level, or (C) both (A) and (B), said treatment regimen comprising a combination comprising an alkylating chemotherapy and a radiation therapy in an amount effective to treat the tumor.

9. A treatment regimen for use in a method of treating a tumor in a subject, said method, comprising
a) measuring the expression level of MGMT, GPRASP1, CHGA, MAPK8, ATP6V0A2, FGF7, or any combination thereof, in a sample comprising a cell or cells from the tumor, and
b) administering to the subject the treatment regimen in an amount effective to treat the tumor, wherein the treatment regimen comprises (i) an alkylating chemotherapy, (ii) radiation therapy, or (iii) a combination of alkylating chemotherapy and radiation therapy, based on the expression level(s) measured in (a).

10. The treatment regimen of claim 9, wherein the method comprises measuring the expression levels of (A) MGMT and GPRASP1 or (B) CHGA and MAPK8.

11. The treatment regimen of claim 10, wherein the method comprises administering to the subject:
(A) an alkylating chemotherapy, when the measured expression levels of MGMT and GPRASP1 in the sample are decreased relative to a reference level, or
(B) radiation therapy, when the measured expression levels of CHGA and MAPK8 in the sample are increased relative to a reference level.

12. The treatment regimen of any one of claims 9 to 11, wherein the method comprises measuring the expression levels of MGMT, GPRASP1, CHGA, MAPK8, ATP6V0A2, and FGF7.

13. The treatment regimen of claim 12, wherein the method comprises administering to the subject a combination comprising alkylating chemotherapy and radiation therapy, when the measured expression levels of MGMT, GPRASP1, ATP6V0A2, and FGF7 in the sample are decreased, relative to a reference level, and/or the measured expression levels of CHGA and MAPK8 in the sample are increased, relative to a reference level.

14. A method of identifying a tumor as treatable with an alkylating chemotherapy, comprising
a) measuring the level of expression of MGMT or GPRASP1, or both, in a sample comprising a cell or cells from the tumor, and
b) identifying the tumor as treatable with an alkylating chemotherapy, when the level of MGMT, GPRASP1, or both, in the sample is decreased relative to a reference level.

15. A method of identifying a tumor as treatable with a radiation therapy, comprising
a) measuring the level of expression of CHGA or MAPK8, or both, in a sample comprising a cell or cells from the tumor, and
b) identifying the tumor as treatable with radiation therapy, when the level of CHGA, MAPK8, or both, in the sample is increased relative to a reference level.

16. A method of identifying a tumor as treatable with a combination comprising alkylating chemotherapy and radiation therapy, comprising
a) measuring the level of expression of MGMT, GPRASP1, CHGA, MAPK8, ATP6V0A2, FGF7, or any combination thereof, in a sample comprising a cell or cells from the tumor, and
b) identifying the subject with a tumor treatable with the combination, when (A) the expression level of MGMT, GPRASP1, ATP6V0A2, or FGF7, or any combination thereof, in the sample is decreased relative to a reference level, (B) the expression level of CHGA or MAPK8, or both, in the sample is increased relative to a reference level, or (C) both (A) and (B).

17. A method of determining a treatment for a subject with a tumor, comprising:
a) measuring the level of expression of MGMT, GPRASP1, CHGA, MAPK8, ATP6V0A2, FGF7, or any combination thereof, in a sample comprising a cell or cells from the tumor, and
b) selecting for the subject a treatment comprising (i) an alkylating chemotherapy, (ii) radiation therapy, or (iii) a combination of alkylating chemotherapy and radiation therapy based on the expression level.

18. The method of claim 17, comprising measuring the expression levels of (A) MGMT and GPRASP1 or (B) CHGA and MAPK8.

19. The method of claim 18, comprising selecting for the subject a treatment comprising:
(A) an alkylating chemotherapy, when the measured expression levels of MGMT and GPRASP1 in the sample are decreased relative to a reference level, or
(B) radiation therapy, when the measured expression levels of CHGA and MAPK8 in the sample are increased relative to a reference level.

20. The method of any one of claims 9 to 11, comprising measuring the expression levels of MGMT, GPRASP1, CHGA, MAPK8, ATP6V0A2, and FGF7.

21. The method of claim 20, comprising selecting for the subject a treatment comprising a combination of alkylating chemotherapy and radiation therapy, when the measured expression levels of MGMT, GPRASP1, ATP6V0A2, and FGF7 in the sample are decreased, relative to a reference level, and/or the measured expression levels of CHGA and MAPK8 in the sample are increased, relative to a reference level.

22. A method of determining a treatment for a subject from whom a sample was obtained and the expression level of MGMT, GPRASP1, CHGA, MAPK8, ATP6V0A2, FGF7, or any combination thereof, of the sample was measured, said method comprising selecting a treatment comprising (i) an alkylating chemotherapy, (ii) radiation therapy, or (iii) a combination of alkylating chemotherapy and radiation therapy, based on the expression level.

23. The method of claim 22, wherein the expression levels of (A) MGMT and GPRASP1 or (B) CHGA and MAPK8 were measured.

24. The method of claim 23, comprising selecting for the subject:
(A) an alkylating chemotherapy, when the measured expression levels of MGMT and GPRASP1 in the sample were decreased relative to a reference level, or
(B) radiation therapy, when the measured expression levels of CHGA and MAPK8 in the sample were increased relative to a reference level.

25. The method of any one of claims 22 to 24, wherein the expression levels of MGMT, GPRASP1, CHGA, MAPK8, ATP6V0A2, and FGF7 were measured.

26. The method of claim 25, comprising selecting for the subject a combination of alkylating chemotherapy and radiation therapy, when the measured expression levels of MGMT, GPRASP1, ATP6V0A2, and FGF7 in the sample were decreased, relative to a reference level, and/or the measured expression levels of CHGA and MAPK8 in the sample were increased, relative to a reference level.

27. The treatment regimen or method of any one of the previous claims, wherein the reference level is the corresponding expression level of MGMT, GPRASP1, CHGA, MAPK8, ATP6V0A2, or FGF7 in a population untreated subjects.

28. The treatment regimen or method of any one of the previous claims, wherein the alkylating chemotherapy comprises temozolomide (TMZ), CCNU, BCNU, or any combination thereof.

29. The treatment regimen or method of any one of the previous claims, wherein the radiation therapy comprises external beam radiation.

30. The treatment regimen or method of any one of the previous claims, wherein the tumor is a glioblastoma.

31. The treatment regimen or method of any one of the previous claims, wherein the cell of the tumor comprises mutations in EGFR, PTEN, and p53.

32. The treatment regimen or method of any one of the previous claims, further comprising obtaining the sample from the subject.

33. The treatment regimen or method of claim 32, comprising obtaining the sample by biopsy or surgical resection.

34. The treatment regimen or method of any one of the previous claims, wherein measuring the expression level comprises isolating RNA from the sample and quantifying the RNA by RNA-Seq.

35. The treatment regimen or method of any one of claims 1 to 34, wherein measuring the expression level comprises isolating RNA from the sample, producing complementary DNA (cDNA) from the RNA, amplifying the cDNA and hybridizing the cDNA to a gene expression microarray.

36. The treatment regimen or method of any one of the previous claims, comprising centering and scaling each measured expression level.

37. The treatment regimen or method of claim 36, wherein the measured expression level is centered and scaled relative to a reference level.

38. The treatment regimen or method of claim 37, wherein the reference level is the corresponding expression level of MGMT, GPRASP1, CHGA, MAPK8, ATP6V0A2, or FGF7 in a population untreated subjects.

39. The treatment regimen or method of any one of claims 36 to 38, wherein the centering and scaling of each measured expression level comprises:
(A) determining the mean of the corresponding expression level of MGMT, GPRASP1, CHGA, MAPK8, ATP6V0A2, or FGF7 of a population of subjects,
(B) calculating the standard deviation of the corresponding expression level of MGMT, GPRASP1, CHGA, MAPK8, ATP6V0A2, or FGF7 of the population;
(C) subtracting the mean determined in step (A) from the measured expression level to obtain a mean-adjusted expression level, and
(D) dividing the mean-adjusted expression level calculated in step (C) by the standard deviation calculated in (B) to obtain a centered and scaled expression level.

40. The treatment regimen or method of claim 39, further comprising calculating
(A) an alkylating chemotherapy score using Equation 1:
$$\frac{( - 1 ) [ G P R A S P 1 ) + ( - 1 ) ( MGMT )}{2} \qquad ( Equation 1 )$$
(B) a radiation therapy score using Equation 2:
$$\frac{( CHGA ) + ( MAPK 8 )}{2} ; \qquad ( Equation 2 )$$
and/or
(C) a combination alkylating chemotherapy/radiation therapy score by using Equation 3:
$$\frac{\begin{matrix}( - 1 ) ( GPRASP 1 ) + ( - 1 ) ( MGMT ) + C H GA + \\ MAPK 8 + ( - 1 ) [ A T P 6 V 0 A 2 ) + ( - 1 ) ( FGF 7 )\end{matrix}}{6} \qquad ( Equation 3 )$$
wherein the centered and scaled expression level for MGMT, GPRASP1, CHGA, MAPK8, ATP6V0A2, and/or FGF7 is used.

41. The treatment regimen or method of claim 40, wherein an alkylating chemotherapy score, a radiation therapy score, and a combination alkylating chemotherapy/radiation therapy score is calculated.

42. The treatment regimen or method of claim 41, further comprising using each score to determine a percent chance of overall survival for treatment with alkylating chemotherapy.

43. The treatment regimen or method of claim 41, further comprising using each score to determine a percent chance of overall survival for treatment with radiation therapy.

44. The treatment regimen or method of claim 41, further comprising using each score to determine a percent chance of overall survival for treatment with a combination of alkylating chemotherapy and radiation therapy.

45. The treatment regimen or method of claim 42, further comprising calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination, and selecting the treatment with the greatest difference in percent chance of overall survival with the treatment vs. without the treatment.

46. A kit comprising at least two different nucleic acid probes, wherein the nucleic acid probes are specific for at least two of MGMT, GPRASP1, CHGA, MAPK8, ATP6V0A2, and FGF7.

47. The kit of claim 46, comprising (A) nucleic acid probes specific for MGMT and GPRASP1, (B) nucleic acid probes specific for CHGA and MAPK8, (C) nucleic acid probes specific for ATP6V0A2, and FGF7, or (D) a combination thereof.

48. The kit of claim 46, further comprising a component of a therapeutic regimen according to any one of claims 1-13, optionally, wherein the component is an alkylating chemotherapeutic agent.

49. Use of the kit of any one of claims 46-48 in a method of any one of the preceding claims.

50. A system comprising machine readable instructions that, when executed by the processor, cause the processor to:
(i) receive a measured expression level of a sample obtained from a subject with a tumor for each of MGMT, GPRASP1, CHGA, MAPK8, ATP6V0A2, or FGF7;
(ii) receive a plurality of data values, each data value is a measured expression level of MGMT, GPRASP1, CHGA, MAPK8, ATP6V0A2, or FGF7 among a population of subjects;
(iii) for each of MGMT, GPRASP1, CHGA, MAPK8, ATP6V0A2, and FGF7, calculate a mean and a standard deviation of the data values received in step (ii);
(iv) subtract the mean from the corresponding measured expression level to obtain a mean-adjusted expression level for each of MGMT, GPRASP1, CHGA, MAPK8, ATP6V0A2, and FGF7,
(v) divide each mean-adjusted expression level by the corresponding standard deviation to obtain a centered and scaled expression level;
(vi) calculate an alkylating chemotherapy score using Equation 1:
$$\frac{( - 1 ) [ G P R A S P 1 ) + ( - 1 ) ( MGMT )}{2} \qquad ( Equation 1 )$$
(B) a radiation therapy score using Equation 2:
$$\frac{( CHGA ) + ( MAPK 8 )}{2} ; \qquad ( Equation 2 )$$
and/or
(C) a combination alkylating chemotherapy/radiation therapy score by using Equation 3:
$$\frac{\begin{matrix}( - 1 ) ( GPRASP 1 ) + ( - 1 ) ( MGMT ) + C H GA + \\ MAPK 8 + ( - 1 ) [ A T P 6 V 0 A 2 ) + ( - 1 ) ( FGF 7 )\end{matrix}}{6} \qquad ( Equation 3 )$$
wherein “MGMT”, “GPRASP1”, “CHGA”, “MAPK8”, “ATP6V0A2”, and “FGF7” is the centered and scaled expression level as determined in (v);
(vii) use the alkylating chemotherapy score to determine a percent chance of overall survival for treatment with alkylating chemotherapy, the radiation therapy score to determine percent chance of overall survival for treatment with radiation therapy, and combination alkylating chemotherapy/radiation therapy score to determine percent chance of overall survival for treatment with a combination of alkylating chemotherapy and radiation therapy; and calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination, and
(viii) select the treatment with the greatest difference in percent chance of overall survival with the treatment vs. without the treatment.

51. The system of claim 50, wherein each subject of the population is a subject with a tumor which has not been treated for the tumor.

52. A computer-readable storage media having stored thereon machine-readable instructions executable by a processor, wherein the instructions comprise
(i) instructions for receiving a measured expression level of a sample obtained from a subject with a tumor for each of MGMT, GPRASP1, CHGA, MAPK8, ATP6V0A2, or FGF7;
(ii) instructions for receiving a plurality of data values, each data value is a measured expression level of MGMT, GPRASP1, CHGA, MAPK8, ATP6V0A2, or FGF7 among a population of subjects;
(iii) for each of MGMT, GPRASP1, CHGA, MAPK8, ATP6V0A2, and FGF7, instructions for calculating a mean and a standard deviation of the data values received in step (ii);
(iv) instructions for subtracting the mean from the corresponding measured expression level to obtain a mean-adjusted expression level for each of MGMT, GPRASP1, CHGA, MAPK8, ATP6V0A2, and FGF7,
(v) instructions for dividing each mean-adjusted expression level by the corresponding standard deviation to obtain a centered and scaled expression level;
(vi) instructions for calculating an alkylating chemotherapy score using Equation 1:
$$\frac{( - 1 ) [ G P R A S P 1 ) + ( - 1 ) ( MGMT )}{2} \qquad ( Equation 1 )$$
(B) a radiation therapy score using Equation 2:
$$\frac{( CHGA ) + ( MAPK 8 )}{2} ; \qquad ( Equation 2 )$$
and/or
(C) a combination alkylating chemotherapy/radiation therapy score by using Equation 3:
$$\frac{\begin{matrix}( - 1 ) ( GPRASP 1 ) + ( - 1 ) ( MGMT ) + C H GA + \\ MAPK 8 + ( - 1 ) [ A T P 6 V 0 A 2 ) + ( - 1 ) ( FGF 7 )\end{matrix}}{6} \qquad ( Equation 3 )$$
wherein “MGMT”, “GPRASP1”, “CHGA”, “MAPK8”, “ATP6V0A2”, and “FGF7” is the centered and scaled expression level as determined in (v);
(vii) instructions for using the alkylating chemotherapy score to determine a percent chance of overall survival for treatment with alkylating chemotherapy, the radiation therapy score to determine percent chance of overall survival for treatment with radiation therapy, and combination alkylating chemotherapy/radiation therapy score to determine percent chance of overall survival for treatment with a combination of alkylating chemotherapy and radiation therapy; calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination, and
(viii) instructions for selecting the treatment with the greatest difference in percent chance of overall survival with the treatment vs. without the treatment.

53. The computer-readable storage media of claim 52, wherein each subject of the population is a subject with a tumor which has not been treated for the tumor.

54. A method implemented by a processor in a computer, the method comprising the steps of:
(i) receiving a measured expression level of a sample obtained from a subject with a tumor for each of MGMT, GPRASP1, CHGA, MAPK8, ATP6V0A2, or FGF7;
(ii) receiving a plurality of data values, each data value is a measured expression level of MGMT, GPRASP1, CHGA, MAPK8, ATP6V0A2, or FGF7 among a population of subjects;
(iii) for each of MGMT, GPRASP1, CHGA, MAPK8, ATP6V0A2, and FGF7, calculating a mean and a standard deviation of the data values received in step (ii);
(iv) subtracting the mean from the corresponding measured expression level to obtain a mean-adjusted expression level for each of MGMT, GPRASP1, CHGA, MAPK8, ATP6V0A2, and FGF7,
(v) dividing each mean-adjusted expression level by the corresponding standard deviation to obtain a centered and scaled expression level;
(vi) calculating an alkylating chemotherapy score using Equation 1:
$$\frac{( - 1 ) [ G P R A S P 1 ) + ( - 1 ) ( MGMT )}{2} \qquad ( Equation 1 )$$
(B) a radiation therapy score using Equation 2:
$$\frac{( CHGA ) + ( MAPK 8 )}{2} ; \qquad ( Equation 2 )$$
and/or
(C) a combination alkylating chemotherapy/radiation therapy score by using Equation 3:
$$\frac{\begin{matrix}( - 1 ) ( GPRASP 1 ) + ( - 1 ) ( MGMT ) + C H GA + \\ MAPK 8 + ( - 1 ) [ A T P 6 V 0 A 2 ) + ( - 1 ) ( FGF 7 )\end{matrix}}{6} \qquad ( Equation 3 )$$
wherein “MGMT”, “GPRASP1”, “CHGA”, “MAPK8”, “ATP6V0A2”, and “FGF7” is the centered and scaled expression level as determined in (v);
(vii) using the alkylating chemotherapy score to determine a percent chance of overall survival for treatment with alkylating chemotherapy, the radiation therapy score to determine percent chance of overall survival for treatment with radiation therapy, and combination alkylating chemotherapy/radiation therapy score to determine percent chance of overall survival for treatment with a combination of alkylating chemotherapy and radiation therapy; and calculating the difference between the percent chance of overall survival for treatment with alkylating chemotherapy to the percent chance of overall survival for treatment without alkylating chemotherapy, the difference between the percent chance of overall survival for treatment with radiation therapy to the percent chance of overall survival for treatment without radiation therapy, and the difference between the percent chance of overall survival for treatment with a combination of alkylating chemotherapy and radiation therapy to the percent chance of overall survival for treatment without the combination, and
(viii) selecting the treatment with the greatest difference in percent chance of overall survival with the treatment vs. without the treatment.


SEQUENCE LISTING (30 sequences)

SEQ ID NO: 1 - DNA, 896 bp, Homo sapiens
  misc_feature: MGMT Isoform X1
acttggtgagtgtctgggtcgcctcgctcccggaagagtgcggagctctccctcgggacggtggcagcctcgagtggtcctgcaggcgccctcacttcgccgtcgggtgtggggccgccctgacccccacccatcccgggcgagctccaggtacttggaaaaatggacaaggattgtgaaatgaaacgcaccacactggacagccctttggggaagctggagctgtctggttgtgagcagggtctgcacgaaataaagctcctgggcaaggggacgtctgcagctgatgccgtggaggtcccagcccccgctgcggttctcggaggtccggagcccctgatgcagtgcacagcctggctgaatgcctatttccaccagcccgaggctatcgaagagttccccgtgccggctcttcaccatcccgttttccagcaagagtcgttcaccagacaggtgttatggaagctgctgaaggttgtgaaattcggagaagtgatttcttaccagcaattagcagccctggcaggcaaccccaaagccgcgcgagcagtgggaggagcaatgagaggcaatcctgtccccatcctcatcccgtgccacagagtggtctgcagcagcggagccgtgggcaactactccggaggactggccgtgaaggaatggcttctggcccatgaaggccaccggttggggaagccaggcttgggagggagctcaggtctggcaggggcctggctcaagggagcgggagctacctcgggctccccgcctgctggccgaaactgagtatgtgcagtaggatggatgtttgagcgacacacacgtgtaacactgcatcggatgcggggcgtggaggcaccgctgtattaaaggaagtggcagtgtcctgggaacaa

SEQ ID NO: 2 - protein, 207 aa, Homo sapiens
  MISC_FEATURE: MGMT Isoform X1
MDKDCEMKRTTLDSPLGKLELSGCEQGLHEIKLLGKGTSAADAVEVPAPAAVLGGPEPLMQCTAWLNAYFHQPEAIEEFPVPALHHPVFQQESFTRQVLWKLLKVVKFGEVISYQQLAALAGNPKAARAVGGAMRGNPVPILIPCHRVVCSSGAVGNYSGGLAVKEWLLAHEGHRLGKPGLGGSSGLAGAWLKGAGATSGSPPAGRN

SEQ ID NO: 3 - DNA, 4558 bp, Homo sapiens
  misc_feature: MGMT Isoform X1
ggaatggtagagaacagtggagaagtagatgcttgtcaggcgtcttgtctgctgggccaaggtgaatcaggagtgccgtggaggtcccagcccccgctgcggttctcggaggtccggagcccctgatgcagtgcacagcctggctgaatgcctatttccaccagcccgaggctatcgaagagttccccgtgccggctcttcaccatcccgttttccagcaagagtcgttcaccagacaggtgttatggaagctgctgaaggttgtgaaattcggagaagtgatttcttaccagcaattagcagccctggcaggcaaccccaaagccgcgcgagcagtgggaggagcaatgagaggcaatcctgtccccatcctcatcccgtgccacagagtggtctgcagcagcggagccgtgggcaactactccggaggactggccgtgaaggaatggcttctggcccatgaaggccaccggttggggaagccaggcttgggagggagctcaggtctggcaggggcctggctcaagggagcgggagctacctcgggctccccgcctgctggccgaaactgagtatgtgcagtaggatggatgtttgagcgacacacacgtgtaacactgcatcggatgcggggcgtggaggcaccgctgtattaaaggaagtggcagtgtcctgggaacaagcgtgtctgccctttctgtttccatattttacagcaggatgagttcagacgcccgcggtcctgcacacatttgtttccttctctaacgctgcccttgctctatttttcatgtccattaaaacaggccaagtgagtgtggaaggcctggctcatgttgggccacagcccaggatggggcagtctggcaccctcaggccacagacggctgccatagccgctgtccagggccagctaaggcccatcccaggccgtccacactagaaagctggccctgccccatccccaccatgcctcccttcctggctgtgtccatggctgtgatggcattctccactcagcagttcctagcatcccacacccaggtctcactgaaagaaaggggaacaggccatggcagtcagtgcttacagaggttttctagctttgtttcctacctgccaaaaaggctaatgcaagatgcatccacttatggtctgtatcaggtgaatagaaaataaccatttgccaagatttggaaagacagagagctctctggacacgggttcgatggagcttgcacctctctgagagctgaagtcaagtctcaccctctctccatactccctggcctagtgtgctctgggcagcaggtaggaagggacagggacctctcatctcaagggctgccccaaagggccctcagggaccatgggcctgttaggaccccagcaccccagtgtcgatcctggaagtcaaaggtcacgactgtggggggccagctcatgtcactgatggcagcaggcagggtttccagcatgggcggtgcgctgctgtctgatacttcattattgtgttgtcatcctagctgtataattaactatatccaaaagccggaagggaaacctgtagctctgcatatggagaaataaaacagagcatatgatgtatagagaaaatgaactgcacccagctgtgcactagagcgccatgttcttacccagcattccttgccacggtgtgaaccacagcagggaagctcgcatggttctacacgtcaccgaggcctggatgaaaatgtggcctcacgatgtgtatggttgggacccgccttctatttcattttatcttaacaccaacgaaagaaaaccgatttcagacgtttttcgctgatttatgatttattatatccctttctggcatcagccaaaatctcggtttttcctatgacatgaagtgatgtgactcttaccccgttgtcttaactcttggtatttttgaagcaggataagtcacacagcgggtcacgtagacacgtgctgttttgtgggacaggccctgggcctccttgctcctggcaggcctcaggtgccgcacgccgcgtcaccgtcaggactcgcaggctgtctggtcatttttgacgttgctgaaggtgagtgggaaaagctgggtgcactctgccccaaaggtggtgccctgtgtcccccacagatgatggccctccccagtgatggccggtcaccaggcacggctttcccctctgcatgaacagaacagaagcttctggctctccaaggcagcagcttggggcctggtggcccgggtgtgcgagccgcgagtgctttccaacagagggcaccagagccgtgcggtgtgctgtggagaccccagcaccacttcccctgctggaggatggatttgaaggagggtggctgggcggcaccccgatggctccctgtgctccctgagaccctacaggccgccctctgcagccacacctggaccggcaggcatgcacgctgcaggcaggctgcctccgctgtttgggtcccatgaacgatggaagcctcccagtgagatcttcaggtctctcatcaggcacaggctctgagggagggtcctggggccacatgactgcagccagcgagggccgagcccccagaatccaggtcagctcctggtggcaggggacctgcccgtcaaagccgtccaaggtgagccagcaaatccgtgtggcctgggagcacaggtgggcctgctcggaccatgcccttggtgcagtacaaaaccatgttctgcttcctctccgagggtttccgcggccctgtcagaacaggcttgatagatgcccggaggttccctctgacagccgcagcagcagggctcaaaccggcggggccgtgtaccgctccaaggacaagctctgccgaggccgacatgaaaagccagcacgcgggtcgcctagagccgctctgcctccaagctgcagcgccgttcgtgaaaatacagagggagagggaaggcacaaagaaatacaaaactattttccaaataaaaagaaatgtcgaaacaagaaagctgtgttcttcagccttggccctgggtccccctcaccctgtcgcatttcccagcactgtgatgctttgaaggccgacaccgagacccgccgtgcacgacagccacctcgcagccactcagaccggcggaccacgctcccctgtttccagagagatgagagagtcagaagtaatacttgtttttgcagaaccacacttgtggcaaaataagttgcctttggccacgcaggagactgagttttgtgaggggttgtccttctcccagtctctcttcccgctgggaacagaagtgtccagtaaacccacctgcattccacgtgtcctgtggccgcctccagtggtgagtctgtgaggccagagaagagaagctatgaccgtgcaaacatgcatgttatggtgttggctgatgcggaatcaggaagcttggaggagctcttgcatgaggaattgcagaacttaggggcatgaatttgcacttccagatgtgaaggttgcaggcaccgggcgttgcagaggaagtgggcaagctcaccttgtagagaacttacttggggtttgtaatttgtatacacttcttacccagcagaaacagcttactgaacacccctcgggtggctggatgatgtgctagcaactgaaaccatctctgcgacttaagcttctctggtgcgtcgcgccccacgtgcttctccagaatcccgtttggaggggccaagcaagtccaatatagataggtttatactgaagccatcagttttgtttcttacatcgtttgtaaacagcagtattatcctggaacttgtttcctacagaactgtaagataaggataaaatcccatccacctgaattgctgagaaacgtaagaggtggtgtgacccgctggagccctgtggagtggcggactctggggagtagctggtcagtgagattctcgcagcaatgtgaggcatcctcctcacagaaatgcatttacggatgttgctgtatcccctgacctggcctacaaggacagaacagccctgggggccacgtcccctcccggtctcatctgccgcttgctcaccttggctgtccatgcacccgtagctgtgaccatgggcggtggcgccagctcggacttcaccccgttggagcgggcaggatgtcacactgagctaatgtgagattgttcatgaagctacagatcttagtgctacttgggcttctcacagcacagaggaggattctgaagtgagagataaattggcttcatgtatgagcaagggggaaagtgttttattttactaggaagattttactgaaagaccaatcccgaaacggcccttgcttcggccacagctgctgggatgtcctcgggcctcccctgtttgtctgcatgtcttcttgttgccatcggggcagtggagtccccggaaacagtccaaggccctggggtgggggatttaaatttttggcttccctcagacctcagaccgtgtgggtttttttttctttctttctttcctgttcatgggcatttgattaaaagtttgtgtttaaaga

SEQ ID NO: 4 - protein, 148 aa, Homo sapiens
  MISC_FEATURE: MGMT Isoform X1
MQCTAWLNAYFHQPEAIEEFPVPALHHPVFQQESFTRQVLWKLLKVVKFGEVISYQQLAALAGNPKAARAVGGAMRGNPVPILIPCHRVVCSSGAVGNYSGGLAVKEWLLAHEGHRLGKPGLGGSSGLAGAWLKGAGATSGSPPAGRN

SEQ ID NO: 5 - DNA, 5985 bp, Homo sapiens
  misc_feature: GPRASP1 Transcript Variant 1
gcactttcgcgccgtatccctccgccccccttcccgacaccctcgcggagagcggttcttgccgcatcctgcgcagcccctgcccagtttggtgcagaggcgtggggggcgggactcgtctttgccattcggatcgctgggaaagcggtgggaatccaactgaagagcagccagaggagagctgaagagaggagggggaggccgatgacctgggctctgggcctctgaaggtctggcgtattctgacaggacacagtgagcatctgtagaggagaggcttgaaataaaggaggagcacgaatattccctggatttctggaggcctgctttaaggctggccagttctgcaagaaaggcaaggaggaggagactggctcacacctctggaggacccccttctgtcagctgtggggcttgacactacttgaacaagaaaaggagggggaaactgcaccacataagtgaagatccacctccagtggctgctctgctggtggtggggttgctgctgacaaccaccctcaacgggtctgcacccatccaggaaatctctgtcttcctcaagcttggttgtgcctgttctacactctatctgtattattgaattactgactgagactgtgtttgggaaggaggctgagtgactactggactggatattgactctaactcttgtttccaagcttatatcctcaatcacctaaagatcagagtgtgaagaaacaaacctgtgacagatctgtggttgaggtttagactgggggaggagtatagtactggactttctttgtaacttgtaccatgactggggcagagattgagtctggtgcccaggtcaagcctgaaaagaagcctggggaagaggttgtaggtggggctgagatagagaatgatgtccctctggtggtcagacccaaggttaggacccaggcccagataatgcctggggcaaggcccaagaataagtccaaggttatgcctggagcaagcaccaaagttgagacaagtgcagtgggtggggcacgccctaagagtaaggccaaggcaatacctgtttcacgatttaaggaagaagcccagatgtgggctcagcccaggtttggtgctgaaagattgtctaagacagagagaaactcccagaccaatatcatagcctctccacttgtcagtactgattctgtcttggttgctaaaacaaagtacctgtctgaggatagagaactggttaatacagacactgagagctttcctagaaggaaggcccattaccaagcaggattccagccttcttttaggtcaaaggaggagaccaatatggggtcctggtgctgtcctaggcctacatccaaacaagaagcctctcctaattctgatttcaaatgggtagacaaatctgtgagttccttgttctggagtggagatgaggtcactgcaaaatttcatcctgggaatagggtaaaagacagtaacagatccatgcacatggccaatcaagaggctaataccatgtctaggtcccaaactaaccaggagctctatattgcatctagttctggttctgaggatgagtctgttaagacaccctggttctgggccagagataaaaccaatacctggtctgggcccagggaagatcccaatagcaggtccaggtttaggtctaagaaagaagtctatgttgaatcaagttctggatctgagcatgaagaccatttggagtcctggtttggggctggaaaggaggccaaattcaggtccaaaatgagagctgggaaggaggccaataacagggccaggcacagggccaagcgagaagcttgcattgatttcatgcctgggtctatagatgtaattaaaaaagagtcctgtttctggcctgaagaaaatgctaataccttttcaaggcccatgatcaagaaagaggccagggccagagcaatgacaaaggaagaggccaaaaccaaggcccgagccagggccaagcaagaagccaggtcagaggaggaagccctcattgggacctggttctgggctacagacgagtccagcatggcagatgaagccagcatagagtccagtctacaagtggaggatgagtccataattgggagttggttctggactgaagaagaggccagtatggggactggggctagcagtaaatccagaccaaggactgatggggagcgtattggtgattccttatttggggctagggaaaagaccagtatgaaaactggggctgaggccacctctgaatctatactagcagctgatgatgaacaggtcattattggttcctggttctgggctggtgaagaggtcaaccaagaggctgaggaagagaccatttttgggtcgtggttctgggtcattgatgcggccagtgtggaatctggtgttggggtcagctgtgagtccaggacaaggtctgaggaagaagaggtcattggtccctggttttggtctggagaacaagttgatatagaggctggaatcggagaagaggccaggccaggagctgaagaagagacaatattcgggtcctggttttgggctgaaaaccagacctatatggattgtagggctgaaactagctgtgacaccatgcaaggggctgaggaggaggagcccattattgggtcctggttttggaccagagtagaagcttgtgtggagggtgatgtcaacagcaagtctagcctggaggacaaggaagaggccatgataccatgttttggagccaaagaagaggtcagtatgaagcatgggactggtgtcagatgcagatttatggcaggggctgaggagaccaataataagtcttgcttctgggcagaaaaagaaccctgtatgtatcctgccggtggaggaagttggaagtctaggccagaggaggaagaggacattgtcaattcgtggttctggtccagaaaatacacaaagccagaggccattatagggtcctggttatgggctacagaagagagtaatatagatgggactggagaaaaggccaagttactgactgaagaggagaccataatcaattcctggttctggaaagaagatgaagccatttcagaggctactgacagagaagagtccaggccagaagctgaggagggggacattattggttcttggttctgggctggagaagaggacagactagagccagctgctgagactagagaagaagacaggctagcagctgagaaagaaggtattgttgggtcctggtttggggccagagaagagaccattagaagagaggctgggtcttgcagcaaatccagtcctaaagctgaagaggaagaagtcattattgggtcctggttctgggaagaagaggccagtccggaggcagtggcaggagtcggctttgagtcaaagcctgggactgaggaggaagaaatcactgttgggtcctggttctggcctgaagaagaagccagtatacaggctggatctcaggcagtagaggaaatggagtcagagactgaagaggaaaccatttttgggtcctggttctgggatggaaaagaagtcagtgaagaagcaggaccatgctgtgtatccaagccagaggatgatgaagagatgattgttgagtcctggttctggtctagagacaaagccattaaggaaactggaactgtggccacctgtgagtccaagccagaaaatgaggaaggggccattgttgggtcttggtttgaggctgaagatgaggtagataacaggactgacaatggaagcaactgtgggtccaggacattagctgatgaagatgaggccatagtggggtcctggttctgggcaggagatgaggcccattttgaatcaaatcctagccccgtgttcagggccatttgcaggtccacgtgttcagttgaacaggagcctgatccttcacgcaggcctcagagttgggaggaggtcactgttcagttcaagcctggtccatggggtagggtcggcttcccatctataagcccctttagatttccgaaagaggcagcatctttattctgtgaaatgtttgggggcaaacccaggaacatggtacttagcccagaaggggaagatcaggaatctttgcttcagcctgatcagcctagtcctgagttcccatttcagtatgatccttcctacaggtcagtccaggaaattcgagagcatcttagggccaaggagagtacagagcctgagagttcatcctgtaactgcatacaatgtgagctgaaaattggttctgaagagtttgaagaactccttttattaatggaaaaaattcgggatccttttattcatgaaatatctaaaatcgcaatgggtatgagaagtgcttctcaatttacccgagatttcattcgagattcaggtgttgtctcacttattgaaaccttgcttaattatccgtcctcccgagttagaacaagttttttggaaaatatgattcgcatggccccaccttatccgaatctaaacataattcagacatacatatgtaaagtgtgtgaggaaacccttgcttatagcgtggattccccggaacagctgtctggaataaggatgattagacatctcactactactactgactatcacacactggttgccaattatatgtctgggtttctctccttattagctacaggcaatgccaaaacaaggtttcatgttttgaaaatgctactgaatttgtctgaaaatcttttcatgacaaaagaactactcagtgctgaagcagtgtcagaatttataggcctctttaacagggaagagacaaatgacaatattcaaattgttcttgcaatatttgagaatattggcaacaatatcaaaaaagaaacagtgttctctgatgatgatttcaatattgagccgcttatttctgcattccacaaagttgagaaatttgctaaggaactgcaaggcaaaacagacaatcaaaatgaccctgaaggggaccaagaaaattagtaatggttaattgctggcctcagattgtccttatgttcctgagttatgatccttgagtaatgctttgattttaatagttggttctgtgttgcaacatatatctttagtgctgacactaactttgtccaactctgtctgtaagctggagcatttttctgatgccagctgaatattagagctgaaaacacatttgttgatatttgtcttgtccacattgtgatgttcagtatttgagcttatagtgaactgagcaatcataaataagccacccttctgattgtcgttctactgtatatatatatatatttgagtgttgtttgtgtttcaataaagtcctatgttaaagttggcagaaatcacccttcttcttgaaattaaaatacagacccaatgataacacatgaacacacatagagataattaatatatgaataatatgttcctttcaaaagtttttttctcagctttaaacgcaaaaattaccaggggctgctatgtagggcatacttcagagaaaaagtagctacttcggttggactctgaagtttgggaaagatattggtggtgtaaaaaaaaaaaaaagcatggagttaggaaaaattttcctatgggagtgaggtatcctgtttgcctagagtgagtgagaggaatatattgagcaacatagaggatgacataagttttaaagagctgaggatttgcattttacagctcaaagtacaactgctgcttccatactacctaaagggcaaaagaataagacaggaaagcatagggatttgattgagtcaggaagacaatttaatcatgatggtaccatggtaagtgttaaggcaacatcatacagccattaaacatgatgatgtatagggttttaggaaaagccctgacatttttgatatttcatataatagagaacactggtgtcatacatgggaagtatgatcccatttgaaaaataaatgtgaataaattgaccaaaaaaaaaaaaaaaaa

SEQ ID NO: 6 - protein, 1395 aa, Homo sapiens
  MISC_FEATURE: GPRASP1 Transcript Variant 1
MTGAEIESGAQVKPEKKPGEEVVGGAEIENDVPLVVRPKVRTQAQIMPGARPKNKSKVMPGASTKVETSAVGGARPKSKAKAIPVSRFKEEAQMWAQPRFGAERLSKTERNSQTNIIASPLVSTDSVLVAKTKYLSEDRELVNTDTESFPRRKAHYQAGFQPSFRSKEETNMGSWCCPRPTSKQEASPNSDFKWVDKSVSSLFWSGDEVTAKFHPGNRVKDSNRSMHMANQEANTMSRSQTNQELYIASSSGSEDESVKTPWFWARDKTNTWSGPREDPNSRSRFRSKKEVYVESSSGSEHEDHLESWFGAGKEAKFRSKMRAGKEANNRARHRAKREACIDFMPGSIDVIKKESCFWPEENANTFSRPMIKKEARARAMTKEEAKTKARARAKQEARSEEEALIGTWFWATDESSMADEASIESSLQVEDESIIGSWFWTEEEASMGTGASSKSRPRTDGERIGDSLFGAREKTSMKTGAEATSESILAADDEQVIIGSWFWAGEEVNQEAEEETIFGSWFWVIDAASVESGVGVSCESRTRSEEEEVIGPWFWSGEQVDIEAGIGEEARPGAEEETIFGSWFWAENQTYMDCRAETSCDTMQGAEEEEPIIGSWFWTRVEACVEGDVNSKSSLEDKEEAMIPCFGAKEEVSMKHGTGVRCRFMAGAEETNNKSCFWAEKEPCMYPAGGGSWKSRPEEEEDIVNSWFWSRKYTKPEAIIGSWLWATEESNIDGTGEKAKLLTEEETIINSWFWKEDEAISEATDREESRPEAEEGDIIGSWFWAGEEDRLEPAAETREEDRLAAEKEGIVGSWFGAREETIRREAGSCSKSSPKAEEEEVIIGSWFWEEEASPEAVAGVGFESKPGTEEEEITVGSWFWPEEEASIQAGSQAVEEMESETEEETIFGSWFWDGKEVSEEAGPCCVSKPEDDEEMIVESWFWSRDKAIKETGTVATCESKPENEEGAIVGSWFEAEDEVDNRTDNGSNCGSRTLADEDEAIVGSWFWAGDEAHFESNPSPVFRAICRSTCSVEQEPDPSRRPQSWEEVTVQFKPGPWGRVGFPSISPFRFPKEAASLFCEMFGGKPRNMVLSPEGEDQESLLQPDQPSPEFPFQYDPSYRSVQEIREHLRAKESTEPESSSCNCIQCELKIGSEEFEELLLLMEKIRDPFIHEISKIAMGMRSASQFTRDFIRDSGVVSLIETLLNYPSSRVRTSFLENMIRMAPPYPNLNIIQTYICKVCEETLAYSVDSPEQLSGIRMIRHLTTTTDYHTLVANYMSGFLSLLATGNAKTRFHVLKMLLNLSENLFMTKELLSAEAVSEFIGLFNREETNDNIQIVLAIFENIGNNIKKETVFSDDDFNIEPLISAFHKVEKFAKELQGKTDNQNDPEGDQEN

SEQ ID NO: 7 - DNA, 5894 bp, Homo sapiens
  misc_feature: GPRASP1 Transcript Variant 2
gcactttcgcgccgtatccctccgccccccttcccgacaccctcgcggagagcggttcttgccgcatcctgcgcagcccctgcccagtttggtgcagaggcgtggggggcgggactcgtctttgccattcggatcgctgggaaagcggtgggaatccaactgaagagcagccagaggagagctgaagagaggagggggaggccgatgacctgggctctgggcctctgaaggcctgctttaaggctggccagttctgcaagaaaggcaaggaggaggagactggctcacacctctggaggacccccttctgtcagctgtggggcttgacactacttgaacaagaaaaggagggggaaactgcaccacataagtgaagatccacctccagtggctgctctgctggtggtggggttgctgctgacaaccaccctcaacgggtctgcacccatccaggaaatctctgtcttcctcaagcttggttgtgcctgttctacactctatctgtattattgaattactgactgagactgtgtttgggaaggaggctgagtgactactggactggatattgactctaactcttgtttccaagcttatatcctcaatcacctaaagatcagagtgtgaagaaacaaacctgtgacagatctgtggttgaggtttagactgggggaggagtatagtactggactttctttgtaacttgtaccatgactggggcagagattgagtctggtgcccaggtcaagcctgaaaagaagcctggggaagaggttgtaggtggggctgagatagagaatgatgtccctctggtggtcagacccaaggttaggacccaggcccagataatgcctggggcaaggcccaagaataagtccaaggttatgcctggagcaagcaccaaagttgagacaagtgcagtgggtggggcacgccctaagagtaaggccaaggcaatacctgtttcacgatttaaggaagaagcccagatgtgggctcagcccaggtttggtgctgaaagattgtctaagacagagagaaactcccagaccaatatcatagcctctccacttgtcagtactgattctgtcttggttgctaaaacaaagtacctgtctgaggatagagaactggttaatacagacactgagagctttcctagaaggaaggcccattaccaagcaggattccagccttcttttaggtcaaaggaggagaccaatatggggtcctggtgctgtcctaggcctacatccaaacaagaagcctctcctaattctgatttcaaatgggtagacaaatctgtgagttccttgttctggagtggagatgaggtcactgcaaaatttcatcctgggaatagggtaaaagacagtaacagatccatgcacatggccaatcaagaggctaataccatgtctaggtcccaaactaaccaggagctctatattgcatctagttctggttctgaggatgagtctgttaagacaccctggttctgggccagagataaaaccaatacctggtctgggcccagggaagatcccaatagcaggtccaggtttaggtctaagaaagaagtctatgttgaatcaagttctggatctgagcatgaagaccatttggagtcctggtttggggctggaaaggaggccaaattcaggtccaaaatgagagctgggaaggaggccaataacagggccaggcacagggccaagcgagaagcttgcattgatttcatgcctgggtctatagatgtaattaaaaaagagtcctgtttctggcctgaagaaaatgctaataccttttcaaggcccatgatcaagaaagaggccagggccagagcaatgacaaaggaagaggccaaaaccaaggcccgagccagggccaagcaagaagccaggtcagaggaggaagccctcattgggacctggttctgggctacagacgagtccagcatggcagatgaagccagcatagagtccagtctacaagtggaggatgagtccataattgggagttggttctggactgaagaagaggccagtatggggactggggctagcagtaaatccagaccaaggactgatggggagcgtattggtgattccttatttggggctagggaaaagaccagtatgaaaactggggctgaggccacctctgaatctatactagcagctgatgatgaacaggtcattattggttcctggttctgggctggtgaagaggtcaaccaagaggctgaggaagagaccatttttgggtcgtggttctgggtcattgatgcggccagtgtggaatctggtgttggggtcagctgtgagtccaggacaaggtctgaggaagaagaggtcattggtccctggttttggtctggagaacaagttgatatagaggctggaatcggagaagaggccaggccaggagctgaagaagagacaatattcgggtcctggttttgggctgaaaaccagacctatatggattgtagggctgaaactagctgtgacaccatgcaaggggctgaggaggaggagcccattattgggtcctggttttggaccagagtagaagcttgtgtggagggtgatgtcaacagcaagtctagcctggaggacaaggaagaggccatgataccatgttttggagccaaagaagaggtcagtatgaagcatgggactggtgtcagatgcagatttatggcaggggctgaggagaccaataataagtcttgcttctgggcagaaaaagaaccctgtatgtatcctgccggtggaggaagttggaagtctaggccagaggaggaagaggacattgtcaattcgtggttctggtccagaaaatacacaaagccagaggccattatagggtcctggttatgggctacagaagagagtaatatagatgggactggagaaaaggccaagttactgactgaagaggagaccataatcaattcctggttctggaaagaagatgaagccatttcagaggctactgacagagaagagtccaggccagaagctgaggagggggacattattggttcttggttctgggctggagaagaggacagactagagccagctgctgagactagagaagaagacaggctagcagctgagaaagaaggtattgttgggtcctggtttggggccagagaagagaccattagaagagaggctgggtcttgcagcaaatccagtcctaaagctgaagaggaagaagtcattattgggtcctggttctgggaagaagaggccagtccggaggcagtggcaggagtcggctttgagtcaaagcctgggactgaggaggaagaaatcactgttgggtcctggttctggcctgaagaagaagccagtatacaggctggatctcaggcagtagaggaaatggagtcagagactgaagaggaaaccatttttgggtcctggttctgggatggaaaagaagtcagtgaagaagcaggaccatgctgtgtatccaagccagaggatgatgaagagatgattgttgagtcctggttctggtctagagacaaagccattaaggaaactggaactgtggccacctgtgagtccaagccagaaaatgaggaaggggccattgttgggtcttggtttgaggctgaagatgaggtagataacaggactgacaatggaagcaactgtgggtccaggacattagctgatgaagatgaggccatagtggggtcctggttctgggcaggagatgaggcccattttgaatcaaatcctagccccgtgttcagggccatttgcaggtccacgtgttcagttgaacaggagcctgatccttcacgcaggcctcagagttgggaggaggtcactgttcagttcaagcctggtccatggggtagggtcggcttcccatctataagcccctttagatttccgaaagaggcagcatctttattctgtgaaatgtttgggggcaaacccaggaacatggtacttagcccagaaggggaagatcaggaatctttgcttcagcctgatcagcctagtcctgagttcccatttcagtatgatccttcctacaggtcagtccaggaaattcgagagcatcttagggccaaggagagtacagagcctgagagttcatcctgtaactgcatacaatgtgagctgaaaattggttctgaagagtttgaagaactccttttattaatggaaaaaattcgggatccttttattcatgaaatatctaaaatcgcaatgggtatgagaagtgcttctcaatttacccgagatttcattcgagattcaggtgttgtctcacttattgaaaccttgcttaattatccgtcctcccgagttagaacaagttttttggaaaatatgattcgcatggccccaccttatccgaatctaaacataattcagacatacatatgtaaagtgtgtgaggaaacccttgcttatagcgtggattccccggaacagctgtctggaataaggatgattagacatctcactactactactgactatcacacactggttgccaattatatgtctgggtttctctccttattagctacaggcaatgccaaaacaaggtttcatgttttgaaaatgctactgaatttgtctgaaaatcttttcatgacaaaagaactactcagtgctgaagcagtgtcagaatttataggcctctttaacagggaagagacaaatgacaatattcaaattgttcttgcaatatttgagaatattggcaacaatatcaaaaaagaaacagtgttctctgatgatgatttcaatattgagccgcttatttctgcattccacaaagttgagaaatttgctaaggaactgcaaggcaaaacagacaatcaaaatgaccctgaaggggaccaagaaaattagtaatggttaattgctggcctcagattgtccttatgttcctgagttatgatccttgagtaatgctttgattttaatagttggttctgtgttgcaacatatatctttagtgctgacactaactttgtccaactctgtctgtaagctggagcatttttctgatgccagctgaatattagagctgaaaacacatttgttgatatttgtcttgtccacattgtgatgttcagtatttgagcttatagtgaactgagcaatcataaataagccacccttctgattgtcgttctactgtatatatatatatatttgagtgttgtttgtgtttcaataaagtcctatgttaaagttggcagaaatcacccttcttcttgaaattaaaatacagacccaatgataacacatgaacacacatagagataattaatatatgaataatatgttcctttcaaaagtttttttctcagctttaaacgcaaaaattaccaggggctgctatgtagggcatacttcagagaaaaagtagctacttcggttggactctgaagtttgggaaagatattggtggtgtaaaaaaaaaaaaaagcatggagttaggaaaaattttcctatgggagtgaggtatcctgtttgcctagagtgagtgagaggaatatattgagcaacatagaggatgacataagttttaaagagctgaggatttgcattttacagctcaaagtacaactgctgcttccatactacctaaagggcaaaagaataagacaggaaagcatagggatttgattgagtcaggaagacaatttaatcatgatggtaccatggtaagtgttaaggcaacatcatacagccattaaacatgatgatgtatagggttttaggaaaagccctgacatttttgatatttcatataatagagaacactggtgtcatacatgggaagtatgatcccatttgaaaaataaatgtgaataaattgaccaaaaaaaaaaaaaaaaa

SEQ ID NO: 8 - protein, 1395 aa, Homo sapiens; identical to SEQ ID NO: 6
  MISC_FEATURE: GPRASP1 Transcript Variant 2

SEQ ID NO: 9 - DNA, 5826 bp, Homo sapiens
  misc_feature: GPRASP1 Transcript Variant 3
gcactttcgcgccgtatccctccgccccccttcccgacaccctcgcggagagcggttcttgccgcatcctgcgcagcccctgcccagtttggtgcagaggcgtggggggcgggactcgtctttgccattcggatcgctgggaaagcggtgggaatccaactgaagagcagccagaggagagctgaagagaggagggggaggccgatgacctgggctctgggcctctgaaggacccccttctgtcagctgtggggcttgacactacttgaacaagaaaaggagggggaaactgcaccacataagtgaagatccacctccagtggctgctctgctggtggtggggttgctgctgacaaccaccctcaacgggtctgcacccatccaggaaatctctgtcttcctcaagcttggttgtgcctgttctacactctatctgtattattgaattactgactgagactgtgtttgggaaggaggctgagtgactactggactggatattgactctaactcttgtttccaagcttatatcctcaatcacctaaagatcagagtgtgaagaaacaaacctgtgacagatctgtggttgaggtttagactgggggaggagtatagtactggactttctttgtaacttgtaccatgactggggcagagattgagtctggtgcccaggtcaagcctgaaaagaagcctggggaagaggttgtaggtggggctgagatagagaatgatgtccctctggtggtcagacccaaggttaggacccaggcccagataatgcctggggcaaggcccaagaataagtccaaggttatgcctggagcaagcaccaaagttgagacaagtgcagtgggtggggcacgccctaagagtaaggccaaggcaatacctgtttcacgatttaaggaagaagcccagatgtgggctcagcccaggtttggtgctgaaagattgtctaagacagagagaaactcccagaccaatatcatagcctctccacttgtcagtactgattctgtcttggttgctaaaacaaagtacctgtctgaggatagagaactggttaatacagacactgagagctttcctagaaggaaggcccattaccaagcaggattccagccttcttttaggtcaaaggaggagaccaatatggggtcctggtgctgtcctaggcctacatccaaacaagaagcctctcctaattctgatttcaaatgggtagacaaatctgtgagttccttgttctggagtggagatgaggtcactgcaaaatttcatcctgggaatagggtaaaagacagtaacagatccatgcacatggccaatcaagaggctaataccatgtctaggtcccaaactaaccaggagctctatattgcatctagttctggttctgaggatgagtctgttaagacaccctggttctgggccagagataaaaccaatacctggtctgggcccagggaagatcccaatagcaggtccaggtttaggtctaagaaagaagtctatgttgaatcaagttctggatctgagcatgaagaccatttggagtcctggtttggggctggaaaggaggccaaattcaggtccaaaatgagagctgggaaggaggccaataacagggccaggcacagggccaagcgagaagcttgcattgatttcatgcctgggtctatagatgtaattaaaaaagagtcctgtttctggcctgaagaaaatgctaataccttttcaaggcccatgatcaagaaagaggccagggccagagcaatgacaaaggaagaggccaaaaccaaggcccgagccagggccaagcaagaagccaggtcagaggaggaagccctcattgggacctggttctgggctacagacgagtccagcatggcagatgaagccagcatagagtccagtctacaagtggaggatgagtccataattgggagttggttctggactgaagaagaggccagtatggggactggggctagcagtaaatccagaccaaggactgatggggagcgtattggtgattccttatttggggctagggaaaagaccagtatgaaaactggggctgaggccacctctgaatctatactagcagctgatgatgaacaggtcattattggttcctggttctgggctggtgaagaggtcaaccaagaggctgaggaagagaccatttttgggtcgtggttctgggtcattgatgcggccagtgtggaatctggtgttggggtcagctgtgagtccaggacaaggtctgaggaagaagaggtcattggtccctggttttggtctggagaacaagttgatatagaggctggaatcggagaagaggccaggccaggagctgaagaagagacaatattcgggtcctggttttgggctgaaaaccagacctatatggattgtagggctgaaactagctgtgacaccatgcaaggggctgaggaggaggagcccattattgggtcctggttttggaccagagtagaagcttgtgtggagggtgatgtcaacagcaagtctagcctggaggacaaggaagaggccatgataccatgttttggagccaaagaagaggtcagtatgaagcatgggactggtgtcagatgcagatttatggcaggggctgaggagaccaataataagtcttgcttctgggcagaaaaagaaccctgtatgtatcctgccggtggaggaagttggaagtctaggccagaggaggaagaggacattgtcaattcgtggttctggtccagaaaatacacaaagccagaggccattatagggtcctggttatgggctacagaagagagtaatatagatgggactggagaaaaggccaagttactgactgaagaggagaccataatcaattcctggttctggaaagaagatgaagccatttcagaggctactgacagagaagagtccaggccagaagctgaggagggggacattattggttcttggttctgggctggagaagaggacagactagagccagctgctgagactagagaagaagacaggctagcagctgagaaagaaggtattgttgggtcctggtttggggccagagaagagaccattagaagagaggctgggtcttgcagcaaatccagtcctaaagctgaagaggaagaagtcattattgggtcctggttctgggaagaagaggccagtccggaggcagtggcaggagtcggctttgagtcaaagcctgggactgaggaggaagaaatcactgttgggtcctggttctggcctgaagaagaagccagtatacaggctggatctcaggcagtagaggaaatggagtcagagactgaagaggaaaccatttttgggtcctggttctgggatggaaaagaagtcagtgaagaagcaggaccatgctgtgtatccaagccagaggatgatgaagagatgattgttgagtcctggttctggtctagagacaaagccattaaggaaactggaactgtggccacctgtgagtccaagccagaaaatgaggaaggggccattgttgggtcttggtttgaggctgaagatgaggtagataacaggactgacaatggaagcaactgtgggtccaggacattagctgatgaagatgaggccatagtggggtcctggttctgggcaggagatgaggcccattttgaatcaaatcctagccccgtgttcagggccatttgcaggtccacgtgttcagttgaacaggagcctgatccttcacgcaggcctcagagttgggaggaggtcactgttcagttcaagcctggtccatggggtagggtcggcttcccatctataagcccctttagatttccgaaagaggcagcatctttattctgtgaaatgtttgggggcaaacccaggaacatggtacttagcccagaaggggaagatcaggaatctttgcttcagcctgatcagcctagtcctgagttcccatttcagtatgatccttcctacaggtcagtccaggaaattcgagagcatcttagggccaaggagagtacagagcctgagagttcatcctgtaactgcatacaatgtgagctgaaaattggttctgaagagtttgaagaactccttttattaatggaaaaaattcgggatccttttattcatgaaatatctaaaatcgcaatgggtatgagaagtgcttctcaatttacccgagatttcattcgagattcaggtgttgtctcacttattgaaaccttgcttaattatccgtcctcccgagttagaacaagttttttggaaaatatgattcgcatggccccaccttatccgaatctaaacataattcagacatacatatgtaaagtgtgtgaggaaacccttgcttatagcgtggattccccggaacagctgtctggaataaggatgattagacatctcactactactactgactatcacacactggttgccaattatatgtctgggtttctctccttattagctacaggcaatgccaaaacaaggtttcatgttttgaaaatgctactgaatttgtctgaaaatcttttcatgacaaaagaactactcagtgctgaagcagtgtcagaatttataggcctctttaacagggaagagacaaatgacaatattcaaattgttcttgcaatatttgagaatattggcaacaatatcaaaaaagaaacagtgttctctgatgatgatttcaatattgagccgcttatttctgcattccacaaagttgagaaatttgctaaggaactgcaaggcaaaacagacaatcaaaatgaccctgaaggggaccaagaaaattagtaatggttaattgctggcctcagattgtccttatgttcctgagttatgatccttgagtaatgctttgattttaatagttggttctgtgttgcaacatatatctttagtgctgacactaactttgtccaactctgtctgtaagctggagcatttttctgatgccagctgaatattagagctgaaaacacatttgttgatatttgtcttgtccacattgtgatgttcagtatttgagcttatagtgaactgagcaatcataaataagccacccttctgattgtcgttctactgtatatatatatatatttgagtgttgtttgtgtttcaataaagtcctatgttaaagttggcagaaatcacccttcttcttgaaattaaaatacagacccaatgataacacatgaacacacatagagataattaatatatgaataatatgttcctttcaaaagtttttttctcagctttaaacgcaaaaattaccaggggctgctatgtagggcatacttcagagaaaaagtagctacttcggttggactctgaagtttgggaaagatattggtggtgtaaaaaaaaaaaaaagcatggagttaggaaaaattttcctatgggagtgaggtatcctgtttgcctagagtgagtgagaggaatatattgagcaacatagaggatgacataagttttaaagagctgaggatttgcattttacagctcaaagtacaactgctgcttccatactacctaaagggcaaaagaataagacaggaaagcatagggatttgattgagtcaggaagacaatttaatcatgatggtaccatggtaagtgttaaggcaacatcatacagccattaaacatgatgatgtatagggttttaggaaaagccctgacatttttgatatttcatataatagagaacactggtgtcatacatgggaagtatgatcccatttgaaaaataaatgtgaataaattgaccaaaaaaaaaaaaaaaaa

SEQ ID NO: 10 - protein, 1395 aa, Homo sapiens; identical to SEQ ID NO: 6
  MISC_FEATURE: GPRASP1 Transcript Variant 3

SEQ ID NO: 11 - DNA, 5997 bp, Homo sapiens
  misc_feature: GPRASP1 Transcript Variant 4
gacaccctcgcggagagcggttcttgccgcatcctgcgcagcccctgcccagtttggtgcagaggcgtggggggcgggactcgtctttgccattcggatcgctgggaaagcggtgggaatccaactgaagagcagccagaggagagctgaagagaggagggggaggccgatgacctgggctctgggcctctgaagcctggcaacgaggagacgatccgttcggagccgggcgctagagagaggtctggcgtattctgacaggacacagtgagcatctgtagaggagaggcttgaaataaaggaggagcacgaatattccctggatttctggaggcctgctttaaggctggccagttctgcaagaaaggcaaggaggaggagactggctcacacctctggaggacccccttctgtcagctgtggggcttgacactacttgaacaagaaaaggagggggaaactgcaccacataagtgaagatccacctccagtggctgctctgctggtggtggggttgctgctgacaaccaccctcaacgggtctgcacccatccaggaaatctctgtcttcctcaagcttggttgtgcctgttctacactctatctgtattattgaattactgactgagactgtgtttgggaaggaggctgagtgactactggactggatattgactctaactcttgtttccaagcttatatcctcaatcacctaaagatcagagtgtgaagaaacaaacctgtgacagatctgtggttgaggtttagactgggggaggagtatagtactggactttctttgtaacttgtaccatgactggggcagagattgagtctggtgcccaggtcaagcctgaaaagaagcctggggaagaggttgtaggtggggctgagatagagaatgatgtccctctggtggtcagacccaaggttaggacccaggcccagataatgcctggggcaaggcccaagaataagtccaaggttatgcctggagcaagcaccaaagttgagacaagtgcagtgggtggggcacgccctaagagtaaggccaaggcaatacctgtttcacgatttaaggaagaagcccagatgtgggctcagcccaggtttggtgctgaaagattgtctaagacagagagaaactcccagaccaatatcatagcctctccacttgtcagtactgattctgtcttggttgctaaaacaaagtacctgtctgaggatagagaactggttaatacagacactgagagctttcctagaaggaaggcccattaccaagcaggattccagccttcttttaggtcaaaggaggagaccaatatggggtcctggtgctgtcctaggcctacatccaaacaagaagcctctcctaattctgatttcaaatgggtagacaaatctgtgagttccttgttctggagtggagatgaggtcactgcaaaatttcatcctgggaatagggtaaaagacagtaacagatccatgcacatggccaatcaagaggctaataccatgtctaggtcccaaactaaccaggagctctatattgcatctagttctggttctgaggatgagtctgttaagacaccctggttctgggccagagataaaaccaatacctggtctgggcccagggaagatcccaatagcaggtccaggtttaggtctaagaaagaagtctatgttgaatcaagttctggatctgagcatgaagaccatttggagtcctggtttggggctggaaaggaggccaaattcaggtccaaaatgagagctgggaaggaggccaataacagggccaggcacagggccaagcgagaagcttgcattgatttcatgcctgggtctatagatgtaattaaaaaagagtcctgtttctggcctgaagaaaatgctaataccttttcaaggcccatgatcaagaaagaggccagggccagagcaatgacaaaggaagaggccaaaaccaaggcccgagccagggccaagcaagaagccaggtcagaggaggaagccctcattgggacctggttctgggctacagacgagtccagcatggcagatgaagccagcatagagtccagtctacaagtggaggatgagtccataattgggagttggttctggactgaagaagaggccagtatggggactggggctagcagtaaatccagaccaaggactgatggggagcgtattggtgattccttatttggggctagggaaaagaccagtatgaaaactggggctgaggccacctctgaatctatactagcagctgatgatgaacaggtcattattggttcctggttctgggctggtgaagaggtcaaccaagaggctgaggaagagaccatttttgggtcgtggttctgggtcattgatgcggccagtgtggaatctggtgttggggtcagctgtgagtccaggacaaggtctgaggaagaagaggtcattggtccctggttttggtctggagaacaagttgatatagaggctggaatcggagaagaggccaggccaggagctgaagaagagacaatattcgggtcctggttttgggctgaaaaccagacctatatggattgtagggctgaaactagctgtgacaccatgcaaggggctgaggaggaggagcccattattgggtcctggttttggaccagagtagaagcttgtgtggagggtgatgtcaacagcaagtctagcctggaggacaaggaagaggccatgataccatgttttggagccaaagaagaggtcagtatgaagcatgggactggtgtcagatgcagatttatggcaggggctgaggagaccaataataagtcttgcttctgggcagaaaaagaaccctgtatgtatcctgccggtggaggaagttggaagtctaggccagaggaggaagaggacattgtcaattcgtggttctggtccagaaaatacacaaagccagaggccattatagggtcctggttatgggctacagaagagagtaatatagatgggactggagaaaaggccaagttactgactgaagaggagaccataatcaattcctggttctggaaagaagatgaagccatttcagaggctactgacagagaagagtccaggccagaagctgaggagggggacattattggttcttggttctgggctggagaagaggacagactagagccagctgctgagactagagaagaagacaggctagcagctgagaaagaaggtattgttgggtcctggtttggggccagagaagagaccattagaagagaggctgggtcttgcagcaaatccagtcctaaagctgaagaggaagaagtcattattgggtcctggttctgggaagaagaggccagtccggaggcagtggcaggagtcggctttgagtcaaagcctgggactgaggaggaagaaatcactgttgggtcctggttctggcctgaagaagaagccagtatacaggctggatctcaggcagtagaggaaatggagtcagagactgaagaggaaaccatttttgggtcctggttctgggatggaaaagaagtcagtgaagaagcaggaccatgctgtgtatccaagccagaggatgatgaagagatgattgttgagtcctggttctggtctagagacaaagccattaaggaaactggaactgtggccacctgtgagtccaagccagaaaatgaggaaggggccattgttgggtcttggtttgaggctgaagatgaggtagataacaggactgacaatggaagcaactgtgggtccaggacattagctgatgaagatgaggccatagtggggtcctggttctgggcaggagatgaggcccattttgaatcaaatcctagccccgtgttcagggccatttgcaggtccacgtgttcagttgaacaggagcctgatccttcacgcaggcctcagagttgggaggaggtcactgttcagttcaagcctggtccatggggtagggtcggcttcccatctataagcccctttagatttccgaaagaggcagcatctttattctgtgaaatgtttgggggcaaacccaggaacatggtacttagcccagaaggggaagatcaggaatctttgcttcagcctgatcagcctagtcctgagttcccatttcagtatgatccttcctacaggtcagtccaggaaattcgagagcatcttagggccaaggagagtacagagcctgagagttcatcctgtaactgcatacaatgtgagctgaaaattggttctgaagagtttgaagaactccttttattaatggaaaaaattcgggatccttttattcatgaaatatctaaaatcgcaatgggtatgagaagtgcttctcaatttacccgagatttcattcgagattcaggtgttgtctcacttattgaaaccttgcttaattatccgtcctcccgagttagaacaagttttttggaaaatatgattcgcatggccccaccttatccgaatctaaacataattcagacatacatatgtaaagtgtgtgaggaaacccttgcttatagcgtggattccccggaacagctgtctggaataaggatgattagacatctcactactactactgactatcacacactggttgccaattatatgtctgggtttctctccttattagctacaggcaatgccaaaacaaggtttcatgttttgaaaatgctactgaatttgtctgaaaatcttttcatgacaaaagaactactcagtgctgaagcagtgtcagaatttataggcctctttaacagggaagagacaaatgacaatattcaaattgttcttgcaatatttgagaatattggcaacaatatcaaaaaagaaacagtgttctctgatgatgatttcaatattgagccgcttatttctgcattccacaaagttgagaaatttgctaaggaactgcaaggcaaaacagacaatcaaaatgaccctgaaggggaccaagaaaattagtaatggttaattgctggcctcagattgtccttatgttcctgagttatgatccttgagtaatgctttgattttaatagttggttctgtgttgcaacatatatctttagtgctgacactaactttgtccaactctgtctgtaagctggagcatttttctgatgccagctgaatattagagctgaaaacacatttgttgatatttgtcttgtccacattgtgatgttcagtatttgagcttatagtgaactgagcaatcataaataagccacccttctgattgtcgttctactgtatatatatatatatttgagtgttgtttgtgtttcaataaagtcctatgttaaagttggcagaaatcacccttcttcttgaaattaaaatacagacccaatgataacacatgaacacacatagagataattaatatatgaataatatgttcctttcaaaagtttttttctcagctttaaacgcaaaaattaccaggggctgctatgtagggcatacttcagagaaaaagtagctacttcggttggactctgaagtttgggaaagatattggtggtgtaaaaaaaaaaaaaagcatggagttaggaaaaattttcctatgggagtgaggtatcctgtttgcctagagtgagtgagaggaatatattgagcaacatagaggatgacataagttttaaagagctgaggatttgcattttacagctcaaagtacaactgctgcttccatactacctaaagggcaaaagaataagacaggaaagcatagggatttgattgagtcaggaagacaatttaatcatgatggtaccatggtaagtgttaaggcaacatcatacagccattaaacatgatgatgtatagggttttaggaaaagccctgacatttttgatatttcatataatagagaacactggtgtcatacatgggaagtatgatcccatttgaaaaataaatgtgaataaattgaccaaaaaaaaaaaaaaaaa

SEQ ID NO: 12 - protein, 1395 aa, Homo sapiens; identical to SEQ ID NO: 6
  MISC_FEATURE: GPRASP1 Transcript Variant 4

SEQ ID NO: 13 - DNA, 2079 bp, Homo sapiens
  misc_feature: CHGA Transcript Variant 1
gtcatttccggggtcggggtatataagcggggcgcgagggcgctgctgctgccaccgctcctgccactgcagtgctcgagccccgtgcaggggagcttgcgggaggatcgaccgacagacggacgcacgccgaggcactgcgcccccagccccgcgccggtgccaccgcagcccgaccccggccgccagtccagccgcccctcgcccggtgcctaggtgcccggccccacaccgccagctgctcggcgcccgggtccgccatgcgctccgccgctgtcctggctcttctgctctgcgccgggcaagtcactgcgctccctgtgaacagccctatgaataaaggggataccgaggtgatgaaatgcatcgttgaggtcatctccgacacactttccaagcccagccccatgcctgtcagccaggaatgttttgagacactccgaggagatgaacggatcctttccattctgagacatcagaatttactgaaggagctccaagacctcgctctccaaggcgccaaggagagggcacatcagcagaagaaacacagcggttttgaagatgaactctcagaggttcttgagaaccagagcagccaggccgagctgaaagaggcggtggaagagccatcatccaaggatgttatggagaaaagagaggattccaaggaggcagagaaaagtggtgaagccacagacggagccaggccccaggccctcccggagcccatgcaggagtccaaggctgaggggaacaatcaggcccctggggaggaagaggaggaggaggaggaggccaccaacacccaccctccagccagcctccccagccagaaatacccaggcccacaggccgagggggacagtgagggcctctctcagggtctggtggacagagagaagggcctgagtgcagagccagggtggcaggcaaagagagaagaggaggaggaggaggaggaggaggctgaggctggagaggaggctgtccccgaggaagaaggccccactgtagtgctgaacccccacccgagccttggctacaaggagatccggaaaggcgagagtcggtcggaggctctggctgtggatggagctgggaagcctggggctgaggaggctcaggaccccgaagggaagggagaacaggagcactcccagcagaaagaggaggaggaggagatggcagtggtcccgcaaggcctcttccggggtgggaagagcggagagctggagcaggaggaggagcggctctccaaggagtgggaggactccaaacgctggagcaagatggaccagctggccaaggagctgacggctgagaagcggctggaggggcaggaggaggaggaggacaaccgggacagttccatgaagctctccttccgggcccgggcctacggcttcaggggccctgggccgcagctgcgacgaggctggaggccatcctcccgggaggacagccttgaggcgggcctgcccctccaggtccgaggctaccccgaggagaagaaagaggaggagggcagcgcaaaccgcagaccagaggaccaggagctggagagcctgtcggccattgaagcagagctggagaaagtggcccaccagctgcaggcactacggcggggctgagacaccggctggcagggctggccccagggcaccctgtggccctggctctgctgtccccttggcaggtcctggccagatggcccggatgctgcttccggtagggaggcagcctccagcctgcccaagcccaggccaccctatcgccccctacgcgccttgtctcctactcctgactcctacctgccctggaacatcctttgcagggcagccccacaactttaaacattgacgattccttctctgaacacaggcagctttctagaagtttcccttcctccatcctatccactgggcacaactgcaataacttctgaccttttggtgaaagctgagaactcctgactgtaacatattctgtatgaactttatctaaagaaaaataaatctgttctgggctctttcctctgaaaaaaaaaaaaaaaaaaaaaaaaaaaaaaaaaaaaaa

SEQ ID NO: 14 - protein, 457 aa, Homo sapiens
  MISC_FEATURE: CHGA Transcript Variant 1
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 15 - DNA, 1626 bp, Homo sapiens
  misc_feature: CHGA Transcript Variant 2
gtcatttccggggtcggggtatataagcggggcgcgagggcgctgctgctgccaccgctcctgccactgcagtgctcgagccccgtgcaggggagcttgcgggaggatcgaccgacagacggacgcacgccgaggcactgcgcccccagccccgcgccggtgccaccgcagcccgaccccggccgccagtccagccgcccctcgcccggtgcctaggtgcccggccccacaccgccagctgctcggcgcccgggtccgccatgcgctccgccgctgtcctggctcttctgctctgcgccgggcaagtcactgcgctccctgtgaacagccctatgaataaaggggataccgaggtgatgaaatgcatcgttgaggtcatctccgacacactttccaagcccagccccatgcctgtcagccaggaatgttttgagacactccgaggagatgaacggatcctttccattctgagacatcagaatttactgaaggagctccaagacctcgctctccaaggcgccaaggagagggcacatcagcagaagaaacacagcggttttgaagatgaactctcagaggttcttgagaaccagagcagccaggccgagctgaaaggtcggtcggaggctctggctgtggatggagctgggaagcctggggctgaggaggctcaggaccccgaagggaagggagaacaggagcactcccagcagaaagaggaggaggaggagatggcagtggtcccgcaaggcctcttccggggtgggaagagcggagagctggagcaggaggaggagcggctctccaaggagtgggaggactccaaacgctggagcaagatggaccagctggccaaggagctgacggctgagaagcggctggaggggcaggaggaggaggaggacaaccgggacagttccatgaagctctccttccgggcccgggcctacggcttcaggggccctgggccgcagctgcgacgaggctggaggccatcctcccgggaggacagccttgaggcgggcctgcccctccaggtccgaggctaccccgaggagaagaaagaggaggagggcagcgcaaaccgcagaccagaggaccaggagctggagagcctgtcggccattgaagcagagctggagaaagtggcccaccagctgcaggcactacggcggggctgagacaccggctggcagggctggccccagggcaccctgtggccctggctctgctgtccccttggcaggtcctggccagatggcccggatgctgcttccggtagggaggcagcctccagcctgcccaagcccaggccaccctatcgccccctacgcgccttgtctcctactcctgactcctacctgccctggaacatcctttgcagggcagccccacaactttaaacattgacgattccttctctgaacacaggcagctttctagaagtttcccttcctccatcctatccactgggcacaactgcaataacttctgaccttttggtgaaagctgagaactcctgactgtaacatattctgtatgaactttatctaaagaaaaataaatctgttctgggctctttcctctgaaaaaaaaaaaaaaaaaaaaaaaaaaaaaaaaaaaaaa

SEQ ID NO: 16 - protein, 306 aa, Homo sapiens
  MISC_FEATURE: CHGA Transcript Variant 2
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKGRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 17 - DNA, 5854 bp, Homo sapiens
  misc_feature: MAPK8 Isoform Beta2
gacgtgcgcgggcgtgcgcggtgacggcccgcgtctctgttactcagccgagcggccgaggccggacgacgcggcttggattgcggagccgcgagcagcgctgggtaacggccgcggcgaccaccccggacggcccctgtccccgctggcgggcttccctgtcgccgttcgctgcgctgccggcttcttggtgaatttttggatgaagccattaaattaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgttgacatttggtcagttgggtgcatcatgggagaaatgatcaaaggtggtgttttgttcccaggtacagatcatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgtagaattcattttgtagtaaagtagtttattttttttaatttcaagtgatgtaatttaaaacctaagttgtgtttcaaaacagcaacaaaactgtattgtattttttttgctgtaattaactgtataatgtaaacctaattattttatcatggtttaaattttttgcatatttgctttatcttatgctgctgatttttttaactgaatttgtaagattttgtttatcaaagcaactattatgtggtgacttgcctatatcatgaattatttaagatttttatagttttttttaattagaatttatttcagatgttttgttcatgatactatccttcagggttatgtgcttatcaatgaaataaccccagaggagtgagggaaaataacttgtagccagttatattcaggaataactactgtaaatgatgaacgtgttaggagacctccaatatttgctacttgccaatcctaatttagttacaagaattggtaggcaatcctacttaattttggcaaaagccccgtcatctaaatggcagaataactcagagcatgtctttgaagatgctgggcgtctaccaccaccttatgtccccaccctacccaacaaaaataagtaaaaagaatatggtgtattctacaaatttgtggcatgctcaaagtttatgatcacataaaggcaagaggatacttcatgaataatacatttcaatgcaaataaacagatggttcacttctactagctatgagcctgtttttgtatacactgagttaatctactcaggctgtaggtcccagcaatgttctagagtctggtctttccctttcctgcagcttcgggtccttggacctttcctgtttcctattacttggagtgtctgtcagttgagcaccagttgttctggtgtttcatttgattctacttgtagcataatcatttatacgagctattgggaggttccaaaccctacctagatttgtgtaggtgatgtatcaaatgagcaatataccgttcatctgaaaatagtagcacacagccatatataggatatcattttctaaggactgtttcttcacattgagcagagcaggcataaatggtggttatttagtctaagtcttttatttttttatacctgattttcaacataacacgcaatgtggatgtcgagtagtgttaagaatggtgctgctcctgacaagtgtatgttaactgtttacattttctatctgtagaattatttctctattactgaacttttcctaagtaaaatgtctttgaagtctcgttatttctgaaatacgttgtctgtaatagacccaggcaccttttaaattatctctggaacaagagggatttcatgtaatgaactaggaaatgcatactcacataagcaacaaggttctaggcagaaagccccttggaatttgtgaccaacaggagcaagaacaggtgcggctcaacatgcaatgtctgaaaatttgcttggcattttattcatatatttagtgcaaaattatttttgagtgagatattttacatcactgttaatgtgcaatatttaagattaaaatacattagcttttttatatactttgaagtagcaagtttgttttcgatggcttagagtcatgatttccagcttcccagcctttttatcagtcccttttctaatacaacaaggtgcattaatttgattaggcaaattagagttctaagacacttcttgaattgtagacagaaaatattggattcacaatttcagcagaaatttgagaatgagtgtgtttatattaatttcacaattagctgtattttctgtagcatagattatgtcactgttgcactttcacagcagacatgctttcagaaggttctcatattttatgtttgattgctgataagccatctctattgatacagattttggttaagtaaggaaaaccaggtgtgtgtctgtatcatttattgtaaatgccagctgccacttgccaaccatcatgttcagttcaattcaaagaaaacaaactctcattacttagtgtaaactaaaatacttaacaaattatatcctaaaaacaaggtctctttgttaaatgttgcatgccctaggttttaaattactacatccaaatacagttttcgtcttaaatttgttaagctaaatatatgttggttctttttattttggaatcctttaagcatcttaaacattttttttttgaagagaagttacaaataacatttctatcaggtagtacttgtatgaaaccacctttcttattctataattttgatttttcaattttatatacttaatatactcactgtcttactatcagaaagttattttgaccaagatttttattatcttcatagattcagaaagagatgctaattctgtaccaatgtcttcctggttactattctcttccctctaatatatactggccatttgtaaaaccattgtgttgttgggatcacttagttatactatacgcagatagagcatctcaactctgtcatagtgtttgctgaacagttttcagtgtcatgcacctttggctgctaattgttcctgacgtgcactcttccgagttggtaaaggcacagtgtgttcatgccagacttctaagagaaacaccagcctcttaaatcagaagcctacacacaacccccttaacaatccaaagaagcttgatggtgtgcaaagaagcatcctgccagccttgtcattgttctgttctatgctaatcctgctgtgttgtctaaaagatggagggaagaggacatcagtgtctgatagtgaaatcatcagcaggaaagtgaagctctttccttggttacagataagacttggtttacactattggccagtatctgctaaacatatgaagacttaactattcagtgttgcctaggcattcgcctgcacaacattttgaggttagaacatagaatattttcagaaatactgttgtagtttgtgagtgttgttcattagttacacattagctatagagtggatgcatgaagccccatgacaccagtaaacttctcttaccagtaggtaaaccaaacaccattctgtcattagcagccctcttaaatgttgcctctccgtatcctgttgcatttttgtgtgcattgtgtttctactgatctctcttaggtttttacggaatcaaaggaaactaatttttccttaatagcaagaaagatgaagaggtaaagggcattgaagcagaaatgtatagtttggggtacgattagaaaactcgtaaggaaaacagaagtcctaatttcaaactgactgctcttcgttaagtgctcttaaggagagtctagtaacagtaacactttctggccatttctagtttagattctcttcgttactgaaacttttgagaaatattacctgtggattaattttgcacaatgttctattctcataatgacttacaaattaaactaggtttttattgaactacctcacactaattttctatgctttcccaagtaagctgttgccctgttagatctttactgagtgaattataaatgtgtgttaaatactttctagccaatgttgacacaataccagtaagtatgtaaagtatataccttacatcagtaagagacacgtgtaaaatctttgactgtatgtcttgcaaaattgtgctcgttgacattattactgtttttgtaagtagaaacctgctcgtgatatcggtccatttacattttacaaaaggagtaaatcttagtaaaaattttacgaagaaataaattacttttgtaggcccaatatttggtatatttttgagaagctgttaatcttttagctgaataatgaagttagactgaattacgtgtctccctggactgtgacatctattttctcattacagtttatcctggtcagcagggtgtcacacctggaaacctgagtatgatagctgacatttgcttttctccctctgcgatgtcattcctcctccattcctctccttccctgtgttccgttccctctcctttcctctagacaaaacaaaatggggcactttttagggaatgctgagatcattattgtggtttttcatcattcatgccctagtcattaaacatgcaccactggaatgtaaacaatgttatctagtatgtcaattggttataatattttaaataaaaaagaaaaaagtggtatgaaaattatgaaaaaaaaaa

SEQ ID NO: 18 - protein, 427 aa, Homo sapiens
  MISC_FEATURE: MAPK8 Isoform Beta2
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMIKGGVLFPGTDHIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 19 - DNA, 5448 bp, Homo sapiens
  misc_feature: MAPK8 Isoform 5
cttcttggtgaatttttggatgaagccattaaattaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttagcacaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgtagaattcattttgtagtaaagtagtttattttttttaatttcaagtgatgtaatttaaaacctaagttgtgtttcaaaacagcaacaaaactgtattgtattttttttgctgtaattaactgtataatgtaaacctaattattttatcatggtttaaattttttgcatatttgctttatcttatgctgctgatttttttaactgaatttgtaagattttgtttatcaaagcaactattatgtggtgacttgcctatatcatgaattatttaagatttttatagttttttttaattagaatttatttcagatgttttgttcatgatactatccttcagggttatgtgcttatcaatgaaataaccccagaggagtgagggaaaataacttgtagccagttatattcaggaataactactgtaaatgatgaacgtgttaggagacctccaatatttgctacttgccaatcctaatttagttacaagaattggtaggcaatcctacttaattttggcaaaagccccgtcatctaaatggcagaataactcagagcatgtctttgaagatgctgggcgtctaccaccaccttatgtccccaccctacccaacaaaaataagtaaaaagaatatggtgtattctacaaatttgtggcatgctcaaagtttatgatcacataaaggcaagaggatacttcatgaataatacatttcaatgcaaataaacagatggttcacttctactagctatgagcctgtttttgtatacactgagttaatctactcaggctgtaggtcccagcaatgttctagagtctggtctttccctttcctgcagcttcgggtccttggacctttcctgtttcctattacttggagtgtctgtcagttgagcaccagttgttctggtgtttcatttgattctacttgtagcataatcatttatacgagctattgggaggttccaaaccctacctagatttgtgtaggtgatgtatcaaatgagcaatataccgttcatctgaaaatagtagcacacagccatatataggatatcattttctaaggactgtttcttcacattgagcagagcaggcataaatggtggttatttagtctaagtcttttatttttttatacctgattttcaacataacacgcaatgtggatgtcgagtagtgttaagaatggtgctgctcctgacaagtgtatgttaactgtttacattttctatctgtagaattatttctctattactgaacttttcctaagtaaaatgtctttgaagtctcgttatttctgaaatacgttgtctgtaatagacccaggcaccttttaaattatctctggaacaagagggatttcatgtaatgaactaggaaatgcatactcacataagcaacaaggttctaggcagaaagccccttggaatttgtgaccaacaggagcaagaacaggtgcggctcaacatgcaatgtctgaaaatttgcttggcattttattcatatatttagtgcaaaattatttttgagtgagatattttacatcactgttaatgtgcaatatttaagattaaaatacattagcttttttatatactttgaagtagcaagtttgttttcgatggcttagagtcatgatttccagcttcccagcctttttatcagtcccttttctaatacaacaaggtgcattaatttgattaggcaaattagagttctaagacacttcttgaattgtagacagaaaatattggattcacaatttcagcagaaatttgagaatgagtgtgtttatattaatttcacaattagctgtattttctgtagcatagattatgtcactgttgcactttcacagcagacatgctttcagaaggttctcatattttatgtttgattgctgataagccatctctattgatacagattttggttaagtaaggaaaaccaggtgtgtgtctgtatcatttattgtaaatgccagctgccacttgccaaccatcatgttcagttcaattcaaagaaaacaaactctcattacttagtgtaaactaaaatacttaacaaattatatcctaaaaacaaggtctctttgttaaatgttgcatgccctaggttttaaattactacatccaaatacagttttcgtcttaaatttgttaagctaaatatatgttggttctttttattttggaatcctttaagcatcttaaacattttttttttgaagagaagttacaaataacatttctatcaggtagtacttgtatgaaaccacctttcttattctataattttgatttttcaattttatatacttaatatactcactgtcttactatcagaaagttattttgaccaagatttttattatcttcatagattcagaaagagatgctaattctgtaccaatgtcttcctggttactattctcttccctctaatatatactggccatttgtaaaaccattgtgttgttgggatcacttagttatactatacgcagatagagcatctcaactctgtcatagtgtttgctgaacagttttcagtgtcatgcacctttggctgctaattgttcctgacgtgcactcttccgagttggtaaaggcacagtgtgttcatgccagacttctaagagaaacaccagcctcttaaatcagaagcctacacacaacccccttaacaatccaaagaagcttgatggtgtgcaaagaagcatcctgccagccttgtcattgttctgttctatgctaatcctgctgtgttgtctaaaagatggagggaagaggacatcagtgtctgatagtgaaatcatcagcaggaaagtgaagctctttccttggttacagataagacttggtttacactattggccagtatctgctaaacatatgaagacttaactattcagtgttgcctaggcattcgcctgcacaacattttgaggttagaacatagaatattttcagaaatactgttgtagtttgtgagtgttgttcattagttacacattagctatagagtggatgcatgaagccccatgacaccagtaaacttctcttaccagtaggtaaaccaaacaccattctgtcattagcagccctcttaaatgttgcctctccgtatcctgttgcatttttgtgtgcattgtgtttctactgatctctcttaggtttttacggaatcaaaggaaactaatttttccttaatagcaagaaagatgaagaggtaaagggcattgaagcagaaatgtatagtttggggtacgattagaaaactcgtaaggaaaacagaagtcctaatttcaaactgactgctcttcgttaagtgctcttaaggagagtctagtaacagtaacactttctggccatttctagtttagattctcttcgttactgaaacttttgagaaatattacctgtggattaattttgcacaatgttctattctcataatgacttacaaattaaactaggtttttattgaactacctcacactaattttctatgctttcccaagtaagctgttgccctgttagatctttactgagtgaattataaatgtgtgttaaatactttctagccaatgttgacacaataccagtaagtatgtaaagtatataccttacatcagtaagagacacgtgtaaaatctttgactgtatgtcttgcaaaattgtgctcgttgacattattactgtttttgtaagtagaaacctgctcgtgatatcggtccatttacattttacaaaaggagtaaatcttagtaaaaattttacgaagaaataaattacttttgtaggcccaatatttggtatatttttgagaagctgttaatcttttagctgaataatgaagttagactgaattacgtgtctccctggactgtgacatctattttctcattacagtttatcctggtcagcagggtgtcacacctggaaacctgagtatgatagctgacatttgcttttctccctctgcgatgtcattcctcctccattcctctccttccctgtgttccgttccctctcctttcctctagacaaaacaaaatggggcactttttagggaatgctgagatcattattgtggtttttcatcattcatgccctagtcattaaacatgcaccactggaatgtaaacaatgttatctagtatgtcaattggttataatattttaaataaaaaagaaaaaagtggtatgaaaattatgaaaaaaaaaa

SEQ ID NO: 20 - protein, 308 aa, Homo sapiens
  MISC_FEATURE: MAPK8 Isoform 5
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 21 - DNA, 6115 bp, Homo sapiens
  misc_feature: MAPK8 Isoform Alpha1
gacgtgcgcgggcgtgcgcggtgacggcccgcgtctctgttactcagccgagcggccgaggccggacgacgcggcttggattgcggagccgcgagcagcgctgggtaacggccgcggcgaccaccccggacggcccctgtccccgctggcgggcttccctgtcgccgttcgctgcgctgccggtcttgcagccttacagtcatttgtaatggttacctcagattcccctgctgtggttaacgttaaacctaggaggtcagatgctctaagtgaacatgtttagtgctgtgcctgacaaaagcaagcaatcaaaatgggacttccttgagggcaggcctgtcttgcatctgtttctccaccacctagcctggaatataactggccttatgggtttctagaaggaaaatcacagaggtaaagcatcatttcatcacatcctgtcaaggcttcttggtgaatttttggatgaagccattaaattaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgtggatttatggtctgtggggtgcattatgggagaaatggtttgccacaaaatcctctttccaggaagggactatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttagcacaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgtagaattcattttgtagtaaagtagtttattttttttaatttcaagtgatgtaatttaaaacctaagttgtgtttcaaaacagcaacaaaactgtattgtattttttttgctgtaattaactgtataatgtaaacctaattattttatcatggtttaaattttttgcatatttgctttatcttatgctgctgatttttttaactgaatttgtaagattttgtttatcaaagcaactattatgtggtgacttgcctatatcatgaattatttaagatttttatagttttttttaattagaatttatttcagatgttttgttcatgatactatccttcagggttatgtgcttatcaatgaaataaccccagaggagtgagggaaaataacttgtagccagttatattcaggaataactactgtaaatgatgaacgtgttaggagacctccaatatttgctacttgccaatcctaatttagttacaagaattggtaggcaatcctacttaattttggcaaaagccccgtcatctaaatggcagaataactcagagcatgtctttgaagatgctgggcgtctaccaccaccttatgtccccaccctacccaacaaaaataagtaaaaagaatatggtgtattctacaaatttgtggcatgctcaaagtttatgatcacataaaggcaagaggatacttcatgaataatacatttcaatgcaaataaacagatggttcacttctactagctatgagcctgtttttgtatacactgagttaatctactcaggctgtaggtcccagcaatgttctagagtctggtctttccctttcctgcagcttcgggtccttggacctttcctgtttcctattacttggagtgtctgtcagttgagcaccagttgttctggtgtttcatttgattctacttgtagcataatcatttatacgagctattgggaggttccaaaccctacctagatttgtgtaggtgatgtatcaaatgagcaatataccgttcatctgaaaatagtagcacacagccatatataggatatcattttctaaggactgtttcttcacattgagcagagcaggcataaatggtggttatttagtctaagtcttttatttttttatacctgattttcaacataacacgcaatgtggatgtcgagtagtgttaagaatggtgctgctcctgacaagtgtatgttaactgtttacattttctatctgtagaattatttctctattactgaacttttcctaagtaaaatgtctttgaagtctcgttatttctgaaatacgttgtctgtaatagacccaggcaccttttaaattatctctggaacaagagggatttcatgtaatgaactaggaaatgcatactcacataagcaacaaggttctaggcagaaagccccttggaatttgtgaccaacaggagcaagaacaggtgcggctcaacatgcaatgtctgaaaatttgcttggcattttattcatatatttagtgcaaaattatttttgagtgagatattttacatcactgttaatgtgcaatatttaagattaaaatacattagcttttttatatactttgaagtagcaagtttgttttcgatggcttagagtcatgatttccagcttcccagcctttttatcagtcccttttctaatacaacaaggtgcattaatttgattaggcaaattagagttctaagacacttcttgaattgtagacagaaaatattggattcacaatttcagcagaaatttgagaatgagtgtgtttatattaatttcacaattagctgtattttctgtagcatagattatgtcactgttgcactttcacagcagacatgctttcagaaggttctcatattttatgtttgattgctgataagccatctctattgatacagattttggttaagtaaggaaaaccaggtgtgtgtctgtatcatttattgtaaatgccagctgccacttgccaaccatcatgttcagttcaattcaaagaaaacaaactctcattacttagtgtaaactaaaatacttaacaaattatatcctaaaaacaaggtctctttgttaaatgttgcatgccctaggttttaaattactacatccaaatacagttttcgtcttaaatttgttaagctaaatatatgttggttctttttattttggaatcctttaagcatcttaaacattttttttttgaagagaagttacaaataacatttctatcaggtagtacttgtatgaaaccacctttcttattctataattttgatttttcaattttatatacttaatatactcactgtcttactatcagaaagttattttgaccaagatttttattatcttcatagattcagaaagagatgctaattctgtaccaatgtcttcctggttactattctcttccctctaatatatactggccatttgtaaaaccattgtgttgttgggatcacttagttatactatacgcagatagagcatctcaactctgtcatagtgtttgctgaacagttttcagtgtcatgcacctttggctgctaattgttcctgacgtgcactcttccgagttggtaaaggcacagtgtgttcatgccagacttctaagagaaacaccagcctcttaaatcagaagcctacacacaacccccttaacaatccaaagaagcttgatggtgtgcaaagaagcatcctgccagccttgtcattgttctgttctatgctaatcctgctgtgttgtctaaaagatggagggaagaggacatcagtgtctgatagtgaaatcatcagcaggaaagtgaagctctttccttggttacagataagacttggtttacactattggccagtatctgctaaacatatgaagacttaactattcagtgttgcctaggcattcgcctgcacaacattttgaggttagaacatagaatattttcagaaatactgttgtagtttgtgagtgttgttcattagttacacattagctatagagtggatgcatgaagccccatgacaccagtaaacttctcttaccagtaggtaaaccaaacaccattctgtcattagcagccctcttaaatgttgcctctccgtatcctgttgcatttttgtgtgcattgtgtttctactgatctctcttaggtttttacggaatcaaaggaaactaatttttccttaatagcaagaaagatgaagaggtaaagggcattgaagcagaaatgtatagtttggggtacgattagaaaactcgtaaggaaaacagaagtcctaatttcaaactgactgctcttcgttaagtgctcttaaggagagtctagtaacagtaacactttctggccatttctagtttagattctcttcgttactgaaacttttgagaaatattacctgtggattaattttgcacaatgttctattctcataatgacttacaaattaaactaggtttttattgaactacctcacactaattttctatgctttcccaagtaagctgttgccctgttagatctttactgagtgaattataaatgtgtgttaaatactttctagccaatgttgacacaataccagtaagtatgtaaagtatataccttacatcagtaagagacacgtgtaaaatctttgactgtatgtcttgcaaaattgtgctcgttgacattattactgtttttgtaagtagaaacctgctcgtgatatcggtccatttacattttacaaaaggagtaaatcttagtaaaaattttacgaagaaataaattacttttgtaggcccaatatttggtatatttttgagaagctgttaatcttttagctgaataatgaagttagactgaattacgtgtctccctggactgtgacatctattttctcattacagtttatcctggtcagcagggtgtcacacctggaaacctgagtatgatagctgacatttgcttttctccctctgcgatgtcattcctcctccattcctctccttccctgtgttccgttccctctcctttcctctagacaaaacaaaatggggcactttttagggaatgctgagatcattattgtggtttttcatcattcatgccctagtcattaaacatgcaccactggaatgtaaacaatgttatctagtatgtcaattggttataatattttaaataaaaaagaaaaaagtggtatgaaaattatgaaa

SEQ ID NO: 22 - protein, 384 aa, Homo sapiens
  MISC_FEATURE: MAPK8 Isoform Alpha1
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 23 - DNA, 5619 bp, Homo sapiens
  misc_feature: MAPK8 Isoform 6
gacgtgcgcgggcgtgcgcggtgacggcccgcgtctctgttactcagccgagcggccgaggccggacgacgcggcttggattgcggagccgcgagcagcgctgggtaacggccgcggcgaccaccccggacggcccctgtccccgctggcgggcttccctgtcgccgttcgctgcgctgccggcttcttggtgaatttttggatgaagccattaaattaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgtagaattcattttgtagtaaagtagtttattttttttaatttcaagtgatgtaatttaaaacctaagttgtgtttcaaaacagcaacaaaactgtattgtattttttttgctgtaattaactgtataatgtaaacctaattattttatcatggtttaaattttttgcatatttgctttatcttatgctgctgatttttttaactgaatttgtaagattttgtttatcaaagcaactattatgtggtgacttgcctatatcatgaattatttaagatttttatagttttttttaattagaatttatttcagatgttttgttcatgatactatccttcagggttatgtgcttatcaatgaaataaccccagaggagtgagggaaaataacttgtagccagttatattcaggaataactactgtaaatgatgaacgtgttaggagacctccaatatttgctacttgccaatcctaatttagttacaagaattggtaggcaatcctacttaattttggcaaaagccccgtcatctaaatggcagaataactcagagcatgtctttgaagatgctgggcgtctaccaccaccttatgtccccaccctacccaacaaaaataagtaaaaagaatatggtgtattctacaaatttgtggcatgctcaaagtttatgatcacataaaggcaagaggatacttcatgaataatacatttcaatgcaaataaacagatggttcacttctactagctatgagcctgtttttgtatacactgagttaatctactcaggctgtaggtcccagcaatgttctagagtctggtctttccctttcctgcagcttcgggtccttggacctttcctgtttcctattacttggagtgtctgtcagttgagcaccagttgttctggtgtttcatttgattctacttgtagcataatcatttatacgagctattgggaggttccaaaccctacctagatttgtgtaggtgatgtatcaaatgagcaatataccgttcatctgaaaatagtagcacacagccatatataggatatcattttctaaggactgtttcttcacattgagcagagcaggcataaatggtggttatttagtctaagtcttttatttttttatacctgattttcaacataacacgcaatgtggatgtcgagtagtgttaagaatggtgctgctcctgacaagtgtatgttaactgtttacattttctatctgtagaattatttctctattactgaacttttcctaagtaaaatgtctttgaagtctcgttatttctgaaatacgttgtctgtaatagacccaggcaccttttaaattatctctggaacaagagggatttcatgtaatgaactaggaaatgcatactcacataagcaacaaggttctaggcagaaagccccttggaatttgtgaccaacaggagcaagaacaggtgcggctcaacatgcaatgtctgaaaatttgcttggcattttattcatatatttagtgcaaaattatttttgagtgagatattttacatcactgttaatgtgcaatatttaagattaaaatacattagcttttttatatactttgaagtagcaagtttgttttcgatggcttagagtcatgatttccagcttcccagcctttttatcagtcccttttctaatacaacaaggtgcattaatttgattaggcaaattagagttctaagacacttcttgaattgtagacagaaaatattggattcacaatttcagcagaaatttgagaatgagtgtgtttatattaatttcacaattagctgtattttctgtagcatagattatgtcactgttgcactttcacagcagacatgctttcagaaggttctcatattttatgtttgattgctgataagccatctctattgatacagattttggttaagtaaggaaaaccaggtgtgtgtctgtatcatttattgtaaatgccagctgccacttgccaaccatcatgttcagttcaattcaaagaaaacaaactctcattacttagtgtaaactaaaatacttaacaaattatatcctaaaaacaaggtctctttgttaaatgttgcatgccctaggttttaaattactacatccaaatacagttttcgtcttaaatttgttaagctaaatatatgttggttctttttattttggaatcctttaagcatcttaaacattttttttttgaagagaagttacaaataacatttctatcaggtagtacttgtatgaaaccacctttcttattctataattttgatttttcaattttatatacttaatatactcactgtcttactatcagaaagttattttgaccaagatttttattatcttcatagattcagaaagagatgctaattctgtaccaatgtcttcctggttactattctcttccctctaatatatactggccatttgtaaaaccattgtgttgttgggatcacttagttatactatacgcagatagagcatctcaactctgtcatagtgtttgctgaacagttttcagtgtcatgcacctttggctgctaattgttcctgacgtgcactcttccgagttggtaaaggcacagtgtgttcatgccagacttctaagagaaacaccagcctcttaaatcagaagcctacacacaacccccttaacaatccaaagaagcttgatggtgtgcaaagaagcatcctgccagccttgtcattgttctgttctatgctaatcctgctgtgttgtctaaaagatggagggaagaggacatcagtgtctgatagtgaaatcatcagcaggaaagtgaagctctttccttggttacagataagacttggtttacactattggccagtatctgctaaacatatgaagacttaactattcagtgttgcctaggcattcgcctgcacaacattttgaggttagaacatagaatattttcagaaatactgttgtagtttgtgagtgttgttcattagttacacattagctatagagtggatgcatgaagccccatgacaccagtaaacttctcttaccagtaggtaaaccaaacaccattctgtcattagcagccctcttaaatgttgcctctccgtatcctgttgcatttttgtgtgcattgtgtttctactgatctctcttaggtttttacggaatcaaaggaaactaatttttccttaatagcaagaaagatgaagaggtaaagggcattgaagcagaaatgtatagtttggggtacgattagaaaactcgtaaggaaaacagaagtcctaatttcaaactgactgctcttcgttaagtgctcttaaggagagtctagtaacagtaacactttctggccatttctagtttagattctcttcgttactgaaacttttgagaaatattacctgtggattaattttgcacaatgttctattctcataatgacttacaaattaaactaggtttttattgaactacctcacactaattttctatgctttcccaagtaagctgttgccctgttagatctttactgagtgaattataaatgtgtgttaaatactttctagccaatgttgacacaataccagtaagtatgtaaagtatataccttacatcagtaagagacacgtgtaaaatctttgactgtatgtcttgcaaaattgtgctcgttgacattattactgtttttgtaagtagaaacctgctcgtgatatcggtccatttacattttacaaaaggagtaaatcttagtaaaaattttacgaagaaataaattacttttgtaggcccaatatttggtatatttttgagaagctgttaatcttttagctgaataatgaagttagactgaattacgtgtctccctggactgtgacatctattttctcattacagtttatcctggtcagcagggtgtcacacctggaaacctgagtatgatagctgacatttgcttttctccctctgcgatgtcattcctcctccattcctctccttccctgtgttccgttccctctcctttcctctagacaaaacaaaatggggcactttttagggaatgctgagatcattattgtggtttttcatcattcatgccctagtcattaaacatgcaccactggaatgtaaacaatgttatctagtatgtcaattggttataatattttaaataaaaaagaaaaaagtggtatgaaaattatgaaa

SEQ ID NO: 24 - protein, 351 aa, Homo sapiens
  MISC_FEATURE: MAPK8 Isoform 6
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENADSEHNKLKASQARDLLSKMLVIDASKRISVDEALQHPYINVWYDPSEAEAPPPKIPDKQLDEREHTIEEWKELIYKEVMDLEERTKNGVIRGQPSPLGAAVINGSQHPSSSSSVNDVSSMSTDPTLASDTDSSLEAAAGPLGCCR

SEQ ID NO: 25 - DNA, 5852 bp, Homo sapiens
  misc_feature: MAPK8 Isoform  Beta1
gacgtgcgcgggcgtgcgcggtgacggcccgcgtctctgttactcagccgagcggccgaggccggacgacgcggcttggattgcggagccgcgagcagcgctgggtaacggccgcggcgaccaccccggacggcccctgtccccgctggcgggcttccctgtcgccgttcgctgcgctgccggcttcttggtgaatttttggatgaagccattaaattaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgttgacatttggtcagttgggtgcatcatgggagaaatgatcaaaggtggtgttttgttcccaggtacagatcatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttagcacaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgtagaattcattttgtagtaaagtagtttattttttttaatttcaagtgatgtaatttaaaacctaagttgtgtttcaaaacagcaacaaaactgtattgtattttttttgctgtaattaactgtataatgtaaacctaattattttatcatggtttaaattttttgcatatttgctttatcttatgctgctgatttttttaactgaatttgtaagattttgtttatcaaagcaactattatgtggtgacttgcctatatcatgaattatttaagatttttatagttttttttaattagaatttatttcagatgttttgttcatgatactatccttcagggttatgtgcttatcaatgaaataaccccagaggagtgagggaaaataacttgtagccagttatattcaggaataactactgtaaatgatgaacgtgttaggagacctccaatatttgctacttgccaatcctaatttagttacaagaattggtaggcaatcctacttaattttggcaaaagccccgtcatctaaatggcagaataactcagagcatgtctttgaagatgctgggcgtctaccaccaccttatgtccccaccctacccaacaaaaataagtaaaaagaatatggtgtattctacaaatttgtggcatgctcaaagtttatgatcacataaaggcaagaggatacttcatgaataatacatttcaatgcaaataaacagatggttcacttctactagctatgagcctgtttttgtatacactgagttaatctactcaggctgtaggtcccagcaatgttctagagtctggtctttccctttcctgcagcttcgggtccttggacctttcctgtttcctattacttggagtgtctgtcagttgagcaccagttgttctggtgtttcatttgattctacttgtagcataatcatttatacgagctattgggaggttccaaaccctacctagatttgtgtaggtgatgtatcaaatgagcaatataccgttcatctgaaaatagtagcacacagccatatataggatatcattttctaaggactgtttcttcacattgagcagagcaggcataaatggtggttatttagtctaagtcttttatttttttatacctgattttcaacataacacgcaatgtggatgtcgagtagtgttaagaatggtgctgctcctgacaagtgtatgttaactgtttacattttctatctgtagaattatttctctattactgaacttttcctaagtaaaatgtctttgaagtctcgttatttctgaaatacgttgtctgtaatagacccaggcaccttttaaattatctctggaacaagagggatttcatgtaatgaactaggaaatgcatactcacataagcaacaaggttctaggcagaaagccccttggaatttgtgaccaacaggagcaagaacaggtgcggctcaacatgcaatgtctgaaaatttgcttggcattttattcatatatttagtgcaaaattatttttgagtgagatattttacatcactgttaatgtgcaatatttaagattaaaatacattagcttttttatatactttgaagtagcaagtttgttttcgatggcttagagtcatgatttccagcttcccagcctttttatcagtcccttttctaatacaacaaggtgcattaatttgattaggcaaattagagttctaagacacttcttgaattgtagacagaaaatattggattcacaatttcagcagaaatttgagaatgagtgtgtttatattaatttcacaattagctgtattttctgtagcatagattatgtcactgttgcactttcacagcagacatgctttcagaaggttctcatattttatgtttgattgctgataagccatctctattgatacagattttggttaagtaaggaaaaccaggtgtgtgtctgtatcatttattgtaaatgccagctgccacttgccaaccatcatgttcagttcaattcaaagaaaacaaactctcattacttagtgtaaactaaaatacttaacaaattatatcctaaaaacaaggtctctttgttaaatgttgcatgccctaggttttaaattactacatccaaatacagttttcgtcttaaatttgttaagctaaatatatgttggttctttttattttggaatcctttaagcatcttaaacattttttttttgaagagaagttacaaataacatttctatcaggtagtacttgtatgaaaccacctttcttattctataattttgatttttcaattttatatacttaatatactcactgtcttactatcagaaagttattttgaccaagatttttattatcttcatagattcagaaagagatgctaattctgtaccaatgtcttcctggttactattctcttccctctaatatatactggccatttgtaaaaccattgtgttgttgggatcacttagttatactatacgcagatagagcatctcaactctgtcatagtgtttgctgaacagttttcagtgtcatgcacctttggctgctaattgttcctgacgtgcactcttccgagttggtaaaggcacagtgtgttcatgccagacttctaagagaaacaccagcctcttaaatcagaagcctacacacaacccccttaacaatccaaagaagcttgatggtgtgcaaagaagcatcctgccagccttgtcattgttctgttctatgctaatcctgctgtgttgtctaaaagatggagggaagaggacatcagtgtctgatagtgaaatcatcagcaggaaagtgaagctctttccttggttacagataagacttggtttacactattggccagtatctgctaaacatatgaagacttaactattcagtgttgcctaggcattcgcctgcacaacattttgaggttagaacatagaatattttcagaaatactgttgtagtttgtgagtgttgttcattagttacacattagctatagagtggatgcatgaagccccatgacaccagtaaacttctcttaccagtaggtaaaccaaacaccattctgtcattagcagccctcttaaatgttgcctctccgtatcctgttgcatttttgtgtgcattgtgtttctactgatctctcttaggtttttacggaatcaaaggaaactaatttttccttaatagcaagaaagatgaagaggtaaagggcattgaagcagaaatgtatagtttggggtacgattagaaaactcgtaaggaaaacagaagtcctaatttcaaactgactgctcttcgttaagtgctcttaaggagagtctagtaacagtaacactttctggccatttctagtttagattctcttcgttactgaaacttttgagaaatattacctgtggattaattttgcacaatgttctattctcataatgacttacaaattaaactaggtttttattgaactacctcacactaattttctatgctttcccaagtaagctgttgccctgttagatctttactgagtgaattataaatgtgtgttaaatactttctagccaatgttgacacaataccagtaagtatgtaaagtatataccttacatcagtaagagacacgtgtaaaatctttgactgtatgtcttgcaaaattgtgctcgttgacattattactgtttttgtaagtagaaacctgctcgtgatatcggtccatttacattttacaaaaggagtaaatcttagtaaaaattttacgaagaaataaattacttttgtaggcccaatatttggtatatttttgagaagctgttaatcttttagctgaataatgaagttagactgaattacgtgtctccctggactgtgacatctattttctcattacagtttatcctggtcagcagggtgtcacacctggaaacctgagtatgatagctgacatttgcttttctccctctgcgatgtcattcctcctccattcctctccttccctgtgttccgttccctctcctttcctctagacaaaacaaaatggggcactttttagggaatgctgagatcattattgtggtttttcatcattcatgccctagtcattaaacatgcaccactggaatgtaaacaatgttatctagtatgtcaattggttataatattttaaataaaaaagaaaaaagtggtatgaaaattatgaaa

SEQ ID NO: 26 - protein, 384 aa, Homo sapiens
  MISC_FEATURE: MAPK8 Isoform  Beta1
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDIWSVGCIMGEMIKGGVLFPGTDHIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 27 - DNA, 6550 bp, Homo sapiens
  misc_feature: ATP6V0A2
gggggcggaaccggaaccggcagtggcttgggggcgggacctcgcggactgctgtggcggcagctggagcggcggccgcggtggcagaaccgggggcggccgctgcagtctggagccccatagtgcggggccgcggccaggccacaggaagagctcgaggcccgggccgcaccggctgagtgtgcgggcccgcgcggctcggagccgccgccgcccatcgagcccctccgggcgcgggtcggcccgccatggggtccctgttccggagcgagaccatgtgcctggcgcagctcttcctgcagtcgggcacggcctacgagtgcctcagcgccctgggcgagaaaggcctggtccagttccgagacctcaaccagaacgtaagttctttccaaagaaaatttgttggtgaggtgaagaggtgtgaagagctagagcgaatattggtgtatttggtacaggaaattaatagagctgatattccccttcctgaaggagaggccagccctcctgcgccacccctgaaacaggttctagaaatgcaggagcagttgcagaagctcgaggttgaactgagagaagtcactaagaacaaggagaaactgaggaaaaacttgctggaactgatagagtacactcacatgctgagagtgacaaagacctttgtgaaacgcaatgttgagtttgaacccacttatgaagaattcccttccttagagagtgattctttgttggattacagctgtatgcagaggctgggagcaaaactgggatttgtgtctggcctaattaaccaaggaaaagtggaagcatttgaaaaaatgttgtggagagtctgcaaagggtacaccatcgtgtcctatgcagaactggatgaatcccttgaagaccctgaaacaggggaagtcataaaatggtatgtctttttaatatccttttggggagagcagattggccacaaggttaagaagatatgtgattgctaccactgccacgtgtacccctatccaaacacagccgaggagcggagggagatccaggaggggctgaacacccgcatccaggatctctacactgtactgcacaaaaccgaggactatttgaggcaagtgctatgtaaagccgccgagtctgtctacagccgtgtgatccaggtgaagaaaatgaaggccatctatcacatgctgaacatgtgcagctttgacgtgaccaacaagtgcctcattgctgaggtctggtgtcccgaggcggatctgcaggacctgcgccgggcactggaggagggctcgagagagagtggtgctacaatcccctcattcatgaatataatccccacaaaagaaacaccccccactcggatccgcaccaacaaattcaccgagggatttcagaacatcgtggatgcttatggagtcggaagctacagagaagtcaatccagctctctttaccatcatcaccttcccgtttttatttgctgtgatgtttggagacttcggacatggctttgtgatgtttttatttgccctcttgttggtgttaaatgaaaatcatcccagactaaatcagtcacaagagatcatgaggatgttttttaatggccggtacatcctcctgctgatggggctgttctcagtgtacactggcctcatctacaacgactgcttttcaaagtcagtcaacctgttcggctctgggtggaacgtgtcggccatgtacagctccagccacccacccgcagagcataagaagatggtgctttggaatgacagcgtcgttagacacaacagcattttgcagctggatccaagcattcctggagtgttccgaggcccttatccccttggcattgatcctatttggaacttggccacaaatcgcctcacttttctaaactctttcaaaatgaaaatgtccgtgattttaggaatcattcatatgacttttggagtcattctgggaatatttaaccacttgcacttcaggaagaagttcaacatttacctggtttccatcccggaacttctcttcatgctctgtatctttggataccttatatttatgattttctacaagtggctggttttttcagcagaaacctccagagttgctcccagcattctgattgaatttattaacatgtttttattcccagccagtaaaacaagtggcctttacacagggcaggagtatgtccagagagtgctgctggttgtcacagcattgtctgtccctgtcctcttcttgggaaagccactgtttttgttgtggcttcacaatgggcgtagttgcttcggggtgaaccggagtggctacacacttataaggaaagatagtgaggaagaagtttcattgctgggaagccaagatatagaagagggaaatcaccaggtggaagatggatgtagagaaatggcgtgtgaagagtttaattttggagaaatattaatgacccaagtaatccattccatcgagtactgtctgggatgcatctccaacaccgcctcctacctgaggctctgggcgcttagcctggctcacgcacagttgtctgatgtcctgtgggccatgctgatgcgcgtgggcctccgcgttgacaccacctatggcgtcttgctactgctcccggttatcgcgctctttgcagttttgaccattttcatccttctgatcatggaagggctttctgcgtttcttcacgccatacgcctccactgggtagaatttcagaacaaattctacgttggtgcaggcaccaaatttgttcctttctcattcagtctactttcatcaaagttcaataacgacgacagtgtggcatgatcatattgctgtaaccaacaagctttcagatttatggagaatgaccatgttatagactttcacttatgtcagatttatgataggaaaaattccatcttcattactgccttatgacatagccaaataattctgtaagatatacctcttcctcatatgttaaatattttgtaaagtttaccaatttgagatataaaaatttcttttggttttttatgatgagcaaatataagttaatgccaaacgttatgttaaagttattttttcaaatacaggatttggggagagaagccaattttgcatggctagttgaaaatggtattagatacttatttcctttaaactttatttaaaacaaacaacagagttaatctgtcacctgaagttgccagataatattttccaacagctgaagtagggatagttttttaaaaaatttaatgatgggtaatcaaaacattcacatttattctgctggatttaaaaatatgtactttttttttttttttgagatgaagtcttgctgtgtcacgtaggctggagtgcaatggcacgatcgtgtctcattgcaacttccgcctcctggattcaggtgattctcctgtctcagcctcccgagtaactgggattacaggcatgcgccaccgtgaccagctaatttttttgtatttttgtagagatggggtttcaccatgttggccaggctggtctcgaacttctgacctcaagtgatccacctgcctcggcctccctaagtgctgggattacaggcgtgagccaccatgcctggccaaaatatgcaaatattttttatcctgtgctacctagaattacaagtagaggattgtttttacaattttgtcttgttttagtaattggggacaatgggataaaatgacatacaacaaatggttatcccacttgtatatttttttaaagactcttatcctgttacccttacataaaaccagttatgataattgtatgtttggaagataaattgtatgctgagccttaaaacccagtgtgactgtagaagatataatgcggacatgatttgaatttagtgacagcttggctgatgtcactgaaccagaagggtttatactgagtgaaggaaaggtaaaaagtagtattttgtatatttttgtaacaaaatataaatgaagatattttaccatgaagagattgcacttatccttgagaacagtctaaatataaatgaagatattttaccatgaagagattgcacttatccttgagaacagtctaatgatactttttttactttcagactgtttctagctaaagttctgataaaagtattgagttaatttaaactttcaaacactgtttagatcagttgaaaactaattcttaatagccattgttttataatctttagtttaaattcatctgtttgacatttggttttttgtgaacttgagtcagtcctattgaacattttgacctatattcttgcaaattatgttaatttttcatattttagtttgattttagacaattattttcaaaaagaaacctgactttttttcaccttcttgacattgtgagtgatacacgttaaaaggctatttgcccgcatcctcctctatcctgtttcgaggtagagagcttggcacttctgttacaagctgagagtctggaggtgctgagtttaacgtggctgtgcagctgatttcaaggcatgagctgaacagcgttgacacagccgtgacttacaattaaaccctttcaattacagtacagattctgtgtgctacttgatgagatgttacccaaggccagtttggacttactgctttttgtccctcatattaaattatcaaatgatgccgatgaaatgcttgaaaatgattctgctctcaaagttatgcttccctagaaaggtgtcatgtcgggcacttgtgttgggtgactatgacatcccagcagtgctggtcaggtctcagagttgctttcacctagagctgatgcttccacaaggggacatttgtgttactttgctccaggggtctgtcataaaaacaatggtaactacttcatccctaggaagcccaagacttcagggcactgttagctgattggtaaataagggacacgatattcacgggaattgtttactgctccccactagtaattctcatggggccacttttttaactctttctaattaacacctcttctcagcatagagcagaggatgcagtcatttctctgttgaaatctatgtagtatttatgtagaatgtcattatatgaaaagcgaattcagaatcctaactctgggaaagcgctctctggaaatgtagtttgataatagtgtcttataggggccacgggaatttgtttctctataaagcgatgggctccagtgtcatgttaccagagttatcgctcagttttgtttgttaaggcttttgctagaacctattatgtatccagtttttaaaacatacttcattcccttgatagcagagatccagagcacagatggcaagttgtgtggatgatactgaatatgtttattttcctaaagataaatgtcacgacacgacatttctcagtttttatttagatgtttattaccatgtgttaacagaatcttaaaacccaagggatttcttcagtaaactagactttgattcaacctgctgggctggagactgtaattgccgtggtcagttaaggagtgcaaagggtgttctgtctgataggatctgggtgagccctgcggactttccagcagcgaaaggacttggaactcctctttcgctgaatgctgctctgtttctggaatggatattttataacaacattctggtgtcagcatcccctcagcagttgtttatttaaaaatttttttattttttttgagacagtgtctcactctgtcacctaggctgaagtgcagtggtgcagtctcggctcactgcaacctctgcctcctgggtttaagcaattctcctgccctagcctcccgagtagctgggattacaggcgcctgccacagtgcccggctaatttttgtatttttagtagagacagggtttcaccatgttggccaggctggtctcaaactcctgacctcaggtgatctacccaccttggcctcccaaagtgctgggattacaggcgtgagccaccgcgcctggccctctgcagttgtttaataaggcacagaatacctgtagcataggtcagccttacgatgtccatgaattacatattcagacgttttagagcctgatacattttggaaaagaaaaacaacttctacacctattctacagtccgcatttaaaacaataaattcctctattaaaaacgtaaagccgggtttgcttgcgtgccacagggaatatatccaggaaggttattatgaagctgtcaaatcaagatgatgggaataaggcagtttgaacgaacagtcttcccacagtcaggccatttttgctgatttggtttagaattttcagaaatacttagtacactccacctgttctttgatgggaatatctaagaaggctaggtaggttcttagggttagcctgagtcatctaggggctcaactccttgtgaggggaaatgacagtgaacaagttagtactttgctccacaaatgcatgaaaggacaaatttgcatcttctatcagtatttaacttccttttgctaatgacaaataaatatatctatatttttaaaaaaaaaaa

SEQ ID NO: 28 - protein, 856 aa, Homo sapiens
  MISC_FEATURE: ATP6V0A2
MGSLFRSETMCLAQLFLQSGTAYECLSALGEKGLVQFRDLNQNVSSFQRKFVGEVKRCEELERILVYLVQEINRADIPLPEGEASPPAPPLKQVLEMQEQLQKLEVELREVTKNKEKLRKNLLELIEYTHMLRVTKTFVKRNVEFEPTYEEFPSLESDSLLDYSCMQRLGAKLGFVSGLINQGKVEAFEKMLWRVCKGYTIVSYAELDESLEDPETGEVIKWYVFLISFWGEQIGHKVKKICDCYHCHVYPYPNTAEERREIQEGLNTRIQDLYTVLHKTEDYLRQVLCKAAESVYSRVIQVKKMKAIYHMLNMCSFDVTNKCLIAEVWCPEADLQDLRRALEEGSRESGATIPSFMNIIPTKETPPTRIRTNKFTEGFQNIVDAYGVGSYREVNPALFTIITFPFLFAVMFGDFGHGFVMFLFALLLVLNENHPRLNQSQEIMRMFFNGRYILLLMGLFSVYTGLIYNDCFSKSVNLFGSGWNVSAMYSSSHPPAEHKKMVLWNDSVVRHNSILQLDPSIPGVFRGPYPLGIDPIWNLATNRLTFLNSFKMKMSVILGIIHMTFGVILGIFNHLHFRKKFNIYLVSIPELLFMLCIFGYLIFMIFYKWLVFSAETSRVAPSILIEFINMFLFPASKTSGLYTGQEYVQRVLLVVTALSVPVLFLGKPLFLLWLHNGRSCFGVNRSGYTLIRKDSEEEVSLLGSQDIEEGNHQVEDGCREMACEEFNFGEILMTQVIHSIEYCLGCISNTASYLRLWALSLAHAQLSDVLWAMLMRVGLRVDTTYGVLLLLPVIALFAVLTIFILLIMEGLSAFLHAIRLHWVEFQNKFYVGAGTKFVPFSFSLLSSKFNNDDSVA

SEQ ID NO: 29 - DNA, 3936 bp, Homo sapiens
  misc_feature: FGF7
agttttaattgcttccaatgaggtcagcaaaggtatttatcgaaaagccctgaataaaaggctcacacacacacacaagcacacacgcgctcacacacagagagaaaatccttctgcctgttgatttatggaaacaattatgattctgctggagaacttttcagctgagaaatagtttgtagctacagtagaaaggctcaagttgcaccaggcagacaacagacatggaattcttatatatccagctgttagcaacaaaacaaaagtcaaatagcaaacagcgtcacagcaactgaacttactacgaactgtttttatgaggatttatcaacagagttatttaaggaggaatcctgtgttgttatcaggaactaaaaggataaggctaacaatttggaaagagcaactactctttcttaaatcaatctacaattcacagataggaagaggtcaatgacctaggagtaacaatcaactcaagattcattttcattatgttattcatgaacacccggagcactacactataatgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaattgcatatggtatataaagaaccagttccagcagggagatttctttaagtggactgttttctttcttctcaaaattttctttccttttattttttagtaatcaagaaaggctggaaaactactgaaaaactgatcaagctggacttgtgcatttatgtttgttttaagacactgcattaaagaaagatttgaaaagtatacacaaaaatcagatttagtaactaaaggttgtaaaaaattgtaaaactggttgtacaatcatgatgttagtaacagtaatttttttcttaaattaatttacccttaagagtatgttagatttgattatctgataatgattatttaaatattcctatctgcttataaaatggctgctataataataataatacagatgttgttatataaggtatatcagacctacaggcttctggcaggatttgtcagataatcaagccacactaactatggaaaatgagcagcattttaaatgctttctagtgaaaaattataatctacttaaactctaatcagaaaaaaaattctcaaaaaaactattatgaaagtcaataaaatagataatttaacaaaagtacaggattagaacatgcttatacctataaataagaacaaaatttctaatgctgctcaagtggaaagggtattgctaaaaggatgtttccaaaaatcttgtatataagatagcaacagtgattgatgataatactgtacttcatcttacttgccacaaaataacattttataaatcctcaaagtaaaattgagaaatctttaagtttttttcaagtaacataatctatctttgtataattcatatttgggaatatggcttttaataatgttcttcccacaaataatcatgcttttttcctatggttacagcattaaactctattttaagttgtttttgaactttattgttttgttatttaagtttatgttatttataaaaaaaaaaccttaataagctgtatctgtttcatatgcttttaattttaaaggaataacaaaactgtctggctcaacggcaagtttccctcccttttctgactgacactaagtctagcacacagcacttgggccagcaaatcctggaaggcagacaaaaataagagcctgaagcaatgcttacaatagatgtctcacacagaacaatacaaatatgtaaaaaatctttcaccacatattcttgccaattaattggatcatataagtaaaatcattacaaatataagtatttacaggattttaaagttagaatatatttgaatgcatgggtagaaaatatcatattttaaaactatgtatatttaaatttagtaattttctaatctctagaaatctctgctgttcaaaaggtggcagcactgaaagttgttttcctgttagatggcaagagcacaatgcccaaaatagaagatgcagttaagaataaggggccctgaatgtcatgaaggcttgaggtcagcctacagataacaggattattacaaggatgaatttccacttcaaaagtctttcattggcagatcttggtagcactttatatgttcaccaatgggaggtcaatatttatctaatttaaaaggtatgctaaccactgtggttttaatttcaaaatatttgtcattcaagtccctttacataaatagtatttggtaatacatttatagatgagagttatatgaaaaggctaggtcaacaaaaacaatagattcatttaattttcctgtggttgacctatacgaccaggatgtagaaaactagaaagaactgcccttcctcagatatactcttgggagagagcatgaatggtattctgaactatcacctgattcaaggactttgctagctaggttttgaggtcaggcttcagtaactgtagtcttgtgagcatattgagggcagaggaggacttagtttttcatatgtgtttccttagtgcctagcagactatctgttcataatcagttttcagtgtgaattcactgaatgtttatagacaaaagaaaatacacactaaaactaatcttcattttaaaagggtaaaacatgactatacagaaatttaaatagaaatagtgtatatacatataaaatacaagctatgttaggaccaaatgctctttgtctatggagttatacttccatcaaattacatagcaatgctgaattaggcaaaaccaacatttagtggtaaatccattcctggtagtataagtcacctaaaaaagacttctagaaatatgtactttaattatttgtttttctcctatttttaaatttattatgcaaattttagaaaataaaatttgctctagttacacacctttagaattctagaatattaaaactgtaaggggcctccatccctcttactcatttgtagtctaggaaattgagattttgatacacctaaggtcacgcagctgggtagatatacagctgtcacaagagtctagatcagttagcacatgctttctactcttcgattattagtattattagctaatggtctttggcatgtttttgttttttatttctgttgagatatagcctttacatttgtacacaaatgtgactatgtcttggcaatgcacttcatacacaatgactaatctatactgtgatgatttgactcaaaaggagaaaagaaattatgtagttttcaattctgattcctattcaccttttgtttatgaatggaaagctttgtgcaaaatatacatataagcagagtaagccttttaaaaatgttctttgaaagataaaattaaatacatgagtttctaacaattaga

SEQ ID NO: 30 - protein, 194 aa, Homo sapiens
  MISC_FEATURE: FGF7
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT